(19) United States
(12) Patent Application Publication
(10) Pub. No.: US 2023/0025280 A1
(43) Pub. Date: Jan. 26, 2023

(54) PH RESPONSIVE COMPOSITIONS, FORMULATIONS, AND METHODS OF IMAGING A TUMOR

(71) Applicant: ONCONANO MEDICINE, INC, Southlake, TX (US)

(72) Inventors: Tian ZHAO, Southlake, TX (US); Yalia JAYALAKSHMI, Southlake, TX (US); Keith A. HALL, Southlake, TX (US); Brian MADAJEWSKI, Southlake, TX (US); Hargita KAPLAN, Southlake, TX (US)

(21) Appl. No.: 17/756,190

(22) PCT Filed: Nov. 17, 2020
(86) PCT No.: PCT/US20/60910

§ 371 Date: May 18, 2022

Related U.S. Application Data
(60) Provisional application No. 62/937,141, filed on Nov. 18, 2019.

(51) Int. Cl. A61K 49/00; G01N 33/84; G01N 33/58

(52) U.S. Cl. CPC A61K 49/0054; G01N 33/84; A61K 49/0082; G01N 33/582; G01N 2800/7028

(57) ABSTRACT

Described herein are formulations, methods, and pH responsive compositions useful for the detection of primary and metastatic tumor tissues.

### CROSS REFERENCE

This application claims the benefit of U.S. Provisional Application No. 62/937,141, filed Nov. 18, 2019, which is hereby incorporated by reference in its entirety.

### STATEMENT AS TO FEDERALLY SPONSORED RESEARCH

This invention was made with government support under CA217528 awarded by the National Institutes of Health. The government has certain rights in the invention.

### BACKGROUND OF THE DISCLOSURE

Approximately 1.7 million new cancer cases are expected to be diagnosed and approximately 610,000 Americans are expected to die of cancer in 2019. Effective imaging agents are needed for the detection of primary and metastatic tumor tissue.

Treatment guidelines for solid cancers of all stages prominently include surgical removal of the primary tumor, as well as at risk or involved lymph nodes. Despite the biological and anatomical differences between these tumor types, the post-operative margin status is one of the most important prognostic factors of local tumor control and therefore the chance for recurrent disease or tumor metastasis.

Surgical excision of solid tumors is a balance between oncologic efficacy and minimization of the resection of normal tissue, and thus functional morbidity as well as cosmesis. This also holds true for lymphadenectomy performed for diagnostic and therapeutic purposes, often at the same time as the removal of the primary cancer. The presence or absence of lymph node metastasis is the most important determinant of survival for gastrointestinal cancers, breast cancers, and many other solid cancers. While physical examination or imaging modalities used for staging are successful in detecting enlarged or abnormal nodes and help with surgical treatment plans, for a high percentage of patients, lymph node metastasis is present at a level that is too small to be detected by current methods, which leads to under-staging. Because occult nodal metastasis is common, elective regional nodal dissection and histological examination is standard of care for many solid cancers, especially when locally advanced. This leads to overtreatment with significant potential for treatment related morbidities.

Optical imaging strategies have rapidly been adapted to image tissues intra-operatively based on cellular imaging, native auto fluorescence and Raman scattering. Optical imaging offers the potential for real-time feedback during surgery and there are a variety of readily available camera systems that provide a wide view of the surgical field. One strategy to overcome the complexity encountered due to the diversity in oncogenotypes and histologic phenotypes during surgery is to target metabolic vulnerabilities that are ubiquitous in cancer. Aerobic glycolysis, known as the Warburg effect, in which cancer cells preferentially uptake glucose and convert it to lactic acid, occurs in all solid cancers and represents one such target.

### SUMMARY OF THE DISCLOSURE

In some cases, compositions presented herein exploit pH as a universal biomarker for solid cancers where the ubiquitous pH difference between cancerous tissue and normal tissue and provides a highly sensitive and specific fluorescence response after being taken up by the cells, thus, allowing the detection of tumor tissue, tumor margin, and metastatic tumors including lymph nodes and peritoneal metastasis.

In some cases, compounds described herein are imaging agents useful for the detection of primary and metastatic tumor tissue (including lymph nodes). Real-time fluorescence imaging during surgery aids surgeon in the delineation of tumor tissue versus normal tissue, with the goal of achieving negative margins and complete tumor resection, as well as in the detection of metastatic lymph nodes. Clinical benefits from the improved surgical outcomes include such as reduced tumor recurrence and re-operation rates, avoidance of unnecessary surgeries, preservation of function, comesis, and informing patient treatment plans.

In certain embodiments, provided herein is a block copolymer of Formula (II), or a pharmaceutically acceptable salt, solvate, or hydrate thereof:

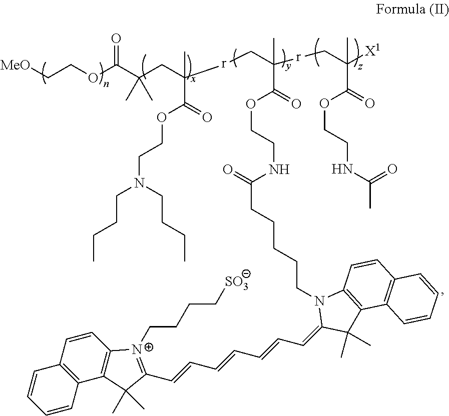

wherein: n=90-140; x is 50-200; y is 0-3; z is 0-3; and X¹ is a halogen, —OH, or —C(O)OH.

In some embodiments, X¹ is a halogen. In some embodiments, X¹ is —Br. In some embodiments, n is 100-120. In some embodiments, n is 113. In some embodiments, x is 60-150. In some embodiments, y is 0.5-1.5. In some embodiments, y is 0. In some embodiments, z is 1.5-2.5. In some embodiments, z is 0.

In certain embodiments, provided herein is a micelle comprising one or more block copolymers of Formula (II), or a pharmaceutically acceptable salt, solvate, or hydrate thereof.

In certain embodiments, provided herein is a pH responsive composition comprising a pH transition point and an emission spectrum. In some embodiments, the pH transition point is between 4.8-5.5. In some embodiments, the pH transition point is about 4.8, 4.9, 5.0, 5.1, 5.2, 5.3, 5.4, or 5.5. In some embodiments, the emission spectrum is between 700-900 nm. In some embodiments, the composition has a pH transition range (ΔpH_{10-90%}) of less than 1 pH unit. In some embodiments, the pH transition range is less than 0.25 pH units. In some embodiments, the pH transition range is less than 0.15 pH units. In some embodiments, the composition has a fluorescence activation ratio of greater than 25. In some embodiments, the composition has a fluorescence activation ratio of greater than 50.

In certain embodiments, provided herein is an imaging agent comprising one or more block copolymers having the structure of Formula (II), or a pharmaceutically acceptable salt, solvate, or hydrate thereof. In some embodiments, the imaging agent comprises poly(ethyleneoxide)-b-poly(dibutylaminoethyl methacrylate-r-aminoethylmethylacrylate hydrochloride) copolymer indocyanine green and acetic acid conjugate.

In certain embodiments, provided herein is a pharmaceutical composition comprising a micelle, wherein the micelle comprises 1) one or more block copolymers having the structure of Formula (II), or a pharmaceutically acceptable salt, solvate or hydrate thereof:

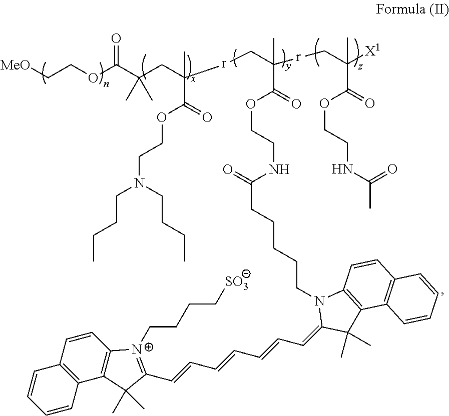

wherein: n is 90-140; x is 50-200; y is 0-3; z is 0-3; and X¹ is a halogen, —OH, or —C(O)OH; and 2) a stabilizing agent.

In some embodiments, the stabilizing agent is a cryoprotectant. In some embodiments, the stabilizing agent is a sugar, a sugar derivative, a detergent or a salt. In some embodiments, the stabilizing agent is a monosaccharide, disaccharide, trisaccharide, water soluble polysaccharide, or sugar alcohol, or combination thereof. In some embodiments, the stabilizing agent is fructose, galactose, glucose, lactose, sucrose, trehalose, maltose, mannitol, sorbitol, ribose, dextrin, cyclodextrin, maltodextrin, raffinose, or xylose, or a combination thereof. In some embodiments, the stabilizing agent is trehalose.

In some embodiments, the pharmaceutical composition comprises from about 0.5% to about 25% w/v, from about 1% to about 20% w/v, from about 5% to about 15% w/v, from about 6% to about 13% w/v, from about 7% to about 12% w/v, or from about 8% to about 11% w/v of the stabilizing agent. In certain embodiments, the pharmaceutical composition comprises about 5% w/v, about 6% w/v, about 7% w/v, about 8% w/v, about 9% w/v, about 10% w/v, about 11% w/v, about 12% w/v, about 13% w/v, about 14% w/v, or about 15% w/v of the stabilizing agent.

In some embodiments, the pharmaceutical composition further comprises a liquid or aqueous carrier. In some embodiments, the liquid carrier is selected from sterile water, saline, D5W, or ringers lactate solution.

In some embodiments, the pharmaceutical composition comprises about from 1.0 mg/mL to about 5.0 mg/mL of the block copolymer of Formula (II). In some embodiments, the pharmaceutical composition comprises about from 0.1 mg/kg to about 3 mg/kg or from about 0.1 to about 1.2 mg/kg of the block copolymer of Formula (II). In some embodiments, the pharmaceutical composition comprises about 1 mg/kg, 2 mg/kg, 3 mg/kg, about 4 mg/kg, about 5 mg/kg, about 6 mg/kg, or about 7 mg/kg of the block copolymer of Formula (II). In some embodiments, the composition comprising about 0.1 mg/kg, 0.3 mg/kg, 0.5 mg/kg, 0.8 mg/kg, 1 mg/kg, 1.2 mg/kg, 1.4 mg/kg, 1.6 mg/kg, 1.8 mg/kg, 2 mg/kg, 2.5 mg/kg, or 3 mg/kg of the block copolymer of Formula (II).

In another aspect, provided herein is a pharmaceutical composition comprising about 3 mg/mL of a block copolymer having the structure of Formula (II), or a pharmaceutically acceptable salt, solvate, or hydrate thereof:

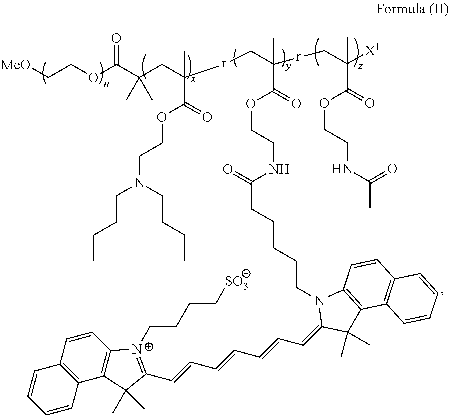

wherein: n is 90-140, x is 60-150, y is 0-3; z is 0-3; and X¹ is Br; and about 10% w/v trehalose in water. In some embodiments, the pharmaceutical composition is formulated for oral, intramuscular, subcutaneous, intratumoral, or intravenous administration. In certain embodiments, the pharmaceutical composition is formulated for intravenous (I.V.) administration.

In another aspect, provided herein is a method of imaging the pH of an intracellular or extracellular environment comprising: (a) contacting a pharmaceutical composition of the present disclosure with the environment; and (b) detecting one or more optical signals from the environment, wherein a detected optical signal indicates that the micelle has reached its pH transition point and disassociated. In some embodiments, the optical signal is a fluorescent signal. In some embodiments, the intracellular environment is imaged, the cell is contacted with the pH responsive composition under conditions suitable to cause uptake of the pH responsive composition. In some embodiments, the intracellular environment is part of a cell. In some embodiments, the extracellular environment is of a tumor or vascular cell. In some embodiments, the extracellular environment is intravascular or extravascular. In some embodiments, the tumor is solid tumor. In some embodiments, the tumor is of a cancer, wherein the cancer is of the breast, colorectal, bladder, esophageal, head and neck (HNSSC), lung, brain, prostate, ovary, or skin (including melanoma and sarcoma).

In another aspect, provided herein is a method of resecting a tumor in a patient comprising: (a) detecting one or more optical signals from the tumor or a sample thereof from the patient administered with an effective dose of a pharmaceutical composition described herein, wherein a detected optical signal(s) indicate the presence of the tumor; and (b) resecting the tumor via a surgery. In some embodiments, the optical signals indicate the margins of the tumor. In some embodiments, tumor is at least 90%, 95%, or 99% resected. In some embodiments, the cancer is breast cancer, head and neck squamous cell carcinoma (NHSCC), lung cancer, ovarian cancer, prostate cancer, bladder cancer, urethral cancer, esophageal cancer, brain cancer, pancreatic cancer, skin cancer, melanoma, sarcoma, pleural metastasis, kidney cancer, lymph node cancer, cervical cancer, or colorectal cancer. In some embodiments, the cancer is breast cancer, head and neck squamous cell carcinoma (NHSCC), esophageal cancer, colorectal cancer, ovarian cancer, or prostate cancer.

In some embodiments, the pharmaceutical composition disclosed herein is administered prior to a surgery. In some embodiments, the pharmaceutical composition is administered prior to imaging a tumor or lymph node. In some embodiments, the pharmaceutical composition disclosed herein is administered prior to patient management of clinical outcomes. In some embodiments, the pharmaceutical composition is administered at least 1 hour, at least 2 hours, at least 4 hours, at least 6 hours, at least 8 hours, at least 10 hours, at least 12 hours, at least 14 hours, at least 16 hours, at least 18 hours, at least 20 hours, at least 24 hours, at least 28 hours, at least 32 hours, at least 80 hours, at least 1 day, at least 2 days, at least 3 days, at least 4 days, at least 5 days, at least 6 days, at least 1 week, or at least 2 weeks prior to a surgery. In some embodiments, the pharmaceutical composition is administered from about 1 hour to about 32 hours, about 2 hours to about 32 hours, 16 hours to about 32 hours, about 20 hours to about 28 hours, about 1 hour to about 5 hours, or about 3 hours to about 9 hours prior to a surgery. In some embodiments, the pharmaceutical composition is administered as an injection or an infusion. In some embodiments, the pharmaceutical composition is administered as a single dose or as multiple doses.

In another aspect, provided herein is a method of treating cancer, the method comprising: (a) detecting one or more optical signals in a cancer patient in need thereof administered with an effective dose of a pharmaceutical composition described herein, wherein a detected optical signal indicates the presence of the cancerous tumor. In some embodiments, the method further comprising imaging body cavity of the cancer patient, or imaging the cancerous tumor or a slice or specimen thereof (e.g., fresh or formalin fixed), optionally by back-table fluorescence-guided imaging after the removal from the patient.

In another aspect, provided herein is a method of minimizing recurrence of cancer for at least five years, the method comprising: (a) detecting one or more optical signals in a cancer patient in need thereof administered with an effective dose of a pharmaceutical composition disclosed herein, wherein a detected optical signal indicates the presence of a cancerous tumor, and wherein the presence of the tumor indicates the recurrence of the cancer; and (b) treating the cancer to minimize the recurrence if the one or more optical signals is detected. In some embodiments, the method further comprises resecting the tumor. In some embodiments, the cancer is s breast cancer, head and neck squamous cell carcinoma (NHSCC), lung cancer, ovarian cancer, prostate cancer, bladder cancer, urethral cancer, esophageal cancer, colorectal cancer, brain cancer, or skin cancer. In some embodiments, the cancer is breast cancer, head and neck squamous cell carcinoma (NHSCC), esophageal cancer, pleural metastasis, kidney cancer, lymph node cancer, cervical cancer, pancreatic cancer, or colorectal cancer. In some embodiments, the pharmaceutical composition is administered at least 1 hour, at least 2 hours, at least 4 hours, at least 6 hours, at least 8 hours, at least 10 hours, at least 12 hours, at least 14 hours, at least 16 hours, at least 18 hours, at least 20 hours, at least 24 hours, at least 28 hours, at least 32 hours, at least 80 hours, at least 1 day, at least 2 days, at least 3 days, at least 4 days, at least 5 days, at least 6 days, at least 1 week, or at least 2 weeks prior to imaging the patient. In some embodiments, the pharmaceutical composition is administered from about 1 hour to about 32 hours, about 2 hours to about 32 hours, 16 hours to about 32 hours, about 20 hours to about 28 hours, about 1 hour to about 5 hours, or about 3 hours to about 9 hours prior to imaging the patient. In some embodiments, the pharmaceutical composition is administered as an injection or an infusion. In some embodiments, the pharmaceutical composition is administered as a single dose or multiple doses. In some embodiments, the method further comprises imaging the cancer patient comprises an intra-operative camera or an endoscopic camera. In some embodiments, the patient in need is a human patient. In some embodiments, the patient in need is a canine, feline, cow, horse, pig, or rabbit patient.

Other objects, features and advantages of the block copolymers, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIGS. 1A-1B display Phase 1a mean plasma concentration versus time following a single intravenous dose of the pharmaceutical composition comprising 0.1, 0.3, 0.5, 0.8, or 1.2 mg/kg of Compound 1. FIG. 1A displays the mean plasma concentration (LOG) versus time.

FIG. 1B displays the mean linear plasma concentration versus time.

FIG. 2 discloses the correlation between mean plasma concentration of the pharmaceutical composition at 10 minutes (C₁₀ₘ) and dose for Compound 1.

FIG. 3 discloses the correlation between mean AUC₀₋₂₄ₕᵣ and dose for Compound 1.

FIGS. 4A-4B display Phase 1b subject (patient) plasma concentration versus time following a single intravenous dose of the pharmaceutical composition comprising 1.2 mg/kg of Compound 1. FIG. 4A displays mean plasma concentration doses for patient plasma concentration (Log) versus time. FIG. 4B displays patient plasma concentrations (Linear) versus time.

FIGS. 5A-5B display the Phase 1a and Phase 1b mean plasma concentration versus time following a single intravenous dose of the pharmaceutical composition comprising 0.1, 0.3, 0.5, 0.8, or 1.2 mg/kg of Compound 1. FIG. 5A displays the Phase 1a and Phase 1b mean plasma concentration (Log) versus time by dose. FIG. 5B displays the Phase 1a and Phase 1b mean plasma concentration (Linear) versus time by dose.

FIG. 6 displays Phase 1a and Phase 1b mean (±SD) plasma concentration at 10 min versus dose of Compound 1.

FIG. 7 displays Phase 1a and Phase 1b mean (±SD) AUC₀₋₂₄ₕᵣ versus dose.

FIGS. 8A-8J display mean plasma concentration of Compound 1 by tumor type. FIG. 8A displays Phase 1a (1.2 mg/kg) and Phase 1b mean plasma concentrations (Log) versus time by tumor type; FIG. 8B displays Phase 1a (1.2 mg/kg) and Phase 1b mean plasma concentrations (Linear) versus time by tumor type; FIG. 8C displays Phase 1a (1.2 mg/kg) and Phase 1b patient plasma concentrations (Log) versus time in breast cancer; FIG. 8D displays Phase 1a (1.2 mg/kg) and Phase 1b patient plasma concentrations (Log) versus time in colorectal cancer tumors; FIG. 8E displays Phase 1a (1.2 mg/kg) and Phase 1b patient plasma concentrations (Log) versus time in esophageal cancer tumors; FIG. 8F displays Phase 1a (1.2 mg/kg) and Phase 1b individual plasma concentrations (Log) versus time in head and neck (HNSCC) tumors; FIG. 8G displays Phase 1a (1.2 mg/kg) and Phase 1b patient plasma concentrations (Linear) versus time in breast cancer tumors; FIG. 8H displays Phase 1a (1.2 mg/kg) and Phase 1b patient plasma concentrations (Linear) versus time in colorectal cancer tumors; FIG. 8I displays Phase 1a (1.2 mg/kg) and Phase 1b patient plasma concentrations (Linear) versus time in esophageal cancer tumors; FIG. 8J displays Phase 1a (1.2 mg/kg) and Phase 1b patient plasma concentrations (Linear) versus time in HNSCC tumors.

FIGS. 9A-9B display intraoperative images from three patients dosed with 0.5 mg/kg (FIG. 9A) and at 1.2 mg/kg (FIG. 9B) respectively of Compound 1 and imaged using a NOVADAQ SPY Elite camera. Left column displays white light images and right hand column displays fluorescent images.

FIGS. 10A-10B display postoperative specimen taken with images for 3 patients in dosed with 0.5 mg/kg (FIG. 10A) and at 1.2 mg/kg (FIG. 10B) of Compound 1 respectively using a LI-COR Pearl camera.

FIGS. 11A-11B display contrast-to-noise (CNR, FIG. 11A) and tumor-to-background (TBR, FIG. 11B) fluorescence intensity contrast ratio.

FIGS. 12A-12B display postoperative mean fluorescence intensity of histology-confirmed tumor and normal tissue of specimens (formalin-fixed (FF) or fresh) versus dose (FIG. 12A) and postoperative mean fluorescence intensity of histology-confirmed tumor and normal tissue versus initial plasma concentration (FIG. 12B).

FIGS. 13A-13B display CNR (FIG. 13A) and TBR (FIG. 13B) fluorescence ratios respectively calculated using the postoperative mean fluorescence intensity obtained from histology-confirmed tumor and normal regions of the pathologist selected bread loaf slices for all 15 patients at 5 dose levels (formalin-fixed (FF) or fresh).

FIG. 14 shows study design. Intravenous administration of Compound 1 was performed 24 hours (±8 h) prior to surgery. Ten days of safety assessments (laboratory, PK, ECGs) followed, adverse events were monitored to day 17 (a). During surgery, intraoperative images were obtained prior to incision and after excision of the surgical cavity (b). Immediately after excision the specimen was imaged for the presence of a positive surgical margin (c). Fluorescence images were obtained during all the standard pathology processing phases (d, e), and the HE slices were correlated with the standard histopathology slices (f-h). ECG electrocardiogram, H/E hematoxylin eosin, SOC standard of care.

FIG. 15 shows fluorescence images of different tumor tissue slices. Head and neck squamous cell cancer of the tongue (a-f); breast cancer (g-l); esophageal cancer (m-r)l colorectal cancer (s-x). The tumor is delineated as a solid black line in the H/E slices (c, i, o, u). The mean fluorescence intensity (MFI) of the tumor tissue and the non-tumor tissue slices per tumor type is depicted (y). The dots represent the MFI of single tissue slices (about 3 per subject) from the 1.2 mg/kg cohort. HNSCC, 7 subjects, P<0.0001; BC, 5 subjects, P=0.0001; EC, 3 subjects, P=0.0010; and Wilcox test, two-sided. CRC, 3 subjects, no statistics performed due to the availability of only three data points.

FIG. 16 displays Compound 1 fluorescence results with postoperative tissue specimens in different tumor types. The image shows representative examples of a head and neck squamous cell carcinoma of the tongue from a subject with a negative surgical margin. In- and ex vivo visualization of fluorescence in tumor (a, c, g, i) with no fluorescent signal in the surgical cavity or at the surgical resection (b, h, d, j). Correlation of fluorescent signals on a tissue slice with the histology (e, k, f) with a tumor-negative surgical margin of 6.4 mm. Representative example of breast cancer surgery (i.e. a lumpectomy) with a tumor-positive surgical margin (1, m, n, o). Fluorescence is detected at the ventral surgical margin both in vivo and immediately after excision (r, s, t, u) which corresponds with the fluorescence localization on the tissue slice (p, v) and the final histopathology (q). The tumor is delineated as a solid black line on the H/E slices (f, q). H/E hematoxylin eosin, SOC standard of care.

FIG. 17 displays clinically relevant images for HNSCC and BC. (a-c) displays intraoperatively detected peritoneal metastasis (PM). (d-f) display additional tumor lesion detected in the surgical cavity after a Head and Neck Squamous Cell Carcinoma (HNSCC) resection of the mandible. (g-i) show a false positive fluorescent lesion from salivary gland tissue. (j-o) show additional satellite metastases of the primary tumor lesion were detected in tow BC subjects and confirmed by final histopathological examination. (p-r) show an additional primary tumor lesion was detected on a fresh tissue slice from a BC subject showing triple negative breast cancer which was not detected before and during surgery. (c, f, 1, o, r) show that the tumor is delineated as a solid black line in the H/E slides. (i) shows that the false positive contained no viable tumor tissue.

FIGS. 18A-18B describes fluorescence microscopy to confirm tumor-specific activation of Compound 1. FIG. 18A displays florescence microscopy performed ex vivo after spraying Compound 1 onto tissue sections of freshly frozen HNSCC specimen directly after excision. DAPI was applied for nuclear staining (a) and Compound 1 for fluorescence visualization (b). A sharp delineation of fluorescence between the tumor and stromal tissue (c) was observed and correlated with corresponding histopathology tissue sections tainted with hematoxylin and eosin (d). FIG. 18B shows pH-dependent activation of Compound 1 in human plasma. Increasing amounts of Compound 1 were added to human plasma which did not show any increase in fluorescence. When the experiment was repeated with HCl to supply protons to the plasma, there was an increase in fluorescence with the addition of increasing amounts of intact Compound 1 suggesting that acidosis was activating the Compound 1 and thus the fluorescence in a dose-dependent manner. RFU: relative fluorescence units.

FIG. 19 correlates the fluorescent surgical margin assessment with final histopathology results. Intraoperative assessment of the surgical margin during fluorescence-guided surgery can be done either by intraoperative fluorescence imaging of the surgical cavity or fluorescence imaging of the excised specimen at the back-table. The final histopathology is correlated with the fluorescence images of breast cancer subjects (a) and head and neck squamous cell carcinoma subjects (b).

FIG. 20 describes dose-independent mean fluorescence intensity separation between tumor tissue and non-tumor tissue. Tumor and non-tumor tissue mean fluorescence intensities (MFI) from the 0.1 mg per kg cohort, P=0.0005 (a); 0.3 mg per kg cohort, P=0.0078 (b); 0.5 mg per kg cohort, P=0.0020 (c); 0.8 mg per kg cohort, P=0.0078 (d); and 1.2 mg per kg cohort, P<0.0001, Wilcoxon test, two-sided (e). The dots represent the MFI of single tissue slices. The receiver-operator characteristics curve is based on the calculated MFI of the tumor and normal tissues from the 1.2 mg per kg dose cohort, P<0.0001; area under the curve 0.9875, n=59, with a confidence interval of 95% using Wilson/Brown method (f). ROC receiver operators curve, AUC area under the curve. **P≤0.01; ***P≤0.001; ****P≤0.0001.

FIG. 21 shows in vivo imaging using Compound 1 fluorescence. Representative examples of in vivo imaging data using Compound 1 fluorescence. A large tongue carcinoma with a central necrotic ulcer was in vivo visualized using Compound 1 (a). A cancer located at the right mandible/floor of mouth was in vivo visualized using Compound 1 (b). A large tongue carcinoma with a central necrotic ulcer was visualized using Compound 1 (c). A colorectal carcinoma with extensive peritoneal metastases was in vivo visualized using Compound 1 (d).

FIG. 22 shows fluorescent imaging of breast cancer and HNSCC tumors 3-9 hours and 1-5 hours post dosing with Compound 1. Images shown with SPY Elite and VisionSense cameras.

FIG. 23 demonstrates that Compound 1 fluoresced intraoperatively in prostate cancer through thin prostatic capsule using Da Vinci Firefly camera with updated software and hardware. No fluorescence was detected in the surgical bed consistent with negative margins confirmed through pathology.

FIG. 24 demonstrates Compound 1 fluorescence in ovarian cancer (recurrent at vaginal cuff) using VisionSense camera Pre-excision in vivo imaging was performed after 6±3 hours of Compound 1 dosing at 3 mg/kg.

FIG. 25 shows Compound 1 fluorescence on bread loaf slide (BLS) tissue specimens corresponding to pathology-confirmed tumor areas.

FIG. 26 shows Compound 1 fluorescence was verified in all visible BC and HNSCC tumors with 3-5 hour dose schedule timing using SPY Elite camera.

FIG. 27 shows mast cell tumor resected from dog-patient. Representative white light (left) and fluorescence images (right) of a resected mast cell tumor from dog-patient after Compound 1 administration.

FIG. 28 shows representative images from soft tissue sarcoma. The white light image of the mast cell tumor is evident in (A) and can also be easily observed intra-operatively in (B) using a custom NIR camera above prior to excision. The white light photo of the resected tumor with tissue margin is shown in (C), and the corresponding fluorescent image of the resected tumor as imaged by the LI-COR Pearl is overlapped with white light image to show the colocalization of the fluorescence with white light anatomy (D). Histopathology confirmed the malignancy of the resected tissue.

FIG. 29 shows representative images from dog-patient with osteosarcoma. (A) shows the white light photo of the lesion on the amputated leg; the green and black dotted lines indicate the location of the normal and cancerous tissue cross-sections, respectively. (B) shows the NIR tumor image taken using a Hamamatsu PDE NIR camera. (C) shows a white light photo of the cross-sections from the normal (left, smaller) and cancerous tissues (right, larger) as described in (A). (D) shows the NIR image of the cross-sections of the same normal (non-fluorescent) and cancerous (fluorescent tissues) shown in (C).

FIG. 30 shows representative images from a dog-patient with a soft tissue sarcoma. A white light image of the resected soft tissue sarcoma with margins is shown on the left side of the figure and the fluorescent image (overlapping with white light) of the tumor tissues is shown on the right side of the figure. Histopathology confirmed the malignancy of the resected tissue.

FIG. 31 shows images from dog-patient with a primary soft tissue pinna sarcoma. White light images of the soft tissue pinna sarcoma are shown on the top left and lower left panels. A NIR image taken post-amputation of the ear using the Hamamatsu PDE shows the tumor fluorescing through the skin (lower middle panel). The ear was also imaged using the LI-COR system showing the remained fluorescence, after performing a core punch biopsy (lower right and inset images, respectively). Histopathology analysis of the punch biopsy confirmed the malignancy of the tissue.

FIG. 32 shows images from a dog-patient with a primary soft tissue sarcoma and a distal tumor affected lymph node. The white light image in the upper left panel shows the primary soft tissue sarcoma. During surgical removal of this mass, a popliteal lymph node was observed to be enlarged (upper right-most panel) and this was removed and imaged using the LI-COR (center-most panel). The fluorescent images show the transected lymph node to be diseased and this was corroborated by histopathology.

### DETAILED DESCRIPTION OF THE DISCLOSURE

Some embodiments provided herein describe a micelle-based, fluorescent imaging agent. In some embodiments, the micelles comprise a diblock copolymer of polyethylene glycol (PEG) and a dibuthylamino substituted polymethylmethacrylate (PMMA) covalently conjugated to indocyanine green (ICG) through NHS chemistry on 2-Aminoethyl methacrylate hydrochloride monomers. In some embodiments, the PEGs comprise the shell or surface of the stable micelle. In some embodiments, the micellar size is <100 nm.

### I. Compounds

In some embodiments, provided herein is a block copolymer having the structure of Formula (II), or a pharmaceutically acceptable salt, solvate, or hydrate thereof:

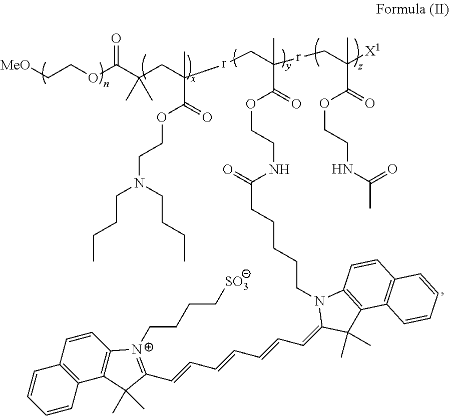

wherein:
X¹ is a halogen, —OH, or —C(O)OH;
n is 90-140;
x is 50-200;
y is 0-3; and
z is 0-3.

In some embodiments, the block copolymer of Formula (II) is a compound. In some embodiments, the block copolymer of Formula (II) is a diblock copolymer. In some embodiments, the block copolymer of Formula (II) is a block copolymer comprising a hydrophilic polymer segment and a hydrophobic polymer segment.

The hydrophilic polymer segment comprises poly(ethylene oxide) (PEO). In some embodiments, the hydrophilic polymer segment is about 2 kDa to about 10 kDa in size. In some embodiments, the hydrophilic polymer segment is about 2 kDa to about 5 kDa in size. In some embodiments, the hydrophilic polymer segment is about 3 kDa to about 8 kDa in size. In some embodiments, the hydrophilic polymer segment is about 4 kDa to about 6 kDa in size. In some embodiments, the hydrophilic polymer segment is about 5 kDa in size.

In some embodiments, the block copolymer comprises a hydrophobic polymer segment. In some embodiments, the hydrophobic polymer segment comprises a tertiary amine. In some embodiments, the hydrophobic polymer segment comprises:

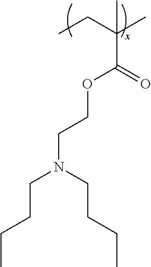

wherein x is about 50-200 in total. In some embodiments, x is about 60-150. In some embodiments, x is an integer between about 60 to about 150. In some embodiments, the hydrophilic segment comprises a dibutyl amine.

In some embodiments, there are n repeating polyethylene oxide repeating units. In some embodiments, n is 90-140. In some embodiments, n is 95-130. In some embodiments, n is 100-120. In some embodiments, n is 100, 101, 102, 103, 104, 105, 106, 107, 108, 109, 110, 111, 112, 113, 114, 115, 116, 117, 118, 119, or 120. In some embodiments, n is 114. In some embodiments, n is 113.

In some embodiments, y is 0-3. In some embodiments, y is 0.5-2.5. In some embodiments, y is 1.5-2.5. In some embodiments, y is 0.5-1.5. In some embodiments, y is 0.5, 1, 1.5, 2, 2.5, or 3. In some embodiments, y is 1, 2, or 3. In some embodiments, y is 0.5. In some embodiments, y is 1.5. In some embodiments, y is 0.

In some embodiments, z is 0-3. In some embodiments, z is 1.5-2.5. In some embodiments, z is 1, 1.5, 2, 2.5, or 3. In some embodiments, z is 1, 2, or 3. In some embodiments, z is 1.5. In some embodiments, z is 0.

In some embodiments, the copolymer block units (x, y, and z) can occur in any order or configuration. In some embodiments, x, y, and z occur sequentially as described in Formula (II).

In certain embodiments, the block copolymer comprises a fluorescent dye conjugated through an amine. In some embodiments, the fluorescent dye is a pH-insensitive dye. In some embodiments, the fluorescent dye is a cyanine dye or a derivative thereof. In some embodiments, the fluorescent dye is indocyanine green (ICG). Indocyanine green (ICG) is used in medical diagnostics.

In some embodiments, the block copolymer is not conjugated to a fluorescent dye or a derivative thereof. In some embodiments, the block copolymer is not conjugated to indocyanine green (ICG).

In some embodiments, the block copolymer of Formula (II) is poly(ethyleneoxide)-b-poly(dibutylaminoethyl methacrylate-r-aminoethylmethylacrylate hydrochloride) copolymer indocyanine green and acetic acid conjugate. In some embodiments, the block copolymer of Formula (II) is PEO₉₀₋₁₄₀-b-P(DBA₆₀₋₁₅₀-r-ICG₀₋₃-r-AMA₀₋₃), (Compound 1).

In some embodiments, X¹ is a terminal group. In some embodiments, the terminal capping group is the product of an atom transfer radical polymerization (ATRP) reaction. In some embodiments, X¹ is a halogen. In some embodiments, X¹ is Br. In some embodiments, X¹ is —OH. In some embodiments, X¹ is an acid. In some embodiments, X¹ is —C(O)OH. In some embodiments, X¹ is H.

The term “r” denotes a connection between different block copolymer units/segments (e.g., represented by x, y, and z). In some embodiments, each r is independently a bond connecting carbon atoms of the units/segments, or an alkyl group —(CH₂)ₙ— wherein n is 1 to 10. In some embodiments, the copolymer block segments/units (e.g., represented by x, y, and z) can occur in any order, sequence, or configuration. In some embodiments, the copolymer block units occur sequentially as described in Formula (II).

In some embodiments, the block copolymer of Formula (II) has the structure of Formula (II-a), or a pharmaceutically acceptable salt, solvate, or hydrate thereof:

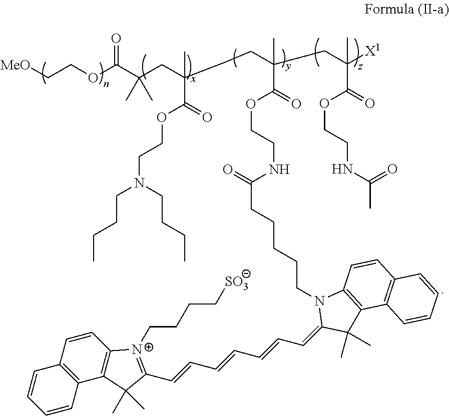

In some embodiments, the block copolymer of Formula (II) is in the form of a micelle or nanoparticle. The size of the micelles will typically be in the nanometer scale (i.e., between about 1 nm and 1 μm in diameter). In some embodiments, the micelle has a size of about 10 to about 200 nm. In some embodiments, the micelle has a size of about 20 to about 100 nm. In some embodiments, the micelle has a size of about 30 to about 50 nm. In some embodiments, the micelle has a diameter less than about 1 μm. In some embodiments, the micelle has a diameter less than about 100 nm. In some embodiments, the micelle has a diameter less than about 50 nm.

In another aspect, provided herein is a pH responsive composition comprising one or more block copolymers of Formula (II).

In some embodiments, the pH responsive composition has a pH transition point and an emission spectrum. In some embodiments, the pH transition point is between 4-8 or between 6-7.5. In some embodiments, the pH transition point is between 4.8-5.5. In some embodiments, the pH transition point is at about 4.8, 4.9, 5.0, 5.1, 5.2, 5.3, 5.4, or 5.5. In some embodiments, the pH transition point is 4.8. In some embodiments, the pH transition point is 4.9. In some embodiments, the pH transition point is 5.0. In some embodiments, the pH transition point is 5.1. In some embodiments, the pH transition point is 5.2. In some embodiments, the pH transition point is 5.3. In some embodiments, the pH transition point is 5.4. In some embodiments, the pH transition point is 5.5.

In some embodiments, the pH responsive composition has an emission spectrum between 700-900 nm. In some embodiments, the pH responsive composition has an emission spectrum between 750-800 nm. In some embodiments, the pH responsive composition has an emission spectrum between 750-850 nm.

In some embodiments, the pH responsive composition has a pH transition range (ΔpH_{10-90%}). In some embodiments, the pH responsive composition has a pH transition range of less than 1 pH unit. In some embodiments, the pH responsive composition has a pH transition range of less than 0.25 pH unit. In some embodiments, the pH responsive composition has a pH transition range of less than 0.15 pH unit.

In some embodiments, the composition has a fluorescence activation ratio. A fluorescence activation ratio is defined as: the ratio of the normalized fluorescence intensity from the formulation in buffers with pH<pHₜ (transitional pH of the formulation) to the normalized fluorescence intensity from the formulation in buffers with pH>pHₜ. In some embodiments, the fluorescence activation ratio is greater than 25. In some embodiments, the fluorescence activation ratio is greater than 50.

### II. Pharmaceutical Compositions

The pharmaceutical compositions disclosed herein, comprise one or more pH-responsive micelles and/or nanoparticles that comprise block copolymers and the fluorescent dye indocyanine green. The block copolymer comprises a hydrophilic polymer segment and a hydrophobic polymer segment wherein the hydrophobic polymer segment comprises an ionizable amine group to render pH sensitivity. This pH sensitivity is exploited to provide pharmaceutical compositions suitable as diagnostic tool for imaging (e.g. to aid in tumor resection and staging).

In an aspect, provided herein is a pharmaceutical composition comprising a micelle, wherein the micelle comprises

1) one or more block copolymers having the structure of Formula (II), or a pharmaceutically acceptable salt, solvate, or hydrate thereof:

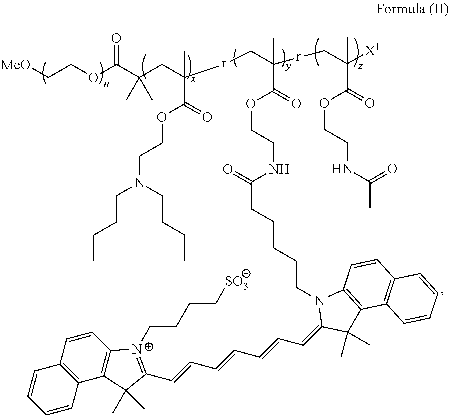

wherein:
X¹ is a halogen, —OH, or —C(O)OH;
n is 90-140;
x is 50-200;
y is 0-3; and
z is 0-3; and
   2) a stabilizing agent.

In some embodiments, the pharmaceutical composition comprises a micelle, wherein the micelle comprises one or more block copolymers having the structure of Formula (II), or a pharmaceutically acceptable salt, solvate, or hydrate thereof. In some embodiments, the block copolymer of Formula (II), or a pharmaceutically acceptable salt, solvate, or hydrate thereof, is a micelle-based fluorescent imaging agent. In some embodiments, the block copolymer of Formula (II) is poly(ethyleneoxide)-b-poly(dibutylaminoethyl methacrylate-r-aminoethylmethylacrylate hydrochloride) copolymer indocyanine green and acetic acid conjugate. In some embodiments, the block copolymer of Formula (II) is PEO₉₀₋₁₄₀-b-P(DBA₆₀₋₁₅₀-r-ICG₀₋₃-r-AMA₀₋₃), (Compound 1). In some embodiments, the block copolymer is a copolymer capable of forming a micelle or nanoparticle.

In some embodiments, the pharmaceutical composition comprises about 1 mg/mL to about 5 mg/mL of the block copolymer of Formula (II), or a pharmaceutically acceptable salt, solvate, or hydrate thereof. In some embodiments, the pharmaceutical composition comprises about 1 mg/mL, about 1.5 mg/mL, about 2 mg/mL, about 2.5 mg/mL, about 3 mg/mL, about 3.5 mg/mL, about 4 mg/mL, about 4.5 mg/mL, or about 5 mg/mL of the block copolymer of Formula (II).

In some embodiments, the pharmaceutical composition comprises about 3.0 mg/mL of the block copolymer of Formula (II), or a pharmaceutically acceptable salt, solvate, or hydrate thereof.

In some embodiments, the pharmaceutical composition comprises about 0.1 mg/kg to about 8 mg/kg of the block copolymer of Formula (II), or a pharmaceutically acceptable salt, solvate, or hydrate thereof. In some embodiments, the pharmaceutical composition comprises about 0.5 mg/kg to about 7 mg/kg of the block copolymer of Formula (II), or a pharmaceutically acceptable salt, solvate, or hydrate thereof. In some embodiments, the pharmaceutical composition comprises about 0.1 mg/kg to about 3 mg/kg of the block copolymer of Formula (II), or a pharmaceutically acceptable salt, solvate, or hydrate thereof. In some embodiments, the pharmaceutical composition comprises from about 0.1 to about 1.2 mg/kg of the block copolymer of Formula (II), or a pharmaceutically acceptable salt, solvate, or hydrate thereof.

In some embodiments, the pharmaceutical composition comprises about 0.5 mg/kg, 1 mg/kg, 2 mg/kg, 3 mg/kg, 4 mg/kg, 5 mg/kg, 6 mg/kg, or 7 mg/kg of the block copolymer of Formula (II), or a pharmaceutically acceptable salt, solvate, or hydrate thereof. In some embodiments, the pharmaceutical composition comprises about 0.1 mg/kg, 0.3 mg/kg, 0.5 mg/kg, 0.8 mg/kg, 1 mg/kg, 1.2 mg/kg, 1.4 mg/kg, 1.6 mg/kg, 1.8 mg/kg, 2 mg/kg, 2.5 mg/kg, or 3 mg/kg of the block copolymer of Formula (II), or a pharmaceutically acceptable salt, solvate, or hydrate thereof. In some embodiments, the pharmaceutical composition comprises about 0.1 mg/kg, 0.3 mg/kg, 0.5 mg/kg, 0.8 mg/kg, 1 mg/kg, or 1.2 mg/kg of the block copolymer of Formula (II), or a pharmaceutically acceptable salt, solvate, or hydrate thereof. In some embodiments, the pharmaceutical composition comprises about 0.1 mg/kg of the block copolymer of Formula (II). In some embodiments, the pharmaceutical composition comprises about 0.3 mg/kg of the block copolymer of Formula (II). In some embodiments, the pharmaceutical composition comprises about 0.5 mg/kg of the block copolymer of Formula (II). In some embodiments, the pharmaceutical composition comprises about 0.8 mg/kg of the block copolymer of Formula (II). In some embodiments, the pharmaceutical composition comprises about 1 mg/kg of the block copolymer of Formula (II). In some embodiments, the pharmaceutical composition comprises about 1.2 mg/kg of the block copolymer of Formula (II). In some embodiments, the pharmaceutical composition comprises about 1.4 mg/kg of the block copolymer of Formula (II). In some embodiments, the pharmaceutical composition comprises about 1.6 mg/kg of the block copolymer of Formula (II). In some embodiments, the pharmaceutical composition comprises about 1.8 mg/kg of the block copolymer of Formula (II). In some embodiments, the pharmaceutical composition comprises about 2 mg/kg of the block copolymer of Formula (II). In some embodiments, the pharmaceutical composition comprises about 2.5 mg/kg of the block copolymer of Formula (II). In some embodiments, the pharmaceutical composition comprises about 3 mg/kg of the block copolymer of Formula (II). In some embodiments, the pharmaceutical composition comprises about 3.5 mg/kg of the block copolymer of Formula (II). In some embodiments, the pharmaceutical composition comprises about 4 mg/kg of the block copolymer of Formula (II). In some embodiments, the pharmaceutical composition comprises about 5 mg/kg of the block copolymer of Formula (II). In some embodiments, the pharmaceutical composition comprises about 6 mg/kg of the block copolymer of Formula (II). In some embodiments, the pharmaceutical composition comprises about 7 mg/kg of the block copolymer of Formula (II).

In some embodiments of the pharmaceutical compositions disclosed herein, the block copolymer of Formula (II), or pharmaceutically acceptable salt, solvate, or hydrate thereof, is substantially pure. In some embodiments of the pharmaceutical compositions disclosed herein, the block copolymer of Formula (II), or a pharmaceutically acceptable salt, solvate, or hydrate thereof, is substantially free of impurities. In some embodiments of the pharmaceutical compositions disclosed herein, substantially free of impurities is defined as less than about 10%, about 5%, about 3%, about 1%, about 0.5%, about 0.1%, or about 0.05% content of impurities. In some embodiments of the pharmaceutical compositions disclosed herein, substantially free of impurities is defined as less than about 1% content of impurities. In some embodiments of the pharmaceutical compositions disclosed herein, substantially free of impurities is defined as less than about 0.5% content of impurities. In some embodiments of the pharmaceutical compositions disclosed herein, substantially free of impurities is defined as less than about 0.1% content of impurities.

In some embodiments of the pharmaceutical compositions disclosed herein, the block copolymer of Formula (II), or a pharmaceutically acceptable salt, solvate, or hydrate thereof, is at least about 90%, about 95%, about 98%, or about 99% pure.

In some embodiments of the pharmaceutical compositions disclosed herein, the block copolymer of Formula (II), or a pharmaceutically acceptable salt, solvate, or hydrate thereof, is at least about 99.1%, about 99.2%, about 99.3%, about 99.4%, about 99.5%, about 99.6%, about 99.7%, about 99.8%, about 99.9%, or about 100% pure.

The term “stabilizing agent” is meant to mean an agent that, when added to a biologically active material will prevent or delay the loss of the material's biological activity over time as compared to when the material is stored in the absence of the stabilizing agent. Some of these additives have been found to extend the shelf life of a biologically active material to many months or more when stored at ambient temperature in an essentially dehydrated form. Additionally, a variant of cryoprotective additives and agents have been used as excipients to help with and preserve the biological activity when biological materials are dried or frozen. Protective substances are water-soluble saccharides such as monosaccharides, disaccharides, trisaccharide, water soluble polysaccharides, sugar alcohols, polyols, or mixtures of these. Examples of monosaccharides, disaccharide and trisaccharide include but are not limited to glucose, mannose, glyceraldehyde, xylose, lyxose, talose, sorbose, ribulose, xylulose, galactose, fructose, sucrose, trehalose, lactose, maltose, and raffinose. Among water-soluble polysaccharides include certain water-soluble starches and celluloses. Examples of sugar alcohols are glycerol. Other substances that function as stabilizing agents include for example amino acids such as arginine, and proteins such as albumin.

In some embodiments, pharmaceutically acceptable excipient is a cryoprotective agent or a stabilizing agent. In some embodiments, pharmaceutically acceptable excipient is a stabilizing agent. In some embodiments, the stabilizing agent is a sugar, a sugar derivative, a detergent, and a salt.

In some embodiments, the stabilizing agent is a monosaccharide, disaccharide, trisaccharide, water soluble polysaccharide, sugar alcohol, or polyol, or combination thereof. In some embodiments, the stabilizing agent is fructose, galactose, glucose, lactose, sucrose, trehalose, maltose, mannitol, sorbitol, ribose, dextrin, cyclodextrin, maltodextrin, raffinose, or xylose, or a combination thereof. In some embodiments, the stabilizing agent is trehalose. In some embodiments, the stabilizing agent is trehalose dihydride.

In some embodiments, the pharmaceutical composition comprises from about 0.5% w/v to about 25% w/v, from about 1% to about 20% w/v, from about 5% to about 15% w/v, from about 6% to about 13% w/v, from about 7% to about 12% w/v, or from about 8% to about 11% w/v of the stabilizing agent. In some embodiments, the pharmaceutical composition comprises from about 7% to about 12% w/v of the stabilizing agent. In some embodiments, the pharmaceutical composition comprises from about 8% to about 11% w/v of the stabilizing agent.

In some embodiments, the pharmaceutical composition comprises about 5% w/v, about 6% w/v, about 7% w/v, about 8% w/v, about 9% w/v, about 10% w/v, about 11% w/v, about 12% w/v, about 13% w/v, about 14% w/v, or about 15% w/v of the stabilizing agent. In some embodiments, the pharmaceutical composition comprises about 9% w/v of the stabilizing agent. In some embodiments, the pharmaceutical composition comprises about 10% w/v of the stabilizing agent. In some embodiments, the pharmaceutical composition comprises about 11% w/v of the stabilizing agent. In some embodiments, the pharmaceutical composition comprises about 12% w/v of the stabilizing agent. In some embodiments, the pharmaceutical composition comprises about 13% w/v of the stabilizing agent. In some embodiments, the pharmaceutical composition comprises about 14% w/v of the stabilizing agent. In some embodiments, the pharmaceutical composition comprises about 15% w/v of the stabilizing agent.

In some embodiments, the pharmaceutical composition further comprises a liquid carrier. In some embodiments, the liquid carrier is an aqueous solution. In some embodiments, the liquid carrier is selected from sterile water, sterile water for injection (SWFI), normal saline, half normal saline, dextrose (such as aqueous dextrose; e.g. 5% dextrose in water D5W), or ringers lactate solution (RL) or combination therein (such as 50% dextrose and 50% normal saline). In some embodiments, the liquid carrier is selected from sterile water.

In some embodiments, the pharmaceutical composition comprises at least about 3 mg/mL of a block copolymer having the structure of Formula (II), or a pharmaceutically acceptable salt, solvate, or hydrate thereof:

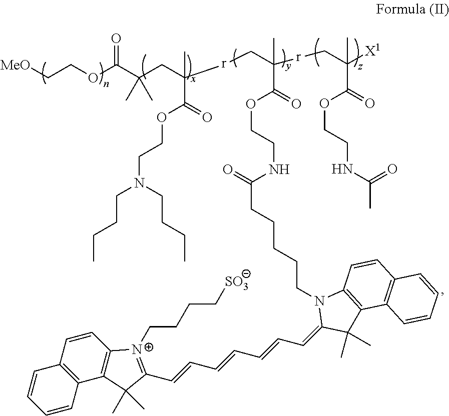

wherein:
X¹ is Br;
n is 90-140;
x is 60-150;
y is 0-3; and
z is 0-3; and
about 10% w/v trehalose in water.

The pharmaceutical compositions of the present disclosure can be formulated to be compatible with the intended method or route of administration; exemplary routes of administration are set forth herein.

In some embodiments, the pharmaceutical composition disclosed herein is in a form for dosing or administration by oral, intravenous (I.V.), intramuscular, subcutaneous, intratumoral, or intradermal injection. In some embodiments, the pharmaceutical composition is formulated for oral, intramuscular, subcutaneous, or intravenous administration. In some embodiments, the pharmaceutical composition is formulated for intratumoral administration. In some embodiments, the pharmaceutical composition is formulated for intravenous administration. In some embodiments, the pharmaceutical composition is formulated as an aqueous solution or suspension for intravenous (I.V.) administration. In some embodiments, the pharmaceutical composition is formulated to administer as a single dose. In some embodiments, the pharmaceutical composition is formulated to administer as multiple doses. In some embodiments, the pharmaceutical composition disclosed herein is formulated to administer as a bolus by I.V.

In some embodiments of the pharmaceutical composition, wherein the form is an I.V. dosage form, the pH is from about 3.5 to about 8.5. In some embodiments, the pH of the I.V. dosage is about 3.5, 4.0, 4.5, 5.0, 5.5, 6.0, 6.5, 7.0, 7.5, 8.0, or 8.5.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanthin and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation.

Dispersible powders and granules suitable for preparation of an aqueous suspension by the addition of water provide the active ingredient in admixture with a dispersing or wetting agent, and optionally one or more suspending agents and/or preservatives. Suitable dispersing or wetting agents and suspending agents are exemplified herein.

The pharmaceutical compositions of the present invention may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanthin; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of a block copolymer of Formula (II) or a pharmaceutically acceptable salt, solvate, or hydrate thereof, and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate-buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer; N-(2-Hydroxyethyl)piperazine-N′-(2-ethanesulfonic acid) (HEPES); 2-(N-Morpholino)ethanesulfonic acid (MES); 2-(N-Morpholino)ethanesulfonic acid sodium salt (MES); 3-(N-Morpholino)propanesulfonic acid (MOPS); and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampule, syringe, or autoinjector, whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time-delay material such as glyceryl monostearate or glyceryl stearate alone, or in combination with a wax, may be employed. Any drug delivery apparatus may be used to deliver a block copolymer of Formula (II), or a pharmaceutically acceptable salt, solvate, or a hydrate thereof, including implants (e.g., implantable pumps) and catheter systems, slow injection pumps and devices, all of which are well known to the skilled artisan.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor® EL (BASF, Parsippany, N.J.) or phosphate buffered saline (PBS), ethanol, polyol (e.g., glycerol, propylene glycol, and liquid polyethylene glycol), sterile water for injection (SWFI), D5W, and suitable mixtures thereof. In addition, sterile fixed oils are conventionally employed as a solvent or suspending medium; for this purpose, any bland fixed oil may be employed, including synthetic mono- or diglycerides. Moreover, fatty acids, such as oleic acid, find use in the preparation of injectables. Prolonged absorption of particular injectable formulations can be achieved by including an agent that delays absorption (e.g., aluminum monostearate or gelatin).

### III. Methods of Use

In some embodiments, the pharmaceutical compositions described herein are used in a pH responsive composition. In some embodiments, the pH responsive compositions are used to image physiological and/or pathological processes that involve changes to intracellular or extracellular pH (e.g. acidic pH of a cancerous tumor). In some embodiments, the pharmaceutical compositions micelles described herein are useful for the detection of primary and metastatic tumor tissues (including peritoneal metastases and lymph nodes), leading to reduced tumor recurrence and re-operation rates. In some embodiments, the pH-sensitive imaging agents can detect a tumor from the surrounding normal tissue with high tumor contrast to background fluorescent response ratio (CNR and TBR).

Aerobic glycolysis, known as the Warburg effect, in which cancer cells preferentially uptake glucose and convert it into lactic acid or other acids, occurs in all solid cancers. Lactic acid or other acids preferentially accumulates in the extracellular space due to monocarboxylate transporters or other transporters. The resulting acidification of the extra-cellular space promotes remodeling of the extracellular matrix for further tumor invasion and metastasis.

Real-time fluorescence imaging during surgery will help surgeons to detect or delineate tumor versus normal tissue or metastatic disease such as from diseased lymph nodes, with the goal of achieving negative margins and complete tumor resection and to aid in staging. These improved surgical outcomes translate to significant clinical benefits such as reduced tumor recurrence and re-operation rates, avoidance of unnecessary surgeries, preservation of function and cosmesis.

Another key objective of cancer surgery is to assist in pathological staging for treatment decisions. Due to occult nodal metastasis, lymph node status is a key component of cancer staging. Elective comprehensive regional nodal dissection is standard of care (SOC) for head and neck cancer because simple node sampling during surgery underestimates nodal metastases. With colorectal cancer for example, up to 25% of “node-negative” patients die from relapse and metastases indicating the presence of residual occult disease, and lymph node metastasis adds prognostic value especially for stage II colorectal patients. Accurately detecting nodal metastases for these patients can lead to upstaging and adjuvant treatment intensification, better matching therapy to disease.

Thus, techniques that can selectively and accurately improve the intraoperative visualization of tumor margins, occult tumors, and tumor-positive lymph nodes and other metastatic disease would potentially improve the completeness of surgical resection, the appropriateness of adjuvant therapy selection, pathological staging and oncologic outcomes for patients with solid tumors.

Some embodiments provided herein, describe block copolymers that form micelles at physiologic pH (7.35-7.45). In some embodiments, the block copolymers described herein are conjugated to ICG dyes. In some embodiments, the micelle has a molecular weight of greater than 2×10⁷ Daltons. In some embodiments, the micelle has a molecular weight of ˜2.7×10⁷ Daltons. In some embodiments, the ICG dyes are sequestered within the micelle core at physiologic pH (7.35-7.45) (e.g., during blood circulation) resulting in fluorescence quenching. In some embodiments, when the micelle encounters an acidic environment (e.g., tumor tissues), the micelles dissociate into individual compounds with an average molecular weight of about 3.7×10⁴ Daltons, allowing the activation of fluorescence signals from the ICG dye, causing the acidic environment (e.g. tumor tissue) to specifically fluoresce. In some embodiments, the micelle dissociates at a pH below the pH transition point (e.g. acidic state of the tumor microenvironment).

In some embodiments, the fluorescent response is intense due to a sharp phase transition that occurs between the hydrophobicity-driven micellar self-assembly (non-fluorescent OFF state) and the cooperative dissociation of these micelles (fluorescent ON state) at predefined low pH.

In some embodiments, the micelles described herein have a pH transition point and an emission spectrum. In some embodiments, the pH transition point is between 4-8 or between 6-7.5. In other embodiments, the pH transition point is between 4.8-5.5. In certain embodiments, the pH transition point is about 4.8, 4.9, 5.0, 5.1, 5.2, 5.3, 5.4, or 5.5. In some embodiments, the emission spectrum is between 700-900 nm. In some embodiments, the emission spectra is between 750-850 nm.

In some instances, the pH-sensitive micelle compositions described herein have a narrow pH transition range. In some embodiments, the micelles described herein have a pH transition range (ΔpH_{10-90%}) of less than 1 pH unit. In various embodiments, the micelles have a pH transition range of less than about 0.9, less than about 0.8, less than about 0.7, less than about 0.6, less than about 0.5, less than about 0.4, less than about 0.3, less than about 0.2, less than about 0.1 pH unit. In some embodiments, the micelles have a pH transition range of less than about 0.5 pH unit. In some embodiments, the pH transition range is less than 0.25 pH units. In some embodiments, the pH transition range is less than 0.15 pH units.

In some embodiments, the pH-sensitive composition has a fluorescence activation ratio. In some embodiments, the fluorescence activation ratio is greater than 25. In some embodiments, the fluorescence activation ratio is greater than 50.

In some embodiments, when the intracellular environment is imaged, the cell is contacted with the micelle under conditions suitable to cause uptake of the micelle. In some embodiments, the intracellular environment is part of a cell. In some embodiments, the part of the cell is lysosome or an endosome. In some embodiments, the extracellular environment is of a tumor or vascular cell. In some embodiments, the extracellular environment is intravascular or extravascular. In some embodiments, imaging the pH of an intracellular or extracellular environment comprises imaging a metastatic disease. In some embodiments, the metastatic disease is a cancer. In some embodiments, the tumor is from a solid cancer. In some embodiments, the tumor is from a non-solid cancer. In some embodiments, imaging the pH of the tumor environment comprises imaging the lymph node or nodes. In some embodiments, imaging the pH of the tumor environment allows determination of the tumor size or tumor margins during surgery.

In another aspect, is a method of imaging the pH of an intercellular or extracellular environment, the method comprising:

(a) contacting the intracellular or extracellular environment with the block copolymer or the pharmaceutical composition disclosed herein; and

(b) detecting one or more optical signals from the intracellular or extracellular environment, wherein a detected optical signal indicates that the micelle comprising the one or more block copolymers of Formula (II) has reached its pH transition point and disassociated.

In some embodiments, the optical signal is a fluorescent signal.

In some embodiments, the extracellular environment is a tumor or vascular cell. In some embodiments, the extracellular environment is intravascular or extravascular.

In some embodiments, the pH of an intracellular or extracellular environment comprises imaging the pH of a tumor environment. In some embodiments, imaging the pH of the tumor environment comprises imaging the lymph node or nodes. The sentinel lymph node is the first lymph node or group of nodes draining a cancer and are the first organs to be reached by metastasizing cancer cells from the tumor. In some embodiments, imaging the pH of the lymph node or nodes informs the surgical resection of the lymph node. In some embodiments, imaging the pH of the lymph node or nodes informs the staging of the cancer metastasis. In some embodiments, imagining the pH of lymph node or nodes enables patient management.

In some embodiments, imaging the pH of the tumor environment allows for determination of the tumor size or tumor margins. In some embodiments, imaging the pH of the tumor environment allows for tumor staging. In some embodiments, imaging of the pH of the tumor environment allows for management of patient outcomes. In some embodiments, imaging the pH of the tumor environment allows for more precise removal of the tumor during surgery. In some embodiments, imaging the pH of the tumor environment enables the detection of a residual metastatic disease. In some embodiments, imaging the pH of the tumor environment informs the determination of satellite, multi-focal, or occult tumors.

In some embodiments, imaging the pH of the tumor environment informs the detection of occult disease.

In some embodiments, the pharmaceutical composition is administered to a patient in need thereof prior to imaging a tumor. In some embodiments, the pharmaceutical composition is administered to a patient in need thereof prior to imaging a tumor for staging prior to surgery.

In some embodiments, the pharmaceutical composition is administered to a patient in need thereof before surgery. In some embodiments, the pharmaceutical composition is administered to a patient in need thereof after a surgery. In some embodiments, surgery is a tumor resection.

In another aspect, is a method of resecting a tumor in a patient in need thereof, the method comprising:

(a) detecting one or more optical signals from the tumor or a sample thereof from the patient administered with an effective dose of a block copolymer or pharmaceutical composition disclosed herein, wherein a detected optical signal(s) indicate the presence of the tumor; and

(b) resecting the tumor via a surgery.

In some embodiments, optical signals indicate the margins of the tumor.

In some embodiments, the optical signal is a fluorescent signal.

In some embodiments, the tumor is at least 90% resected.

In some embodiments, the tumor is at least 95% resected.

In some embodiments, the tumor is at least 99% resected.

In some embodiments, the tumor is resected along with clean margins. In some embodiments, the clean margins are non-fluorescing tissues. In some embodiments, the non-fluorescing tissues are non-cancerous tissues. In some embodiments, the lack of fluorescence in the wound bed after the removal of the tumor or lymph node(s) after resection indicates removal of the tumor.

In some embodiments, the tumor is a solid tumor. In some embodiments, the tumor is a pan tumor. In some embodiments, the solid tumor is from a cancer. In some embodiments, the cancer is breast cancer, head and neck squamous cell carcinoma (NHSCC), lung cancer, ovarian cancer, prostate cancer, bladder cancer, urethral cancer, esophageal cancer, colorectal cancer, brain cancer, or skin cancer (including melanoma and sarcoma). In some embodiments, the cancer is breast cancer, head and neck squamous cell carcinoma (NHSCC), esophageal cancer, or colorectal cancer. In some embodiments, the cancer is breast cancer. In some embodiments, the cancer is head and neck squamous cell carcinoma (NHSCC). In some embodiments, the cancer is ovarian cancer. In some embodiments, the cancer is prostate cancer. In some embodiments, the cancer is esophageal cancer. In some embodiments, the cancer is colorectal cancer. In some embodiments, the cancer is brain cancer. In some embodiments, the cancer is skin cancer treatable by Mohs surgery.

In another aspect, is a method of treating cancer, the method comprising:

(a) detecting one or more optical signals in a cancer patient in need thereof administered with an effective dose of a block copolymer or a pharmaceutical composition disclosed herein, wherein a detected optical signal indicates the presence of a cancerous tumor; and

(b) removing the cancerous tumor, thereby treating the cancer.

In some embodiments, the method further comprising imaging body cavity of the cancer patient, or imaging the cancerous tumor or a slice or specimen thereof (e.g., fresh or formalin fixed), optionally by back-table fluorescence-guided imaging after the removal from the patient. In some embodiments, the method of treating cancer further comprises imaging the cancerous tumor after the removal to ensure clean borders. In some embodiments, a clean border is indicated by the lack of tumor in the wound bed. In some embodiments, a clean border is indicated when no fluorescence is detected in the sample or in the wound bed. In some embodiments, the clean borders indicate that the entire cancerous tumor has been removed. In some embodiments, the clean borders indicate all cancerous have been removed.

In another aspect, is a method of minimizing recurrence of cancer for at least five years, the method comprising:

(a) detecting one or more optical signals in a cancer patient in need thereof administered with an effective dose of a block copolymer or a pharmaceutical composition disclosed herein, wherein a detected optical signal indicates the presence of the cancerous tumor; and

(b) treating the cancer to minimize the recurrence if the one or more optical signals is detected.

In another aspect, is a method of detecting a cancerous tumor, the method comprising:

(a) detecting one or more optical signals in a cancer patient in need thereof administered with an effective dose of a block copolymer or a pharmaceutical composition disclosed herein, wherein the presence of the tumor indicates the recurrence of the cancer; and

(b) treating the recurrence of the cancer.

In some embodiments, the tumor is from a cancer. In some embodiments, the cancer is breast cancer, head and neck squamous cell carcinoma (NHSCC), lung cancer, ovarian cancer, prostate cancer, bladder cancer, urethral cancer, esophageal cancer, colorectal cancer, brain, skin (including melanoma and sarcoma). In some embodiments, the cancer is breast cancer, head and neck squamous cell carcinoma (NHSCC), esophageal cancer, or colorectal cancer. In some embodiments, the cancer is ovarian cancer. In some embodiments, the cancer is prostate cancer.

In some embodiments, the method further comprises imaging the tumor with an intra-operative camera or an endoscopic camera. In some embodiments, the intra-operative camera is a near-infrared (NIR) camera. In some embodiments of the methods disclosed herein, the intra-operative camera or an endoscopic camera, is a camera compatible with indocyanine green.

### Dosing

In some embodiments, the pharmaceutical composition is administered to a patient in need thereof. In some embodiments, the patient in need thereof is a mammal. In some embodiments, the patient in need thereof is a human. Ins some embodiments, the mammal is not a human. In some embodiments, the mammal is a canine, feline, bovine, pig, rabbit, or equine. In some embodiments, the mammal is a canine or feline. In some embodiments, the mammal is a cat. In some embodiments, the mammal is a horse. In some embodiments, the mammal is a cow. In some embodiments, the mammal is a pig. In some embodiments, the mammal is a rabbit. In some embodiments, the mammal is a canine.

The block copolymer of Formula (II) or a hydrate, solvate, tautomer, or pharmaceutically acceptable salt thereof of the present disclosure may be in the form of compositions suitable for administration to a subject. In general, such compositions are “pharmaceutical compositions” comprising a block copolymer of Formula (II) or a hydrate, solvate, tautomer, or pharmaceutically acceptable salt thereof and one or more pharmaceutically acceptable or physiologically acceptable diluents, carriers or excipients. In certain embodiments, the block copolymer of Formula (II) or a hydrate, solvate, tautomer, or pharmaceutically acceptable salt thereof are present in a therapeutically acceptable amount. The pharmaceutical compositions may be used in the methods of the present invention; thus, for example, the pharmaceutical compositions can be administered ex vivo or in vivo to a subject in order to practice the therapeutic and prophylactic methods and uses described herein.

In some embodiments, the pharmaceutical composition is administered from about 1 to 2 weeks prior to a surgery. In some embodiments, the pharmaceutical composition is administered about 2 weeks prior to surgery. In some embodiments, the pharmaceutical composition is administered about 1 week prior to surgery. In some embodiments, the pharmaceutical composition is administered from about 16 hours to about 80 hours prior to a surgery. In some embodiments, the pharmaceutical composition is administered from about 24 hours to about 32 hours prior to a surgery. In some embodiments, the pharmaceutical composition is administered from about 16 hours to about 32 hours prior to a surgery. In some embodiments, the pharmaceutical composition is administered from about 1 hour to about 5 hours prior to surgery. In some embodiments, the pharmaceutical composition is administered from about 3 hours to about 9 hours prior to surgery.

In some embodiments, pharmaceutical composition is administered at least 1 hour, at least 2 hours, at least 3 hours, at least 4 hours, at least 5 hours, at least 6 hours, at least 7 hours, at least 8 hours, at least 10 hours, at least 12 hours, at least 14 hours, at least 16 hours, at least 18 hours, at least 20 hours, at least 24 hours, at least 28 hours, at least 32 hours, at least 80 hours, at least 1 day, at least 2 days, at least 3 days, at least 4 days, at least 5 days, at least 6 days, at least 7 days, at least 1 week, or at least 2 weeks prior to surgery.

In some embodiments, the pharmaceutical composition is administered from about 1 to 2 weeks prior to imaging the tumor. In some embodiments, the pharmaceutical composition is administered about 2 weeks prior to imaging the tumor. In some embodiments, the pharmaceutical composition is administered about 1 week prior to imaging the tumor. In some embodiments, the pharmaceutical composition is administered from about 16 hours to about 80 hours prior to imaging the tumor. In some embodiments, the pharmaceutical composition is administered from about 24 hours to about 32 hours prior to imaging the tumor. In some embodiments, the pharmaceutical composition is administered from about 16 hours to about 32 hours prior to imaging the tumor. In some embodiments, the pharmaceutical composition is administered from about 3 hours to about 9 hours prior to imaging the tumor. In some embodiments, the pharmaceutical composition is administered from about 1 hour to about 5 hours prior to imaging the tumor. In some embodiments, the pharmaceutical composition is administered from about 1 hour to about 32 hours, about 2 hours to about 32 hours, 16 hours to about 32 hours, or about 20 hours to about 28 hours prior to an imaging the tumor.

In some embodiments, pharmaceutical composition is administered at least 1 hour, at least 2 hours, at least 3 hours, at least 4 hours, at least 5 hours, at least 6 hours, at least 7 hours, at least 8 hours, at least 10 hours, at least 12 hours, at least 14 hours, at least 16 hours, at least 18 hours, at least 20 hours, at least 24 hours, at least 28 hours, at least 32 hours, at least 80 hours, at least 1 day, at least 2 days, at least 3 days, at least 4 days, at least 5 days, at least 6 days, at least 7 days, at least 1 week, or at least 2 weeks prior to imaging the tumor.

In some embodiments, the block copolymer of Formula (II) or a hydrate, solvate, tautomer, or pharmaceutically acceptable salt thereof pharmaceutical compositions described herein are provided at the maximum tolerated dose (MTD) for the block copolymer of Formula (II). In other embodiments, the amount of the block copolymer of Formula (II) or a hydrate, solvate, tautomer, or pharmaceutically acceptable salt thereof pharmaceutical composition administered is from about 10% to about 90% of the maximum tolerated dose (MTD), from about 25% to about 75% of the MTD, or about 50% of the MTD. In some other embodiments, the amount of the block copolymer of Formula (II) or a hydrate, solvate, tautomer, or pharmaceutically acceptable salt thereof pharmaceutical compositions administered is from about 5%, 10%, 15%, 20%, 25%, 30%, 35%, 40%, 45%, 50%, 55%, 60%, 65%, 70%, 75%, 80%, 85%, 90%, 95%, 99%, or higher, or any range derivable therein, of the MTD for the block copolymer of Formula (II).

### Definitions

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

“Pharmaceutically acceptable,” as used herein, refers a material, such as a carrier or diluent, which does not abrogate the biological activity or properties of the block copolymer, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a block copolymer of Formula (II) with an acid. In some embodiments, the block copolymer of Formula (A) (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (-L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (-L); salicylic acid; sebacic acid; stearic acid; succinic acid; sulfuric acid; tartaric acid (+L); thiocyanic acid; toluenesulfonic acid (p); and undecylenic acid.

In some embodiments, a block copolymer of Formula (II) is prepared as a chloride salt, sulfate salt, bromide salt, mesylate salt, maleate salt, citrate salt or phosphate salt.

In some embodiments, pharmaceutically acceptable salts are obtained by reacting a block copolymer of Formula (II) with a base. In some embodiments, the block copolymer of Formula (II) is acidic and is reacted with a base. In such situations, an acidic proton of the block copolymer of Formula (II) is replaced by a metal ion, e.g., lithium, sodium, potassium, magnesium, calcium, or an aluminum ion. In some cases, block copolymers described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, block copolymers described herein form salts with amino acids such as, but not limited to, arginine, lysine, and the like. Acceptable inorganic bases used to form salts with block copolymers that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the block copolymers provided herein are prepared as a sodium salt, calcium salt, potassium salt, magnesium salt, melamine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of block copolymers having the structure of Formula (II), as well as active metabolites of these compounds having the same type of activity.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

As used herein, “pH responsive system,” “pH responsive composition,” “micelle,” “pH-responsive micelle,” “pH-sensitive micelle,” “pH-activatable micelle” and “pH-activatable micellar (pHAM) nanoparticle” are used interchangeably herein to indicate a micelle comprising one or more compounds, which disassociates depending on the pH (e.g., above or below a certain pH). As a non-limiting example, at a certain pH, the block copolymers of Formula (II) is substantially in micellar form. As the pH changes (e.g., decreases), the micelles begin to disassociate, and as the pH further changes (e.g., further decreases), the block copolymers of Formula (II) is present substantially in disassociated (non-micellar) form.

As used herein, “pH transition range” indicates the pH range over which the micelles disassociate.

As used herein, “pH transition value” (pH) indicates the pH at which half of the micelles are disassociated.

A “nanoprobe” is used herein to indicate a pH-sensitive micelle which comprises an imaging labeling moiety. In some embodiments, the labeling moiety is a fluorescent dye. In some embodiments, the fluorescent dye is indocyanine green dye.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular, intratumoral, or infusion), topical and rectal administration. Those of skill in the art are familiar with administration techniques that can be employed with the compounds and methods described herein. In some embodiments, the compounds and compositions described herein are administered orally. In some embodiments, the compositions described herein are administered intravenously.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the standard deviation of error for the device or method being employed to determine the value, for example about ±10% of a stated number or a 10% below the lower limit and 10% above the upper limit for values listed for a stated range. Following longstanding patent law, the words “a” and “an,” for example when used in conjunction with the word “comprising” in the claims or specification, denotes one or more, unless specifically noted.

### EXAMPLES

### Example 1. Synthesis of Block Copolymers

Block copolymers of Formula (II) described herein are synthesized using standard synthetic techniques or using methods known in the art.

Unless otherwise indicated, conventional methods of mass spectroscopy, NMR, HPLC, protein chemistry, biochemistry, recombinant DNA techniques and pharmacology are employed. Block copolymers are prepared using standard organic chemistry techniques such as those described in, for example, March's Advanced Organic Chemistry, 6^{th} Edition, John Wiley and Sons, Inc.

Some abbreviations used herein are as follows:
DCIS Ductal carcinoma in situ
DCM: dichloromethane
DMAP: 4-dimethylaminopyridine
DMF: dimethyl formamide
DMF-DMA: N,N-dimethylformamide dimethyl acetal
EDCI: 1-ethyl-3-(3-dimethylaminopropyl) carbodiimide
EtOAc: ethyl acetate
EtOH: ethanol
ICG-OSu: indocyanine green succinamide ester
MeOH: methanol
PMDETA: N,N,N′,N″,N″-Pentamethyldiethylenetriamine
TEA: triethyl amine
AUC Area under the curve
AUCₐₗₗ AUC from time=0 to the last time point (including conc=0)
AUCₗₐₛₜ AUC from time=0 to the last time point with a reportable concentration
AUC₀₋₂₄ₕᵣ AUC from time 0 to 24 hr
BC Breast Cancer
BLS Bread loaf slide
BQL Below the limit of quantitation
C₁₀ₘ Plasma concentration at 10 min
Cₘₐₓ Maximum plasma concentration
CNR Contrast to noise ratio
CRC Colorectal cancer
EC Esophageal cancer
FFPE or FF Formalin-fixed paraffin-embedded or formalin fixed
GMP Good manufacturing practice
GLP Good laboratory practice
GPC Gel permeation chromatography
HNSCC Head and neck squamous cell carcinoma
Hr Hour(s)
ISR Incurred sample reanalysis
IV Intravenous
kg Kilogram
LLOQ Lower limit of quantitation of assay
MFI Mean fluorescent intensity
mg Milligram(s)
mL Milliliters(s)
μg Microgram(s)
NC Not calculated
NR Not reported
OC Ovarian cancer
PK Pharmacokinetics
PPV Positive predictive value
PrC Prostate cancer
ROI Region of interest
r²adj Coefficient of determination adjusted for sample size
SEC Size exclusion chromatography
SOC Standard of care
SOP Standard Operating Procedure
TBR Tumor to background ratio
T_{1/2} Half-life
Tₘₐₓ Time of maximum concentration.

Block copolymers of Formula (II) were synthesized using a 5-step process. Steps 1 thru 4 were performed in a controlled manufacturing environment. Intermediate 8 (polydibutyl amine, PDBA) was synthesized by atom transfer radical polymerization (ATRP, Step 4) of 3 (PEG-Br, a macroinitiator), 7 ((dibutylamino)ethyl methacrylate, DBA-MA), and 4 (aminoethylmethylacrylate hydrochloride, AMA-MA). The final step included preparation of the Compound 1 by covalently attaching 8 (the diblock copolymer backbone of PDBA) to 9 (the ICG fluorophore (ICG-OSu)). In step 5, all raw materials, solvents and reagents used are either National Formulary (NF) or United States Pharmacopeia (USP) verified except for intermediate 9 (ICG-OSu) which was sourced as a GMP manufactured material. As a precautionary measure Compound 1 was stored at −80° C.±15° C. and protected from light.

Schemes 1 and 2, provides a process flow chart followed by a detailed description of the manufacturing process.

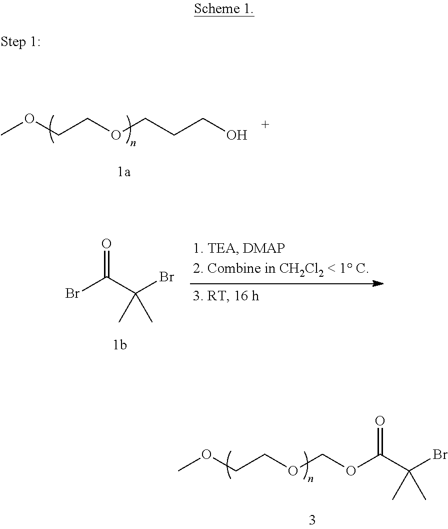

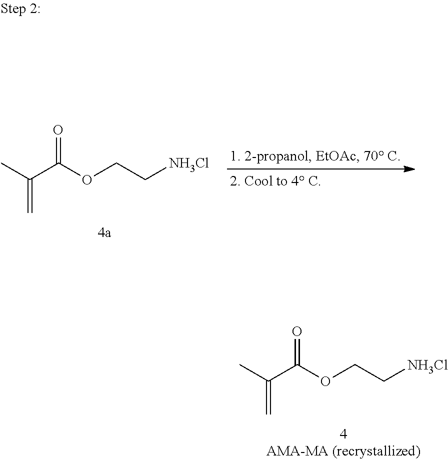

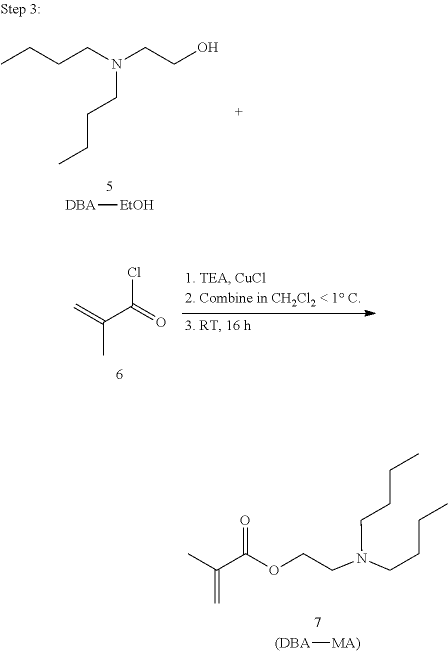

### Step 1:

Synthesis: Poly(ethyleneglycol) methyl ether (PEG-OH) 1a, trimethylamine, 4-(dimethylamino) pyridine (DMAP) in dichloromethane (CH₂Cl₂) were cooled in an ice bath. α-Bromoisobutyryl bromide 1b in dichloromethane was then added dropwise to the flask while the flask was maintained in the ice bath. The reaction mixture was allowed to warm to room temperature (RT) and stirred for 16 hrs.

Purification: The reaction mixture was then added slowly to a beaker containing ˜10-fold excess by volume of diethyl ether under stirring to precipitate the crude product 3. The crude product was then filtered and dried in a vacuum oven. The dried, crude 3 was recrystallized from ethanol five times and dried in a vacuum oven to yield the purified 3 (PEG-Br macroinitiator). A typical yield is 40%-70% with a purity of >93% (High-performance liquid chromatography [HPLC] area %).

### Step 2:

Recrystallization: Crude 2-Aminoethylmethacrylate hydrochloride (AMA-MA monomer), 2-propanol 4a and ethyl acetate were combined and heated to 70° C. until the solid was dissolved. The solution was filtered through a pre-heated Buchner funnel containing celite. The filtered solution was allowed to cool to RT and then further cooled to 2-8° C. to crystallize over a period of 8 to 16 hr. The resulting crystalline solids were allowed to warm to RT and were then filtered and washed 3 times with cold ethyl acetate. The isolated crystalline product was dried under vacuum to give purified 4 and stored at −80° C. for use in Step 4. A typical yield is 40%-70% with purity indicated by solubility in a use-test and also a sharp melting point (<3° C.) in the range of 102-124° C.

### Step 3:

Synthesis: 2-(Dibutylamino) ethanol (DBA-EtOH, 5), trimethylamine, copper (I) chloride (CuCl) and dichloromethane were combined in a flask and cooled in an ice bath. Methacryloyl chloride 6 was then added dropwise to the flask while maintaining in the ice bath. The reaction mixture was allowed to warm to RT and was stirred for 16 hrs. The reaction mixture was then cooled in an ice bath and filtered. The filtrate was transferred to a separatory funnel and the organic phase was washed twice with saturated aqueous solution of sodium bicarbonate (NaHCO₃) followed by one wash with DI water. The organic phase was then dried over anhydrous sodium sulfate (Na₂SO₄), filtered and the solvents were removed in vacuo using a rotary evaporator to yield the monomer product 7 as a liquid.

Purification: Additional CuCl was added as a stabilizer and the product was purified by vacuum distillation. The clear to yellowish distillate 7 (DBA-MA) was transferred to an amber vial and stored at −80° C. for use in Step 4. A typical yield is 30%-60% with a purity of >93% (HPLC area %).

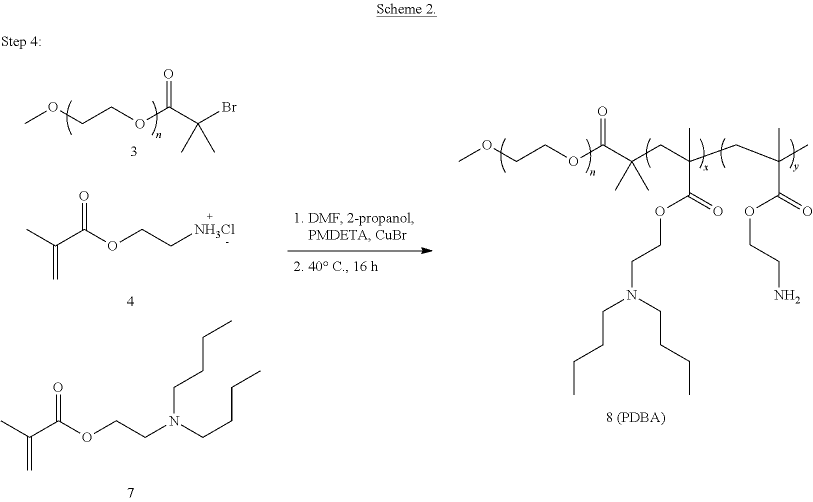

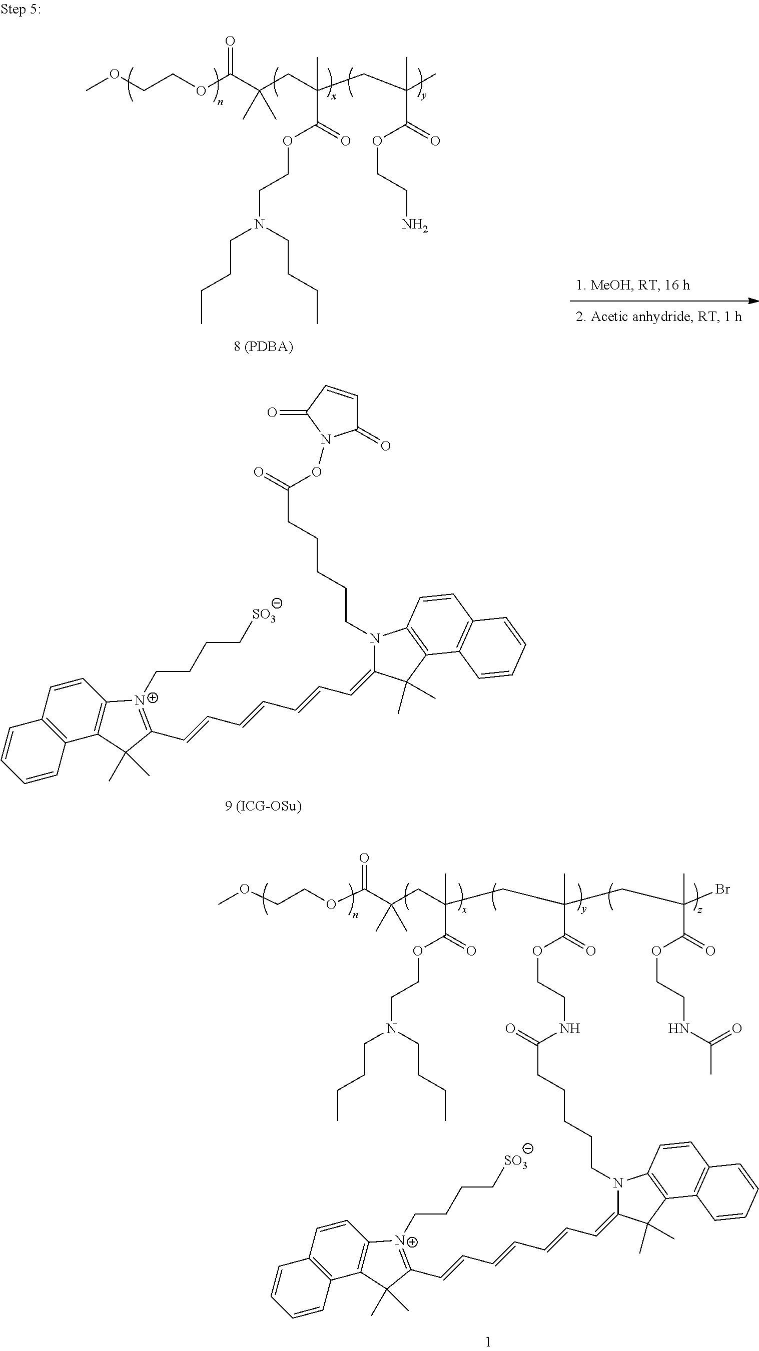

### Step 4:

Synthesis: Intermediate 3 was added to a flask and dissolved in a mixture of the dimethylformamide (DMF) and 2-propanol by gently heating the flask. The contents of the flask were allowed to cool to room temperature and 4 and 7 (AMA-MA monomer and DBA-MA monomer, respectively) were added to the solution followed by N,N,N′,N″,N″-Pentamethyldiethylenetriamine (PMDETA). The reaction mixture was stirred and then subjected to four freeze-pump-thaw cycles under nitrogen to remove air (oxygen). The reaction mixture was treated with copper (I) bromide (CuBr) while still frozen and was subjected to three cycles of vacuum and flushing with nitrogen to ensure that entrapped air was removed and the reaction was then allowed to warm to 40° C. in an oil bath. The reaction mixture was allowed to further react for 16 hr. At the completion of the reaction, the mixture was diluted with tetrahydrofuran and filtered through a bed of the aluminum oxide (Al₂O₃). The solvents were removed from the filtrate using rotary evaporation and dried under vacuum.

Purification: The dried crude product was dissolved with methanol and purified by tangential flow filtration through a 10k MWCO Pellicon® 2 Mini Filter cassette with methanol. The solvent was then removed using rotary evaporation. The purified intermediate 8 (PEO₁₁₃-b-(DBA₈₀₋₁₅₀-r-AMA₁₋₃), PDBA) was dried under vacuum and stored at −80° C. for use in Step 5. A typical yield is 60%-90% with a purity of >93% (HPLC area %). In some cases, the product is a mixture of conjugated and unconjugated polymer.

### Step 5:

Synthesis: Intermediate 8 (PDBA) was dissolved in methanol (MeOH) with the help of a sonication bath. The methanol solution was then added to 9 (ICG-OSu). The reaction was stirred at room temperature for 16 h while protected from light. At the end of the reaction, a 6-fold excess acetic anhydride was added to the reaction mixture and allowed to mix for 1-1.5 h to produce the crude product Compound 1.

Purification: The crude product purified by tangential flow filtration through a 10k Pellicon® 2 Mini Ultrafiltration Module with methanol. The solvent of the filtered solution was removed in vacuo to produce Compound 1 which was protected from light and stored at −80° C. A typical yield is >70% with a purity of NLT 95% (SEC).

Analysis: Analysis of relative molar mass distribution is conducted via a custom gel permeation chromatography (GPC) method with refractive index (RI) detection and two Agilent PLgel Mixed-D 300×7.5 mm columns. Sample chromatograms are compared to a calibration curve constructed from polystyrene standards from 580 to 1,074,000 g/mol to calculate molar mass distribution.

### Example 2. Storage of Compound 1

The current presentation of Compound 1 for injection is a 3 mg/mL green aqueous solution stored at −80° C. Vials are thawed to room temperature prior to intravenous administration 15 mg/min for Phase 1 and 30 mg/min for Phase 2.

### Example 3. Stability of Compound 1

Stability data indicate that Compound 1, 3.0 mg/mL injection is stable at the long-term storage condition of −80° C. for up to 24 months, the duration thus far. No significant changes were observed in the assay or the level of related substances and impurities or any of the other attributes tested at the storage condition. Updated stability results are provided in Table 1 and Table 2.

TABLE 1
Stability results for Compound 1 at −80° C. from 0 to 12 months

| Storage Condition: −80° C. | | Time (months) | | | |
|---|---|---|---|---|---|
| Test | Criteria^{a} | 0 | 1 | 6 | 12 |
| Appearance | Greenish | Pass | Pass | Pass | Pass |
| | waxy solid | | | | |
| Identification - | ±2% of | Pass | Pass | Pass | Pass |
| SEC (RT) | the RT of | | | | |
| | the RS | | | | |
| Assay^{RID} - | NMT | 96.4% | 98.5% | 102.3% | 90.4% |
| Polymeric | 90.0% | | | | |
| Content | | | | | |
| SEC (% wt) | | | | | |
| Content^{784 nm} | | 80.9% | 82.0% | 86.2% | 74.7% |
| (% wt) | | | | | |
| % ICG^{784 nm} | | 1.3% | 1.4% | 1.4% | 1.3% |
| (% wt) Impuri- | | | | | |
| ties | | | | | |
| Specified: ICG- | Report | 1.9% | 1.8% | 1.7% | 1.7% |
| like | levels | | | | (RRT = |
| | and RRT | | | | 1.49-1.71) |
| Unspecified | Report | ND | ND | ND | ND |
| | all > | | | | |
| | LOQ and | | | | |
| | RRT | | | | |
| | NMT | | | | |
| Total | 7.0% | 1.9% | 1.8% | 1.7% | 1.7% |
| Impurities | | | | | |
| Water Content | | 0.48% | 0.33% | 0.54% | 0.74% |
| Microbial | | | | | |
| Limits^{b} | | | | | |
| Total aerobic | NMT | <1 cfu/mg | — | — | <1 cfu/mg |
| count | 10³ | | | | |
| | cfu/g | | | | |
| Total yeast and | NMT | <1 cfu/mg | | | <1 cfu/mg |
| mold | 10² | | | | |
| | cfu/g | | | | |

TABLE 2
Stability results for Compound 1 at −80°
C. continued from 18 to 24 months

| Storage Condition: −80° C. | | Time (months) | |
|---|---|---|---|
| Test | Criteriaa | 18 | 24 |
| Appearance | Greenish waxy solid | Pass | Pass |
| Identification -SEC (RT) | ±2% of the RT of the | Pass | Pass |
| | RS | | |
| Assay^{RID} - Polymeric | NMT 90.0% | 100.4% | 99.7% |
| Content SEC (% wt) | | | |
| Content^{784 nm} (% wt) | | 94.7% | 98.1% |
| % ICG^{784 nm} (% wt) | | 1.5% | 1.5% |
| Impurities | | | |
| Specified: ICG-like | Report levels and RRT | 0.6% | 0.6% |
| | | (RRT = | (RRT = |
| | | 1.45-1.70) | 1.40-1.60) |
| | | (n = 2) | (n = 2) |
| Unspecified | Report all > LOQ and | ND | ND |
| | RRT NMT 7.0% | | |
| Total Impurities | | 0.6% | 0.6% |
| Water Content | | 0.64% | 0.51% |
| Microbial Limits^{b} | | | |
| Total aerobic count | NMT 10³ cfu/g | — | <1 cfu/mg |
| Total yeast and mold | NMT 10² cfu/g | | <1 cfu/mg |

^{a}Acceptace criteria submitted in IND 139686; results evaluated against GMP stability criteria in place at time of lot manufacture
^{b}Test performed annually for stability
LOQ = Limit of quantitation (0.3%);
NLT = Not less than;
NMT = Not more than;
ND = Not detected;
RRT = Relative retention time;
SEC = Size exclusion chromatography;
Wt = Weight

### Example 4. PK Effects in Humans

### Phase 1 Study Objectives

Phase 1 is a single-Principal Investigator, non-randomized, open-label, single-arm, cross-sectional study to evaluate the safety, PK profile, and imaging feasibility of Compound 1 in patients with solid cancers who require surgical excision. The main purpose of this study was to investigate the safety, PK, and feasibility of Compound 1 as an intra-operative optical imaging agent to detect tumors and metastatic lymph nodes in solid cancers. The study was intended to investigate the optimal dose range of Compound 1 for an adequate TBR and CNR of fluorescence obtained intraoperatively at 24 (±8) hours post dosing and with ex vivo specimens using ICG compatible cameras and imaging devices.

Phase 1 enrolled 30 patients with solid cancers (HNSCC, breast cancer, esophageal cancer, or colorectal cancer) who have a biopsy-confirmed diagnosis of respective tumor types and are scheduled to undergo surgical resection of the tumor. The study design included a standard 3+3 design for the dose-escalation portion (Phase 1a; N=18 maximum) followed by a dose-expansion portion (Phase 1b; N=15). All patients received a single I.V. dose of Compound 1, followed by routine surgery approximately 24 hours after infusion of Compound 1.

Phase 1a was a dose-finding study performed in 5 cohorts of 3 patients each. The dose levels evaluated were 0.3, 0.5, 0.8, 0.1, and 1.2 mg/kg, in this sequence. Inter-cohort dose escalation took place after the last patient in the previous cohort completed the Day 10 safety assessment. Safety, PK, and imaging feasibility were evaluated in both the Phase 1a and 1b portions of the study. Patient safety is assessed during the study and for up to 10 days post-dose.

During surgery, intraoperative images of Compound 1 fluorescence were obtained from the primary tumors and metastatic lymph nodes as well as the surrounding tissue which include normal noncancerous tissues using NIR camera(s). This may be in vivo and/or ex vivo imaging of resected specimens. If the surgeon considered it safe, up to a maximum of 10 study related biopsies were taken from the regions with Compound 1 fluorescence that were otherwise not suspected as tumor clinically. Feasibility to image tumors with Compound 1 using multiple NIR cameras was evaluated.

Tumor specimens were processed for histology according to the standard pathology practice used in clinical cancer care. Diagnosis on margins, selected histological features necessary for clinical decision making were provided. Fluorescence images were collected from the tumor and lymph node specimens and study-related biopsies. Margin width and number of positive margins were noted and correlated to the location of fluorescence in the margins. From this, the correlation between Compound 1 fluorescence and histopathology were calculated.

### Disposition and Demographics

All patients received a single dose of Compound 1 and completed the study. All patients were included in the imaging, PK, and safety analyses.

Thirty (30) patients with 4 different tumor types (HNSCC, n=13; BC, n=11 patients; CRC, n=3; EC, n=3) undergoing routine surgery received a single dose of ONM-100 at 24 (±8) hours before their planned surgery (Table 3).

In Phase 1a, a total of 3 male and 12 female patients between 34 and 80 years of age and with a body mass index between 17.4 and 37.1 kg/m2 participated in the study. All patients were white (Caucasian) and none of the patients were of Hispanic or Latino ethnicity. A total of 8 patients had a diagnosis of HNSCC and 7 patients had BC.

In Phase 1b, a total of 5 male and 10 female patients between 45 and 85 years of age and with a body mass index between 18.9 and 39.4 kg/m2 participated in the study. All patients were white (Caucasian) and none of the patients were of Hispanic or Latino ethnicity. A total of 5 patients had a diagnosis of HNSCC, 4 patients had BC, 3 patients had CRC, and 3 patients had EC. The mean age in Phase 1b (68 years) was higher than in Phase 1a (58 years).

TABLE 3
Patient demographic and baseline characteristics

| | | | | | | |
|---|---|---|---|---|---|---|
| Demographic | Cohort 1 | Cohort 2 | Cohort 3 | Cohort 4 | Cohort 5 | All |
| or Baseline | 0.3 mg/kg | 0.5 mg/kg | 0.8 mg/kg | 0.1 mg/kg | 1.2 mg/kg | Cohorts |
| Parameter | (N = 3) | (N = 3) | (N = 3) | (N = 3) | (N = 3) | (N = 15) |

Cancer Type, n

| | | | | | | |
|---|---|---|---|---|---|---|
| Breast | 3 | 1 | 2 | 0 | 1 | 7 |
| HNSCC | 0 | 2 | 1 | 3 | 2 | 8 |

Age, years

| | | | | | | |
|---|---|---|---|---|---|---|
| Mean | 53.3 | 56.7 | 61.3 | 68.3 | 50.7 | 58.1 |
| Range | 35-63 | 50-69 | 53-78 | 46-80 | 34-70 | 34-80 |

Gender, n

| | | | | | | |
|---|---|---|---|---|---|---|
| Male | 0 | 1 | 0 | 1 | 1 | 3 |
| Female | 3 | 2 | 3 | 2 | 2 | 12 |

Ethnicity, n

| | | | | | | |
|---|---|---|---|---|---|---|
| Hispanic | 0 | 0 | 0 | 0 | 0 | 0 |
| or Latino | | | | | | |
| Non-Hispanic | 3 | 3 | 3 | 3 | 3 | 15 |
| or Latino | | | | | | |

Race, n

| | | | | | | |
|---|---|---|---|---|---|---|
| White | 3 | 3 | 3 | 3 | 3 | 15 |

Body mass index, kg/m²

| | | | | | | |
|---|---|---|---|---|---|---|
| Mean | 30.3 | 27.8 | 31.1 | 22.7 | 22.4 | 26.9 |

### Pharmacokinetic Results

Study design: Single Compound 1 IV dose was administered as a 1-5 minute IV infusion to patients in five cohorts (0.1, 0.3, 0.5, 0.8, and 1.2 mg/kg) with three patients per cohort in Phase 1a and 15 patients at a dose of 1.2 mg/kg in Phase 1b. Patient demographic information including tumor types is presented in Table 4 and 5 for Phase 1a and Table 5 for Phase 1b. Intertek Pharmaceutical Services (San Diego, Calif.) determined Compound 1 plasma concentrations using a validated direct fluorescence reader assay. Pacific BioDevelopment (Davis, Calif.) performed the PK analysis.

Sample collection: Blood samples were collected prior to infusion and at 10 minutes and 0.5, 1, 3, 8, 24, 48, 72, and 240 hours after infusion.

TABLE 4
Phase 1a Patient dosing, demographic, and disposition information

| | Dose | | Cancer | | Age | | Completed |
|---|---|---|---|---|---|---|---|
| Cohort | Received | Patient | Type | Sex | (years) | Race | Study |
| B | 0.3 mg/kg | ON1101 | BC | F | 62 | White | yes |
| | | ON1102 | BC | F | 35 | White | yes |
| | | ON1103 | BC | F | 63 | White | yes |
| C | 0.5 mg/kg | ON1104 | HNSCC | F | 50 | White | yes |
| | | ON1105 | BC | F | 51 | White | yes |
| | | ON1106 | HNSCC | M | 69 | White | yes |
| D | 0.8 mg/kg | ON1107 | BC | F | 78 | White | yes |
| | | ON1108 | HNSCC | F | 53 | White | yes |
| | | ON1109 | BC | F | 53 | White | yes |
| A | 0.1 mg/kg | ON1110 | HNSCC | M | 46 | White | yes |
| | | ON1111 | HNSCC | F | 80 | White | yes |
| | | ON1112 | HNSCC | F | 79 | White | yes |
| E | 1.2 mg/kg | ON1113 | HNSCC | M | 70 | White | yes |
| | | ON1114 | HNSCC | F | 48 | White | yes |
| | | ON1115 | BC | F | 34 | White | yes |

F = female;
M = male

TABLE 5
Phase 1a Pharmacokinetic parameters estimated by noncompartmental analysis

| Dose | | Cₘₐₓ | Tₘₐₓ | AUCₐₗₗ | AUCₗₐₛₜ | T_{1/2} | AUC_{0-24 hr} | C_{10 m} |
|---|---|---|---|---|---|---|---|---|
| mg/kg | Subj | (μg/mL) | (hr) | (hr*μg/mL) | (hr*μg/mL) | (hr) | (hr*μg/mL) | (μg/mL) |
| 0.1 | ON1110 | 0.00 | 0.170 | 0.00 | NC | NC | 0.00 | 0.00 |
| | ON1111 | 0.00 | 0.170 | 0.00 | NC | NC | 0.00 | 0.00 |
| | ON1112 | 0.00 | 0.170 | 0.00 | NC | NC | 0.00 | 0.00 |
| | N | 3 | 3 | 3 | 0 | 0 | 3 | 3 |
| | Mean | 0.00 | 0.170 | 0.00 | NC | NC | 0.00 | 0.00 |
| | SD | 0.00 | 0.00 | 0.00 | NC | NC | 0.00 | 0.00 |
| | Median | 0.00 | 0.170 | 0.00 | NC | NC | 0.00 | 0.00 |
| 0.3 | ON1101 | 12.5 | 8.00 | 437 | 292 | NC | 292 | 10.7 |
| | ON1102 | 10.4 | 0.170 | 4.68 | 3.43 | NC | 4.68 | 10.4 |
| | ON1103 | 16.5 | 3.00 | 2040¹ | 902 | NC | 296 | 14.8 |
| | N | 3 | 3 | 3 | 3 | 0 | 3 | 3 |
| | Mean | 13.1 | 3.72 | 826 | 399 | NC | 197 | 12.0 |
| | SD | 3.10 | 3.96 | 1070 | 459 | NC | 167 | 2.46 |
| | Median | 12.5 | 3.00 | 437 | 292 | NC | 292 | 10.7 |
| 0.5 | ON1104 | 22.7 | 3.00 | 314 | 160 | NC | 314 | 22.0 |
| | ON1105 | 17.1 | 3.00 | 1980 | 969 | NR² | 340 | 16.2 |
| | ON1106 | 14.0 | 0.500 | 213 | 101 | NC | 213 | 13.6 |
| | N | 3 | 3 | 3 | 3 | 0 | 3 | 3 |
| | Mean | 17.9 | 2.17 | 835 | 410 | NC | 289 | 17.3 |
| | SD | 4.41 | 1.44 | 991 | 485 | NC | 67.5 | 4.30 |
| | Median | 17.1 | 3.00 | 314 | 160 | NC | 314 | 16.2 |
| 0.8 | ON1107 | 23.0 | 1.00 | 905 | 756 | 74.5 | 416 | 21.7 |
| | ON1108 | 19.6 | 0.170 | 518 | 354 | NR² | 354 | 19.6 |
| | ON1109 | 18.2 | 0.170 | 1790 | 927 | 83.4 | 380 | 18.2 |
| | N | 3 | 3 | 3 | 3 | 2 | 3 | 3 |
| | Mean | 20.3 | 0.447 | 1070 | 679 | 79.0 | 383 | 19.8 |
| | SD | 2.47 | 0.479 | 653 | 294 | 6.33 | 31.1 | 1.76 |
| | Median | 19.6 | 0.170 | 905 | 756 | 79.0 | 380 | 19.6 |
| 1.2 | ON1113 | 28.0 | 0.170 | 348 | 184 | 32.5 | 348 | 28.0 |
| | ON1114 | 34.1 | 0.170 | 1160 | 970 | 46.5 | 563 | 34.1 |
| | ON1115 | 33.0 | 0.170 | 1020 | 891 | 30.6 | 574 | 33.0 |
| | N | 3 | 3 | 3 | 3 | 3 | 3 | 3 |
| | Mean | 31.7 | 0.170 | 842 | 682 | 36.5 | 495 | 31.7 |
| | SD | 3.25 | 0.00 | 433 | 433 | 8.68 | 128 | 3.25 |
| | Median | 33.0 | 0.170 | 1020 | 891 | 32.5 | 563 | 33.0 |

¹AUC may be overestimated due to > LLOQ concentration at 72 hours observed after two BQL values at 24 and 48 hours.
²Not Reportable, R² < 0.8.

Analysis: Plasma concentration versus time profiles were generated for each patient. Pharmacokinetic parameters were estimated using Phoenix WinNonlin (version 8.0). According to SOP, concentrations reported as BQL were set to 0 except for the 0.5 h sample for subject #ON1102, which was set to LLOQ/2 (5 ag/mL value used in parameter calculations) and the 24 and 48 h samples.

The parameters estimated were Cₘₐₓ, Tₘₐₓ, T_{1/2}, AUCₗₐₛₜ, AUCₐₗₗ and AUC₀₋₂₄ₕᵣ. If there were less than three data points in the terminal phase of the curve, the program did not calculate a T_{1/2} (NC). If the coefficient of determination for the terminal slope estimation was less than 0.8, T_{1/2} was not reported (NR). AUC extrapolated to infinity is not reported for any data set because in all cases the % extrapolated AUC was greater than 20% and thus the AUC_{inf} estimate would not be reliable. The concentration at 10 minutes, the first time point measured (C10m), was also reported for each patient.

The area under the plasma concentration versus time curves from dosing to the last time point with a measurable concentration (AUCₗₐₛₜ) was estimated by the linear trapezoid method. The last three or more time points were used to estimate the elimination rate constant (λz) which was used to estimate the terminal-phase half-life (T_{1/2}) and AUC from zero to infinity (AUC_{INF}) from the following equations:

T_{1/2}=ln(2)/λz

AUC_{INF}=AUC₀₋ₜ+Cₜ/λz

where Cₜ is the last measurable concentration.

### Phase 1a

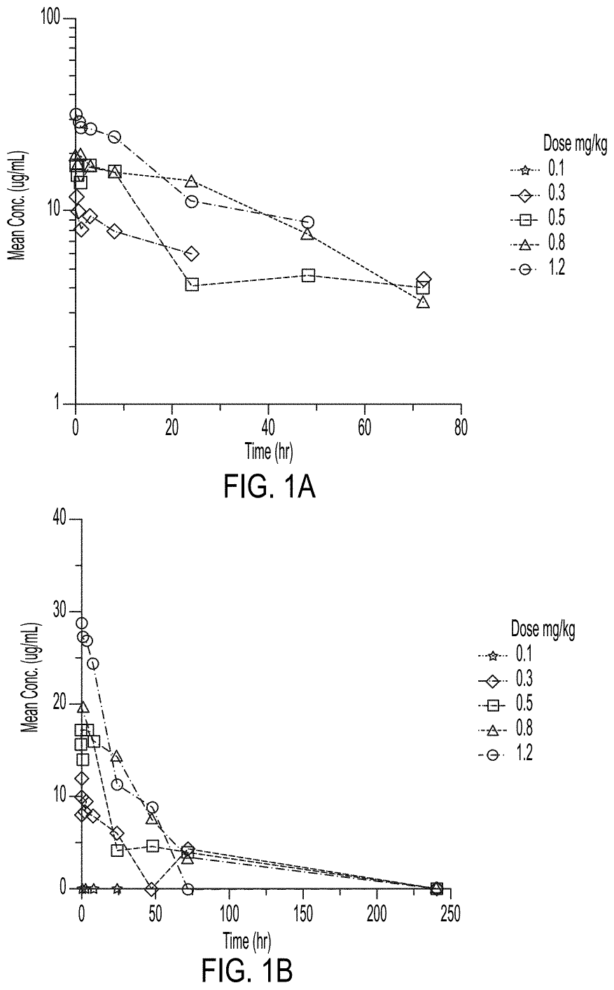

Patient demographic data for Phase 1a are presented in Table 4. Individual plasma concentrations are shown in Table 6. Individual pharmacokinetic parameter estimates, and group summary statistics are presented in Table 6. Plots of mean plasma concentrations (log and linear) versus time are presented in FIGS. 1A-1B.

Compound 1 was not measurable in any subject samples following a dose of 0.1 mg/kg.

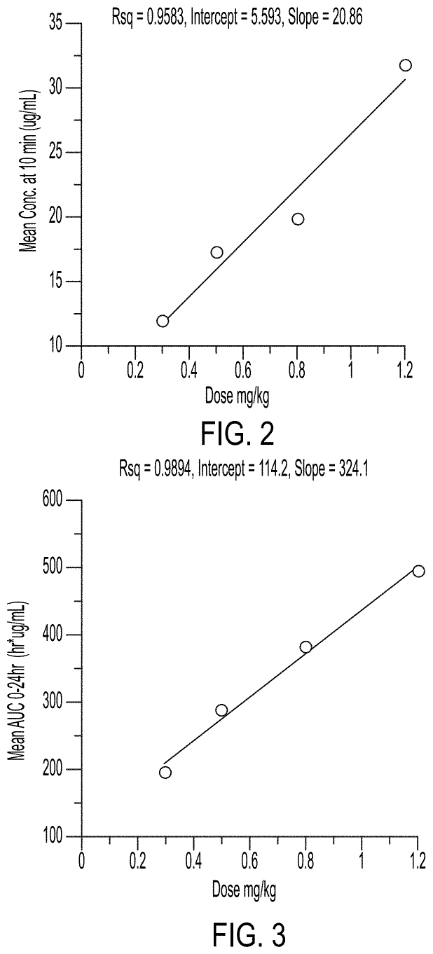

Exposure was dose-related. Cₘₐₓ, AUCₗₐₛₜ, AUCₐₗₗ and AUC₀₋₂₄ₕᵣ were higher with higher doses. The concentration at 10 minutes after dosing and the AUC₀₋₂₄ₕᵣ are plotted versus dose in FIG. 2 and FIG. 3, respectively. The plots show the results of performing linear regression on the parameter versus dose data. Data from the 0.1 mg/kg dose group in which all plasma values were reported as BQL are excluded from these plots. The study was not powered to perform a statistical analysis for dose proportionality; however, the linear regression indicates a strong correlation between exposure and dose.

Mean C₁₀ values were 12.0, 17.3, 19.8 and 31.7 pg/mL at the 0.3, 0.5, 0.8, and 1.2 mg/kg doses, respectively. Mean AUC₀₋₂₄ₕ were 197, 289, 383, and 495 μg-h/mL. Mean terminal-phase half-life values were only quantifiable from the 0.8 and 1.2 mg/kg dose groups and were 79.0 and 36.5 h, respectively.

TABLE 6
Phase 1a individual subject plasma concentrations

| | | Sample time (hr) | | | | | | | | | |
|---|---|---|---|---|---|---|---|---|---|---|---|
| | | Predose | 0.170 | 0.500 | 1.00 | 3.00 | 8.00 | 24.0 | 48.0 | 72.0 | 240 |
| Dose | | | | | | | | | | | |
| mg/kg | Subject | Plasma Conc (μg/mL) ^{a} | | | | | | | | | |
| 0.100 | ON1110 | BQL | BQL | BQL | BQL | BQL | BQL | BQL | NS | NS | BQL |
| | ON1111 | BQL | BQL | BQL | BQL | BQL | BQL | BQL | BQL | BQL | BQL |
| | ON1112 | BQL | BQL | BQL | BQL | BQL | BQL | BQL | BQL | BQL | BQL |
| | N | 3 | 3 | 3 | 3 | 3 | 3 | 3 | 2 | 2 | 3 |
| | Mean | 0.00 | 0.00 | 0.00 | 0.00 | 0.00 | 0.00 | 0.00 | 0.00 | 0.00 | 0.00 |
| | SD | 0.00 | 0.00 | 0.00 | 0.00 | 0.00 | 0.00 | 0.00 | 0.00 | 0.00 | 0.00 |
| 0.300 | ON1101 | BQL | 10.7 | NS | 11.4 | 12.0 | 12.5 | 12.1 | BQL | BQL | BQL |
| | ON1102 | BQL | 10.4 | 5.00^{b} | BQL | BQL | BQL | BQL | BQL | BQL | BQL |
| | ON1103 | BQL | 14.8 | 15.1 | 12.9 | 16.5 | 11.2 | BQL | BQL | 13.5 | BQL |
| | N | 3 | 3 | 2 | 3 | 3 | 3 | 2 | 2 | 3 | 3 |
| | Mean | 0.00 | 12.0 | 10.1 | 8.10 | 9.50 | 7.90 | 6.05 | 0.00 | 4.50 | 0.00 |
| | SD | 0.00 | 2.46 | 7.14 | 7.05 | 8.53 | 6.87 | 8.56 | 0.00 | 7.79 | 0.00 |
| 0.500 | ON1104 | BQL | 22.0 | 16.7 | 15.8 | 22.7 | 19.3 | BQL | BQL | BQL | BQL |
| | ON1105 | BQL | 16.2 | 15.9 | 13.8 | 17.1 | 14.6 | 12.4 | 14.0 | 12.0 | BQL |
| | ON1106 | BQL | 13.6 | 14.0 | 12.6 | 11.9 | 13.9 | BQL | BQL | BQL | BQL |
| | N | 3 | 3 | 3 | 3 | 3 | 3 | 3 | 3 | 3 | 3 |
| | Mean | 0.00 | 17.3 | 15.5 | 14.1 | 17.2 | 15.9 | 4.13 | 4.67 | 4.00 | 0.00 |
| | SD | 0.00 | 4.30 | 1.39 | 1.62 | 5.40 | 2.94 | 7.16 | 8.08 | 6.93 | 0.00 |
| 0.800 | ON1107 | BQL | 21.7 | 19.3 | 23.0 | 20.0 | 16.7 | 16.0 | 12.4 | BQL | NS |
| | ON1108 | BQL | 19.6 | 17.6 | 18.2 | 14.4 | 15.1 | 13.7 | BQL | BQL | BQL |
| | ON1109 | BQL | 18.2 | 17.1 | 18.0 | 17.6 | 16.6 | 13.7 | 10.8 | 10.3 | BQL |
| | N | 3 | 3 | 3 | 3 | 3 | 3 | 3 | 3 | 3 | 2 |
| | Mean | 0.00 | 19.8 | 18.0 | 19.7 | 17.3 | 16.1 | 14.5 | 7.73 | 3.43 | 0.00 |
| | SD | 0.00 | 1.76 | 1.15 | 2.83 | 2.81 | 0.896 | 1.33 | 6.74 | 5.95 | 0.00 |
| 1.20 | ON1113 | BQL | 28.0 | 23.9 | 23.9 | 24.5 | 20.5 | BQL | BQL | BQL | BQL |
| | ON1114 | BQL | 34.1 | 30.5 | 32.1 | 28.4 | 24.4 | 18.3 | 15.6 | BQL | BQL |
| | ON1115 | BQL | 33.0 | 31.8 | 25.8 | 27.8 | 28.4 | 15.6 | 10.8 | BQL | BQL |
| | N | 3 | 3 | 3 | 3 | 3 | 3 | 3 | 3 | 3 | 3 |
| | Mean | 0.00 | 31.7 | 28.7 | 27.3 | 26.9 | 24.4 | 11.3 | 8.80 | 0.00 | 0.00 |
| | SD | 0.00 | 3.25 | 4.24 | 4.29 | 2.10 | 3.95 | 9.88 | 7.99 | 0.00 | 0.00 |

^{a} BQL (<10 μg/mL), set = 0 for PK analysis
NS = No sample
Note:
LLOQ = 10 μg/mL

### Phase 1b

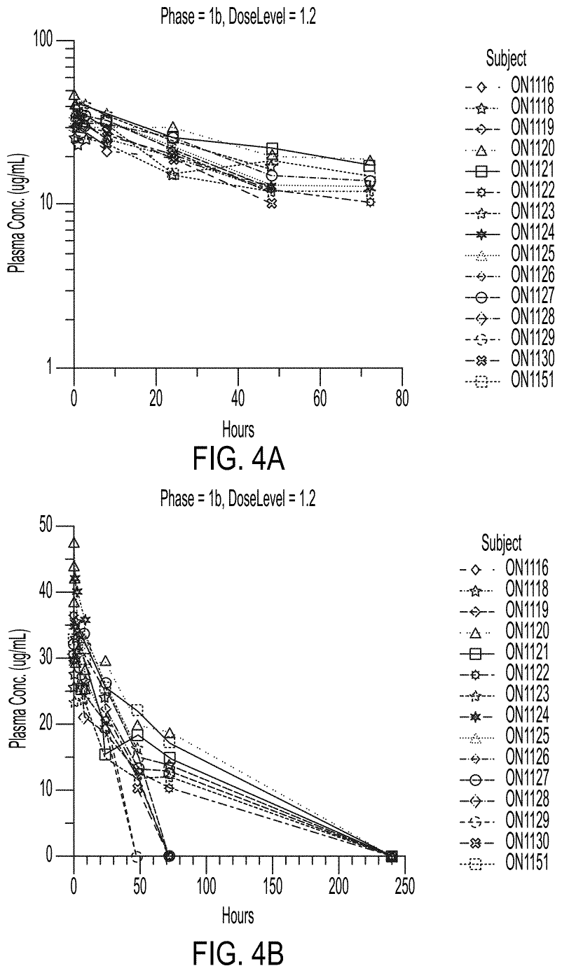

Patient demographic data for Phase 1b are presented in Table 7. Individual plasma concentrations for the patients are shown in Table 8. Individual pharmacokinetic parameter estimates, and group summary statistics are presented in Table 9. Plots of individual plasma concentrations (log and linear) versus time are presented in FIG. 4A-4B.

Mean C₁₀ₘ was 33.2 μg/mL and mean AUC₀₋₂₄ₕᵣ was 638 ag-hr/mL. Mean terminal-phase half-life was 46.4 h.

TABLE 7
Phase 1b patient demographic, and disposition
dosed at 1.2 mg/kg of Compound 1

| | | | Age | |
|---|---|---|---|---|
| Patient | Cancer Type | Sex | (years) | Race |
| ON1116 | BC | F | 45 | White |
| ON1118 | HNSCC | F | 69 | White |
| ON1119 | HNSCC | M | 85 | White |
| ON1120 | CRC | M | 69 | White |
| ON1121 | HNSCC | M | 84 | White |
| ON1122 | EC | M | 69 | White |
| ON1123 | BC | F | 60 | White |
| ON1124 | EC | F | 75 | White |
| ON1125 | HNSCC | F | 58 | White |
| ON1126 | EC | M | 47 | White |
| ON1127 | HNSCC | F | 77 | White |
| ON1128 | BC | F | 76 | White |
| ON1129 | CRC | F | 75 | White |
| ON1130 | CRC | F | 75 | White |
| ON1151 | BC | F | 60 | White |

TABLE 8
Phase 1b patient plasma concentrations

| | | Sample time (hr) | | | | | | | | | |
|---|---|---|---|---|---|---|---|---|---|---|---|
| | | Predose | 0.170 | 0.500 | 1.00 | 3.00 | 8.00 | 24.0 | 48.0 | 72.0 | 240 |
| Dose | | | | | | | | | | | |
| mg/kg | Subject | Plasma Conc (μg/mL) ^{a} | | | | | | | | | |
| 1.2 | ON1116 | BQL | 28.0 | 32.0 | 26.8 | 26.4 | 23.6 | 21.0 | 12.8 | BQL | BQL |
| | ON1118 | BQL | 28.5 | 29.8 | 29.9 | 25.4 | 24.5 | 20.3 | 12.3 | BQL | BQL |
| | ON1119 | BQL | 27.5 | 29.2 | 25.9 | 27.5 | 21.1 | 18.9 | BQL | BQL | BQL |
| | ON1120 | BQL | 38.6 | 35.2 | 33.0 | 34.3 | 28.3 | 29.8 | 19.9 | 18.7 | BQL |
| | ON1121 | BQL | 30.3 | 30.3 | 31.3 | 32.4 | 31.3 | 15.5 | 18.5 | 14.9 | BQL |
| | ON1122 | BQL | 31.4 | 28.5 | 32.6 | 31.3 | 26.1 | 20.2 | 12.6 | 10.4 | BQL |
| | ON1123 | BQL | 25.5 | 27.6 | 23.3 | 25.0 | 23.6 | 15.0 | 12.0 | 12.0 | BQL |
| | ON1124 | BQL | 40.0 | 40.0 | 42.2 | 40.1 | 35.8 | 23.9 | 16.3 | BQL | BQL |
| | ON1125 | BQL | 47.6 | 44.1 | 42.2 | 35.3 | 32.6 | 22.7 | 13.3 | 12.8 | BQL |
| | ON1126 | BQL | 32.6 | 32.0 | 31.3 | 30.3 | 25.0 | 19.5 | 11.8 | BQL | BQL |
| | ON1127 | BQL | 31.3 | 28.9 | 29.9 | 34.3 | 33.6 | 26.2 | 15.0 | 13.8 | BQL |
| | ON1128 | BQL | 36.5 | 34.5 | 37.7 | 29.6 | 27.9 | 20.3 | 12.5 | BQL | BQL |
| | ON1129 | BQL | 32.1 | 32.9 | 34.1 | 31.5 | 32.4 | 21.2 | BQL | BQL | BQL |
| | ON1130 | BQL | 35.8 | 36.4 | 35.4 | 33.3 | 27.8 | 18.6 | 10.2 | BQL | NS |
| | ON1151 | BQL | 33.0 | 31.4 | 34.3 | 31.2 | 30.0 | 25.6 | 22.0 | 17.3 | BQL |
| | N | 15 | 15 | 15 | 15 | 15 | 15 | 15 | 15 | 15 | 14 |
| | Mean | 0.00 | 33.2 | 32.9 | 32.7 | 31.2 | 28.2 | 21.2 | 12.6 | 6.66 | 0.00 |
| | SD | 0.00 | 5.72 | 4.58 | 5.39 | 4.08 | 4.29 | 3.93 | 6.10 | 7.62 | 0.00 |
| | Median | 0.00 | 32.1 | 32.0 | 32.6 | 31.3 | 27.9 | 20.3 | 12.6 | 0.00 | 0.00 |

^{a} BQL (<10 μg/mL), set = 0 for PK analysis
NS = No sample
Note:
LLOQ = 10 μg/mL

TABLE 9
Phase 1b: Pharmacokinetic parameters estimated by noncompartmental analysis

| | Cₘₐₓ | Tₘₐₓ | AUCₐₗₗ | AUCₗₐₛₜ | T_{1/2} | AUC_{0-24 hr} | C_{10 m} |
|---|---|---|---|---|---|---|---|
| Subject | (μg/mL) | (hr) | (hr*μg/mL) | (hr*μg/mL) | (hr) | (hr*μg/mL) | (μg/mL) |
| ON1116 | 32.0 | 0.500 | 1120 | 968 | 45.5 | 562 | 28.0 |
| ON1118 | 29.9 | 1.00 | 1100 | 957 | 42.6 | 565 | 28.5 |
| ON1119 | 29.2 | 0.500 | 747 | 520 | NR¹ | 520 | 27.5 |
| ON1120 | 38.6 | 0.170 | 3350 | 1780 | 77.9 | 721 | 38.6 |
| ON1121 | 32.4 | 3.00 | 2690 | 1430 | NR | 625 | 30.3 |
| ON1122 | 32.6 | 1.00 | 2150 | 1280 | 43.3 | 606 | 31.4 |
| ON1123 | 27.6 | 0.500 | 2120 | 1110 | 61.4 | 502 | 25.5 |
| ON1124 | 42.2 | 1.00 | 1460 | 1270 | 34.4 | 787 | 40.0 |
| ON1125 | 47.6 | 0.170 | 2550 | 1480 | 38.4 | 730 | 47.6 |
| ON1126 | 32.6 | 0.170 | 1100 | 961 | 33.6 | 585 | 32.6 |
| ON1127 | 34.3 | 3.00 | 2740 | 1580 | 46.8 | 740 | 31.3 |
| ON1128 | 37.7 | 1.00 | 1170 | 1020 | 34.5 | 630 | 36.5 |
| ON1129 | 34.1 | 1.00 | 939 | 684 | 33.4 | 684 | 32.1 |
| ON1130 | 36.4 | 0.500 | 1090 | 971 | 27.7 | 626 | 35.8 |
| ON1151 | 34.3 | 1.00 | 3190 | 1740 | 83.0 | 693 | 33.0 |
| N | 15 | 15 | 15 | 15 | 13 | 15 | 15 |
| Mean | 34.8 | 0.967 | 1840 | 1180 | 46.4 | 638 | 33.2 |
| SD | 5.18 | 0.888 | 889 | 368 | 17.3 | 84.8 | 5.72 |
| Median | 34.1 | 1.00 | 1460 | 1110 | 42.6 | 626 | 32.1 |

¹Not Reportable, R² < 0.8

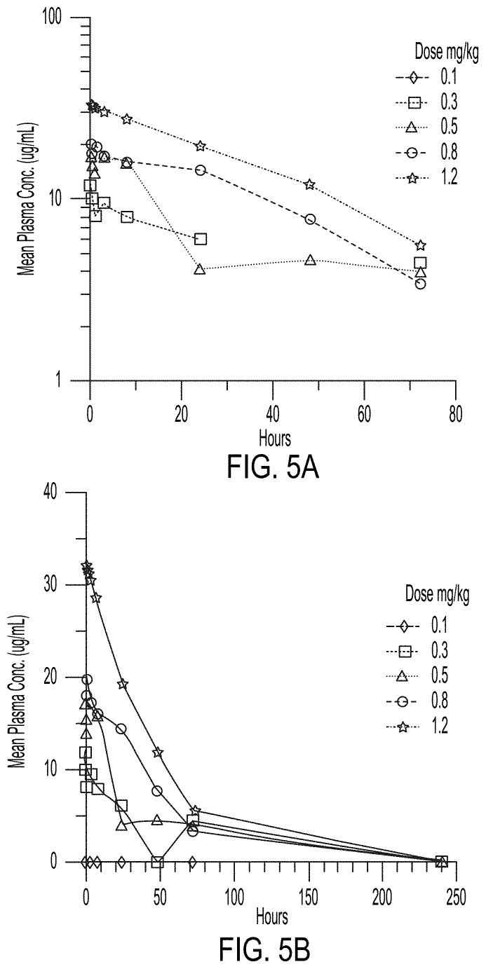

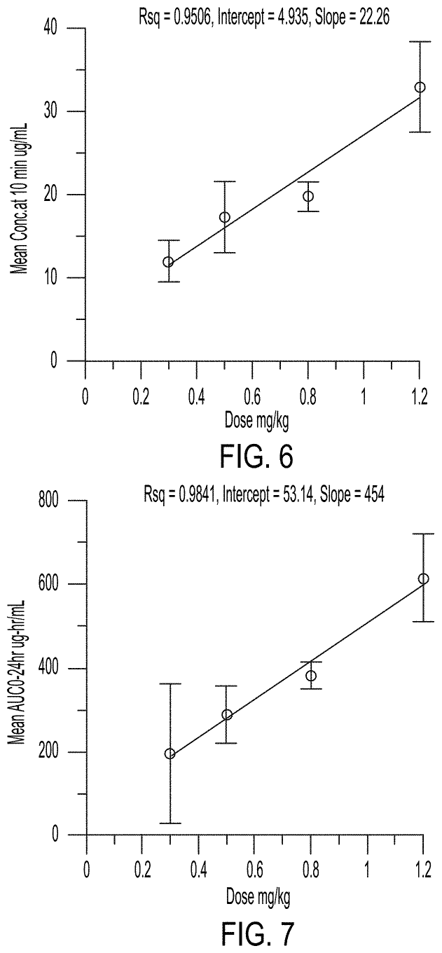

Mean plasma concentrations are plotted versus time by dose group for all patients in Phase 1a and Phase 1b combined are presented in FIG. 5A (log plot) and FIG. 5B (linear plot). Plots of mean concentration at 10 minutes (C₁₀ₘ) and AUC₀₋₂₄ₕᵣ versus dose for all patients in Phase 1a and Phase 1b are plotted in FIG. 6 and FIG. 7. The data support the observation made based on Phase 1a data that exposure is dose proportional.

Individual patient pharmacokinetic parameters and summary statistics organized by tumor type for all patients from Phase 1a and Phase 1b treated with 1.2 mg/kg are presented in Table 10. There were no apparent differences among the estimated pharmacokinetic parameters based on tumor type. C₁₀ₘ values ranged from 31.2 to 35.5 μg/mL and AUC₀₋₂₄ₕᵣ values range from 585 to 677 μg-hr/mL.

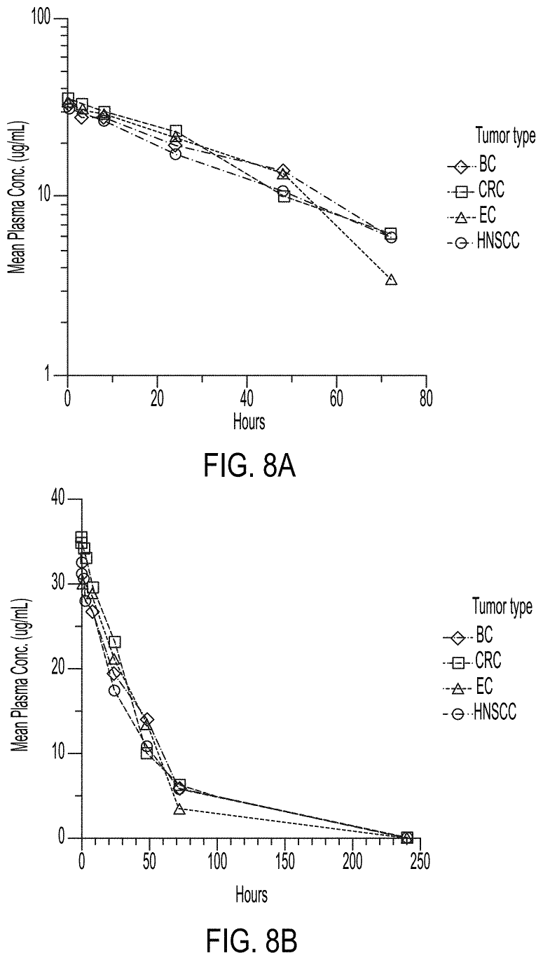

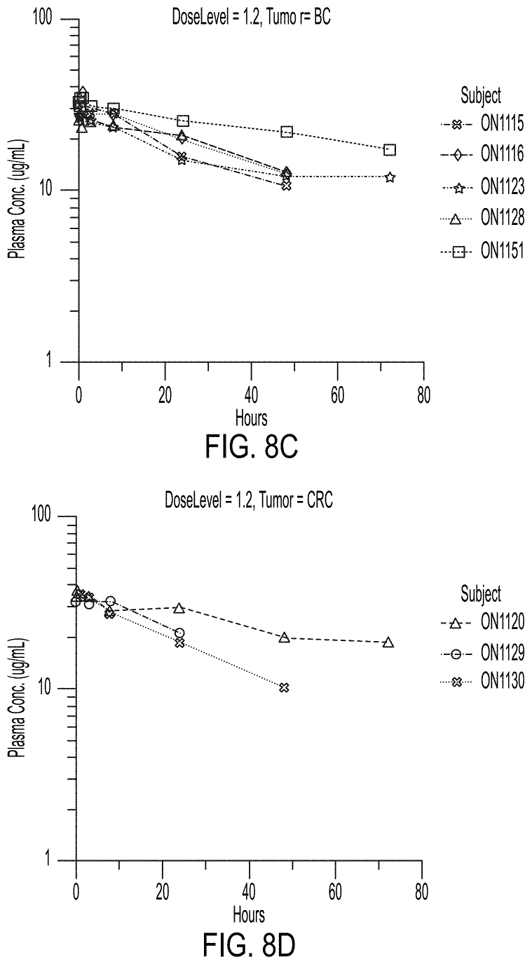

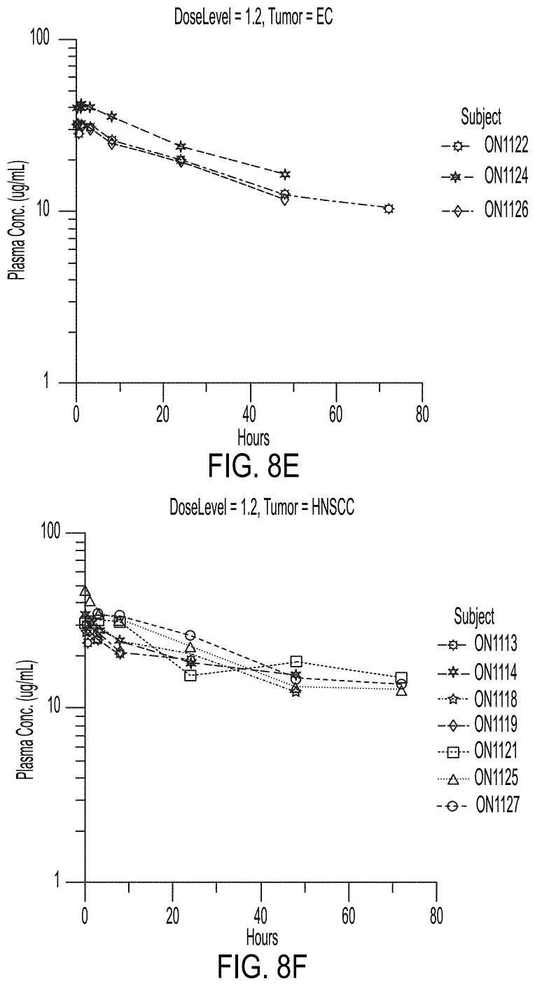

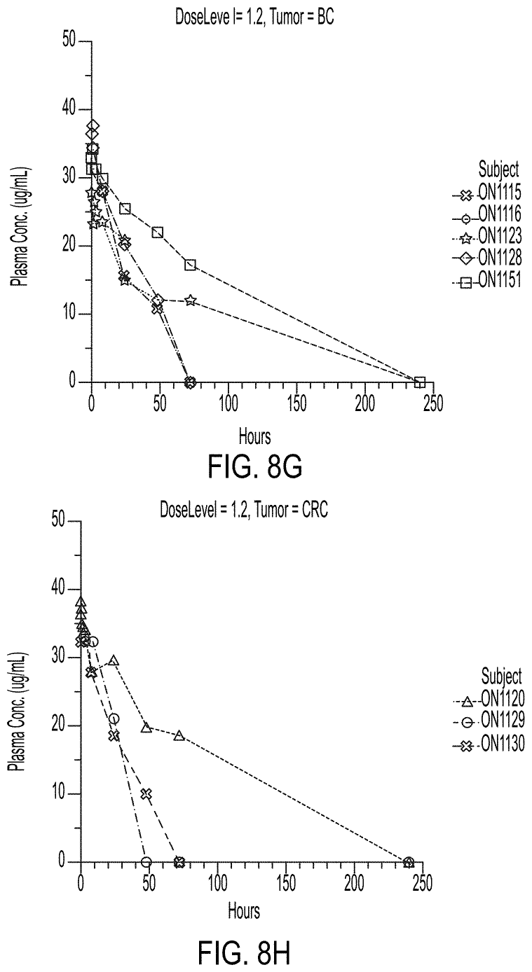

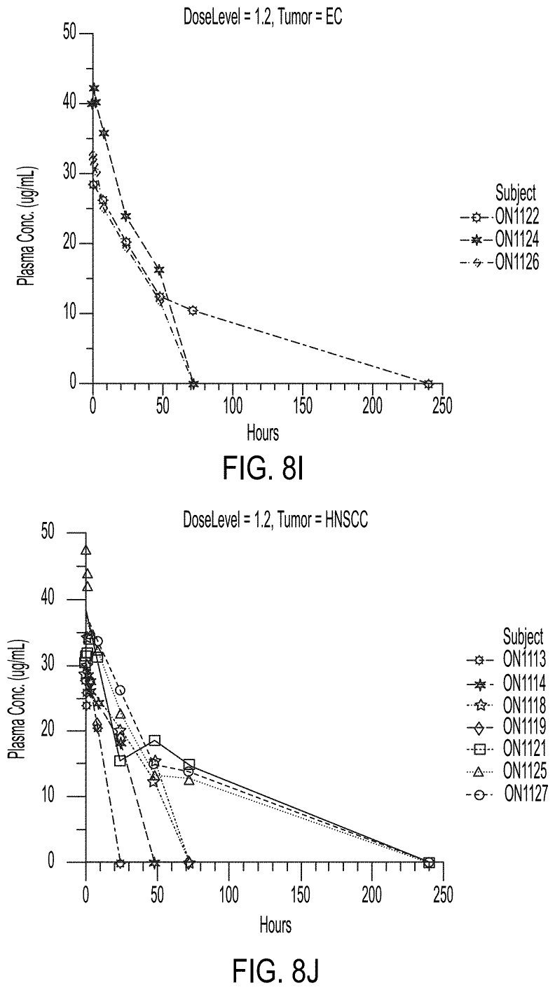

Plots of mean plasma concentrations versus time for each tumor type are shown in FIG. 8A (log plot) and FIG. 8B (linear plot). These plots illustrate that there are no apparent differences in Compound 1 pharmacokinetics among tumor types tested. Individual plasma concentration versus time plots for each type of tumor are presented in FIGS. 8C-8F (log plots) and FIGS. 8G-8J (linear plots).

TABLE 10
Pharmacokinetic parameters estimated by noncompartmental analysis patients
receiving 1.2 mg/kg (Phase 1a and Phase 1b) sorted by cancer type

| Cancer | | Cₘₐₓ | Tₘₐₓ | AUCₐₗₗ | AUCₗₐₛₜ | T_{1/2} | AUC_{0-24 hr} | C_{10 m} |
|---|---|---|---|---|---|---|---|---|
| Type | Subj | (μg/mL) | (hr) | (hr*μg/mL) | (hr*μg/mL) | (hr) | (hr*μg/mL) | (μg/mL) |
| BC | ON1115 | 33.0 | 0.170 | 1020 | 891 | 30.6 | 574 | 33.0 |
| | ON1116 | 32.0 | 0.500 | 1120 | 968 | 45.5 | 562 | 28.0 |
| | ON1123 | 27.6 | 0.500 | 2120 | 1110 | 61.4 | 502 | 25.5 |
| | ON1128 | 37.7 | 1.00 | 1170 | 1020 | 34.5 | 630 | 36.5 |
| | ON1151 | 34.3 | 1.00 | 3190 | 1740 | 83.0 | 693 | 33.0 |
| | N | 5 | 5 | 5 | 5 | 5 | 5 | 5 |
| | Mean | 32.9 | 0.634 | 1730 | 1150 | 51.0 | 592 | 31.2 |
| | SD | 3.67 | 0.360 | 931 | 340 | 21.5 | 72.3 | 4.40 |
| | Median | 33.0 | 0.500 | 1170 | 1020 | 45.5 | 574 | 33.0 |
| CRC | ON1120 | 38.6 | 0.170 | 3350 | 1780 | 77.9 | 721 | 38.6 |
| | ON1129 | 34.1 | 1.00 | 939 | 684 | 33.4 | 684 | 32.1 |
| | ON1130 | 36.4 | 0.500 | 1090 | 971 | 27.7 | 626 | 35.8 |
| | N | 3 | 3 | 3 | 3 | 3 | 3 | 3 |
| | Mean | 36.4 | 0.557 | 1790 | 1150 | 46.3 | 677 | 35.5 |
| | SD | 2.25 | 0.418 | 1350 | 569 | 27.5 | 48.2 | 3.26 |
| | Median | 36.4 | 0.500 | 1090 | 971 | 33.4 | 684 | 35.8 |
| EC | ON1122 | 32.6 | 1.00 | 2150 | 1280 | 43.3 | 606 | 31.4 |
| | ON1124 | 42.2 | 1.00 | 1460 | 1270 | 34.4 | 787 | 40.0 |
| | ON1126 | 32.6 | 0.170 | 1100 | 961 | 33.6 | 585 | 32.6 |
| | N | 3 | 3 | 3 | 3 | 3 | 3 | 3 |
| | Mean | 35.8 | 0.723 | 1570 | 1170 | 37.1 | 659 | 34.7 |
| | SD | 5.54 | 0.479 | 531 | 180 | 5.33 | 111 | 4.66 |
| | Median | 32.6 | 1.00 | 1460 | 1270 | 34.4 | 606 | 32.6 |
| HNSCC | ON1113 | 28.0 | 0.170 | 348 | 184 | 32.5 | 348 | 28.0 |
| | ON1114 | 34.1 | 0.170 | 1160 | 970 | 46.5 | 563 | 34.1 |
| | ON1118 | 29.9 | 1.00 | 1100 | 957 | 42.6 | 565 | 28.5 |
| | ON1119 | 29.2 | 0.500 | 747 | 520 | NR¹ | 520 | 27.5 |
| | ON1121 | 32.4 | 3.00 | 2690 | 1430 | NR¹ | 625 | 30.3 |
| | ON1125 | 47.6 | 0.170 | 2550 | 1480 | 38.4 | 730 | 47.6 |
| | ON1127 | 34.3 | 3.00 | 2740 | 1580 | 46.8 | 740 | 31.3 |
| | N | 7 | 7 | 7 | 7 | 5 | 7 | 7 |
| | Mean | 33.6 | 1.14 | 1620 | 1020 | 41.4 | 585 | 32.5 |
| | SD | 6.62 | 1.30 | 1010 | 524 | 6.01 | 134 | 7.05 |
| | Median | 32.4 | 0.500 | 1160 | 970 | 42.6 | 565 | 30.3 |
| ALL | N | 18 | 18 | 18 | 18 | 16 | 18 | 18 |
| | Mean | 34.3 | 0.834 | 1670 | 1100 | 44.5 | 615 | 33.0 |
| | SD | 4.97 | 0.862 | 904 | 413 | 16.3 | 104 | 5.34 |
| | Median | 33.6 | 0.500 | 1170 | 997 | 40.5 | 615 | 32.4 |

¹Not Reportable, R² < 0.8

The study was not powered to perform a statistical analysis for dose proportionality, but C10 appears to be dose proportional from 0.3 through 1.2 mg/kg (FIG. 6) and AUC₀₋₂₄ₕ appears to be dose proportional 1.2 mg/kg (FIG. 7).

### Example 5. Fluorescence Imaging Acquisition and Image Processing

Intraoperative images and videos of “open surgery” were obtained using either the NOVADAQ SPY Elite or the SurgVision Explorer Air. The distance of the camera to the tumor was approximately 20 cm for the Explorer Air and 30 cm for the NOVADAQ SPY, according to manual instructions. The NOVADAQ SPY camera was only able to make fluorescent videos, which could be converted to images during post-processing. The settings for raw data acquisition for this camera were fixed. For the Explorer Air, attempt was made to use the same settings (exposure time and gain) for each patient to allow direct comparison between the images obtained from both the systems, however, depending on the amount of fluorescence visible during surgery, adjustments were needed in some cases due to saturation of the camera system. In some patients, the Olympus NIR laparoscope and Da Vinci Firefly camera systems were used when no open surgery was performed. Systems were used according to the manufacturer's manual.

First, pre-excision fluorescence images and/or movies of the tumor and surrounding areas were made. After surgical excision, images of the wound bed were obtained. In cases where a fluorescence region was visible in the wound bed, a biopsy was taken when feasible, and the excised specimen imaged on all sides on the back table in the operating room. If applicable, lymph nodes were imaged when possible in situ and on the back table, after which the wound bed of the lymph node dissection was imaged again.

Designated imaging study staff performed fluorescence imaging. The surgeon was blinded to the pre-excision imaging to avoid any bias on standard surgery, but was able to look at a second monitor for white-light images while performing the surgical procedure. The surgeon assisted in the wound bed and back table imaging. During the imaging procedure, the ambient light in the surgical theatre was switched off to prevent possible interaction with the fluorescence imaging procedure itself.

Images were processed using Fiji (ImageJ, version 2.0.0). Images were scaled on a per-patient basis, based on the maximum and minimum fluorescent intensity per pixel.

### Intraoperative Back Table and Postoperative Imaging Acquisition

During all phases of tissue processing, the specimen was stored in the dark as much as possible to prevent possible photobleaching of the imaging agent.

Immediately after excision, the whole specimen was imaged on all 6 resection planes (e.g., frontal, dorsal, lateral, medial, caudal and cranial) using both the designated intraoperative camera system as well as the LI-COR PEARL® Trilogy system within a maximum duration of 60 minutes after surgical excision of the specimen (intraoperative back table imaging). Imaging time combined for both devices had a maximum of 5 minutes. Specimens were inked with blue and black ink to mark resection planes. The restriction in the use of 2 colors of ink did not affect the SOC for tissue processing by the pathologist, but if a third ink color was needed, green ink was used to define additional pathological resection margins of interest.

Timing of postoperative tissue slice imaging was adapted to accommodate the differences in the SOC for specimen processing of the different tumor types. Briefly, BC specimens were sliced fresh on the day of surgery and then formalin fixed, other tumor types were sliced after formalin fixation of the whole resection specimen 1 to 3 days after surgery. Generally, the surgical specimen was serially sliced into ±0.5 cm thick tissue slices. White light photographs were made during and directly after slicing for orientation purposes. After slicing, fluorescence imaging on both sides of each tissue slice was performed in a light-tight environment (LI-COR PEARL® Trilogy system). BC slices were therefore imaged approximately 120 min after excision, other tumor types were sliced and imaged the subsequent day(s) after excision and formalin fixation.

Each BLS underwent overnight formalin fixation in 4% paraformaldehyde/phosphate buffered saline. The pathologist then macroscopically sampled parts of BLS (FFPE embedding) for further analysis according to SOC and preparation of 4 μm slices for hematoxylin and eosin (H/E) staining to delineate tumor tissue for histopathological correlation. Additional FFPE blocks could be embedded based on fluorescence imaging of the BLS additional to the SOC examination by conventional macroscopic visual inspection of the pathologist. A standardized workflow was executed in order to cross-correlate final histopathology results with recorded fluorescence images of tissue slices of interest. FFPE blocks were scanned after 7 to 14 days using the Odyssey Flatbed Scanner (LI-COR Bioscience).

### Example 6. Histological Correlation

After the SOC pathological procedure was performed (approximately 7-10 days for Phase 1a and 7-14 days for Phase 1b), H/E slices were reviewed and discussed with the dedicated board-certified pathologist for the respective tumor type.

### Example 7. Postoperative Fluorescence Measurements

A correlation between HE slices and fluorescence images (i.e., bread loaf slice or BLS) were made using Adobe Illustrator and Fiji (ImageJ). After precisely and manually drawing the region of interest (ROI) containing tumor and background based on the histopathological outcome, a CNR was calculated for each LI-COR PEARL image of a separate BLS for each patient. The median CNR was calculated based on all available BLS containing tumor. The fluorescence measurements were performed using Fiji (ImageJ) for
Mean fluorescence intensity (MFI; fluorescent intensity per pixel)
Contrast (MFI of the tumor tissue)
Noise (MFI of tissue that does not contains tumor (e.g., healthy muscle, fibrosis, fat)
Standard deviation of the noise
CNR (contrast-to-noise ratio):

$$CNR = \frac{Fluorescence ( Tumor ) - Fluorescence ( Normal Tissue )}{Standard Deviation Fluorescence ( Normal Tissue )}$$

TBR (tumor-to-background fluorescence ratio):

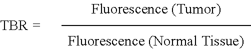

### Intraoperative Fluorescence Measurements

A macroscopic correlation between visible white light of the tumor areas and corresponding fluorescence images was made. After drawing ROIs containing macroscopic tumor and ROIs containing background, MFI of both tumor and background areas were calculated. Fluorescence ratios (CNR, TBR) were calculated on a per patient basis (3 measurements per patient) following the calculations described above.

### Example 8. Statistical Methods

Feasibility assessment of Compound 1 for intraoperative imaging of solid tumors and nodal metastasis included quantification of fluorescent signal CNR, sensitivity, and localization pattern of Compound 1 fluorescence. Furthermore, a range of safe doses corresponding to an adequate CNR was calculated by a combined assessment of intraoperative in vivo and ex vivo fluorescent signals (NOVADAQ imaging system) together with ex vivo examinations (e.g., histological examination, NIR flatbed scanning).

### Example 9. Patient Demographics and Specimen Characteristics

To evaluate the tumor agnostic imaging feasibility with Compound 1, 15 additional patients with 4 different tumor types (HNSCC, BC, EC, or CRC) were dosed in Phase 1b with an optimal dose of Compound 1 (1.2 mg/kg) chosen from Phase 1a. Patients in Phase 1b had HNSCC (n=5), BC (n=4), EC (n=3), and CRC (n=3). Specimen characteristics are presented in Table 11.

TABLE 11
Surgical/Pathology Specimen Characteristics

| | | | | | | | | | MFI | | |
|---|---|---|---|---|---|---|---|---|---|---|---|
| | | | | Max | LN | | FFPE | | Calculations | | |
| | Cohort | | | Tumor | Metastases | | Primary | FFPE | Based on | | Intraoperative |
| Patient | dose | | LN | Size | by | # of | Specimen | LN | # of | # of | Camera |
| ID | (mg/kg) | Stage | Disn | (cm) | Histology ^{a} | BLS | # | # | BLS | H/E | Used |

Phase 1a

| | | | | | | | | | | | |
|---|---|---|---|---|---|---|---|---|---|---|---|
| ON1101 | 0.3 | pT2N3aM1 | Yes | 3 | Yes (35/37) ^{a} | 12 | 25 | 44 | 3 | 14 | NOVADAQ |
| | mg/kg | | | | | | | | | | SPY |
| ON1102 | | pT1cN0 | No | 1.1 | No | 13 | 14 | 1 | 2 | 2 | NOVADAQ |
| | | | | | | | | | | | SPY |
| ON1103 | | pT2N0 | No | 2.5 | Yes (2/2), | 10 | 16 | 1 | 3 | 9 | NOVADAQ |
| | | | | | SNB | | | | | | SPY |
| ON1104 | 0.5 | pT4N1m0 | Yes | 3 | Yes (4/54) | 20 | 14 | 56 | 3 | 6 | NOVADAQ |
| | mg/kg | | Level 1-5 | | | | | | | | SPY |
| ON1105 | | pT2N0 | No | 2.8 | No (SNB) | 13 | 18 | 1 | 3 | 4 | NOVADAQ |
| | | | | | | | | | | | SPY |
| ON1106 | | pT3N2bM0 | Yes | 5 | Yes (2/16) | 13 | 13 | 21 | 4 | 6 | NOVADAQ |
| | | | Level 1-5 | | | | | | | | SPY |
| ON1107 | 0.8 | PT3N3a | Yes | 6.5 | Yes (16/26) | 14 | 24 | 33 | 3 | 18 | NOVADAQ |
| | mg/kg | | | | | | | | | | SPY |
| ON1108 | | pT3N0Mx | Yes | 3.2 | No (0/48) | 15 | 14 | 54 | 3 | 10 | NOVADAQ |
| | | | Level 1-3 | | | | | | | | SPY |
| ON1109 | | pT1cN0 | 1 extra | 1.1 | No (0/2), | 7 | 10 | 3 | 2 | 3 | SurgVision |
| | | | LN | | SNB | | | | | | Explorer Air |
| ON1110 | 0.1 | pT3N0mx | Yes | 2.3 | No (0/26) | 7 | 24 | 31 | 3 | 9 | SurgVision |
| | mg/kg | | | | | | | | | | Explorer Air |
| ON1111 | | pT1N0Mx | Level 2 | 2 | No (0/2) | 12 | 14 | 2 | 5 | 7 | SurgVision |
| | | | | | | | | | | | Explorer Air |
| ON1112 | | pT2N0 | No | 1.4 | No (0/1) | 20 | 11 | 2 | 4 | 4 | SurgVision |
| | | | | | SNB | | | | | | Explorer Air |
| ON1113 | 1.2 | pT3N2bMx | Yes | 4 | Yes (3/37) | 21 | 25 | 50 | 9 | 22 | SurgVision |
| | mg/kg | | | | | | | | | | Explorer Air |
| ON1114 | | PT1N1Mx | No | 2 | Yes (1/4) | 13 | 13 | 4 | 2 | 2 | SurgVision |
| | | | | | SNB | | | | | | Explorer Air |
| ON1115 | | pT1bN0Mx | No | 0.6 | No (0/1) | 11 | 28 | 2 | 4 | 7 | SurgVision |
| | | (2 primary | | | SNB | | | | | | Explorer Air |
| | | tumors) | | | | | | | | | |

Phase 1b

| | | | | | | | | | | | |
|---|---|---|---|---|---|---|---|---|---|---|---|
| ON1116 | 1.2 | pT1cN0 | No | 1.1 | No (0/2) | 11 | 47 | 3 | N/A | N/A | NOVADAQ |
| | mg/kg | | | | SNB | | | | | | SPY |
| ON1118 | | pT2N0 | Yes | 2.4 | No (0/15) | 25 | 21 | 31 | 6 | 12 | SurgVision |
| | | | | | | | | | | | Explorer Air |
| ON1119 | | pT4N0 | Yes | 3.8 | No (0/46) | 21 | 38 | 54 | 4 | 22 | SurgVision |
| | | | | | | | | | | | Explorer Air |
| ON1120 | | N/A | No | N/A | N/A | 2 | 2 | — | N/A | N/A | Olympus |
| | | | | | | | | | | | NIR |
| | | | | | | | | | | | Laparoscope |
| ON1121 | | pT1N0 | No | 0.5 | No (0/3) | 17 | 17 | 5 | 1 | 1 | SurgVision |
| | | | | | SNB | | | | | | Explorer Air |
| ON1122 | | ypT0N0 | Partial | N/A | No (0/16) | 45 | 71 | 15 | N/A | N/A | Da Vinci |
| | | | | | | | | | | | Robot Firefly |
| ON1123 | | pT1cN0 | No | 1.2 | No (0/1) | 11 | 16 | 2 | 2 | 8 | SurgVision |
| | | | | | SNB | | | | | | Explorer Air |
| ON1124 | | ypT3N1 | Partial | 5 | Yes (1/17) | 16 | 56 | 19 | 7 | 28 | Da Vinci |
| | | | | | | | | | | | Robot, |
| | | | | | | | | | | | Firefly |
| ON1125 | | pT1N0 | Partial | 1.7 | No (0/27) | 11 | 11 | 27 | 7 | 7 | SurgVision |
| | | | | | | | | | | | Explorer Air |
| ON1126 | | ypT2N2 | Partial | 5 | Yes (3/22) | 18 | 48 | 19 | 4 | 12 | Olympus |
| | | | | | | | | | | | NIR |
| | | | | | | | | | | | Laparoscope |
| ON1127 | | pT2N0 | No | 1.5 | No (0/5) | 12 | 6 | 5 | 4 | 4 | SurgVision |
| | | | | | | | | | | | Explorer Air |
| ON1128 | | pT3N0M0 + | No | 1.5 | No (0/1) | 9 | 21 | 1 | 2 | 2 | NOVADAQ |
| | | OC: no | | | SNB | | | | | | SPY |
| | | tumor^{b} | | | | | | | | | |
| ON1129 | 1.2 | ypT2N0 + | CRC: | CRC: | CRC: | CRC: | CRC: | CRC: | 3 | 19 | SurgVision |
| | mg/kg | pT3aN0 | partial | 1.5 | (0/20) | 46 | 47 | 2RC: | | | Explorer Air |
| | | | | RC^{b}: | RC: | RC: | RC: | 0 | | | |
| | | | | 12 | (0/2) | 60 | 29 | | | | |
| ON1130 | | pT4N1M1 | No | N/A | Yes | 2 | 2 | 0 | N/A | N/A | NOVADAQ |
| | | | | | | | | | | | SPY |
| ON1151 | | pT1b | No | 1 | No (0/1) | 12 | 13 | 1 | 5 | 8 | NOVADAQ |
| | | | | | SNB | | | | | | SPY |

^{a} Fraction indicates lymph nodes positive for tumor by pathology (e.g., 35/37 indicates that of the 37 lymph nodes removed, 35 were positive for tumor by pathology)
^{b}Patients ON1128 and ON1129 each had a second tumor type scheduled for surgery as indicated in addition to the tumor that was part of the study protocol. These tumor types were not used in imaging summaries or any quantitative analysis.

### Example 10. Fluorescence Imaging Results

### Phase 1a

Fluorescence imaging results for the completed Phase 1a dose-escalation portion of the Phase 1 study are available for all 15 patients; 3 patients each in Cohort 1 (0.3 mg/kg), Cohort 2 (0.5 mg/kg), Cohort 3 (0.8 mg/kg), Cohort 4 (0.1 mg/kg), and Cohort 5 (1.2 mg/kg) and for the 15 more patients in Phase 1b a 1.2 mg·kg.

### Fluorescence Images

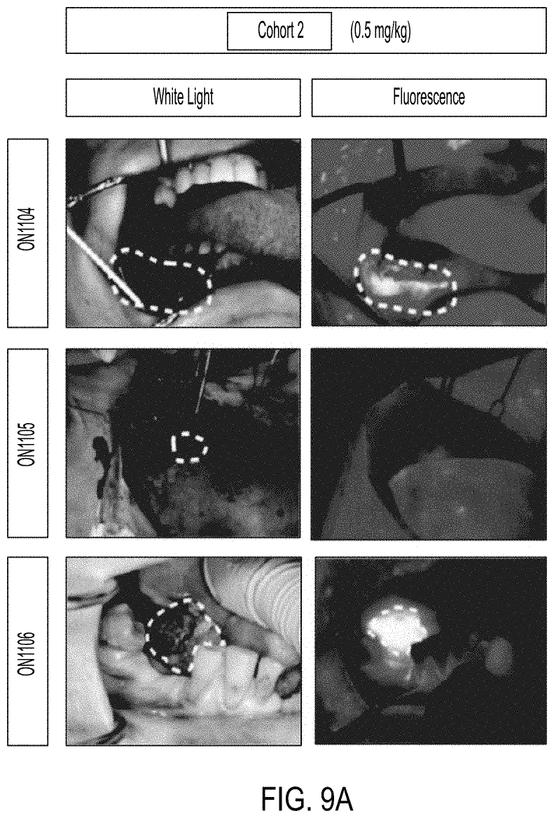

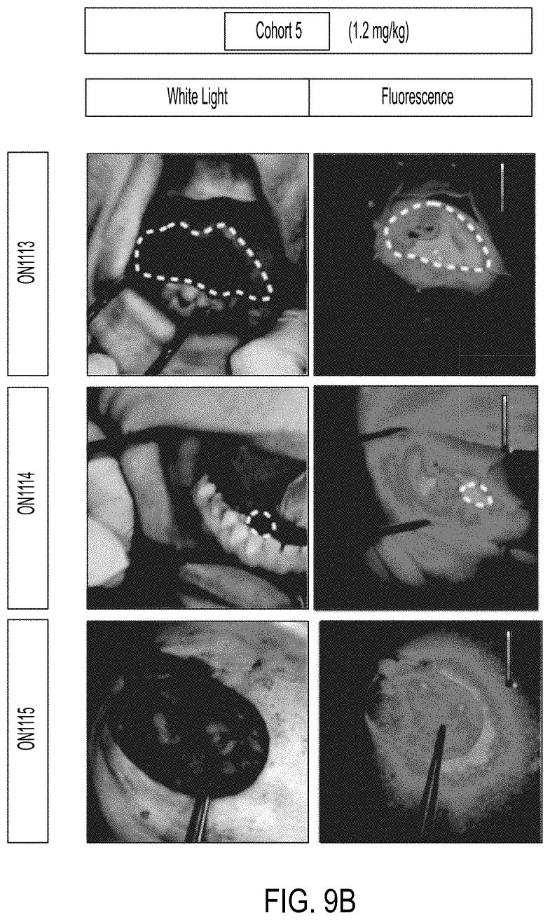

Intraoperative (FIG. 9A) and postoperative (FIG. 9B) images from 3 patients dosed in Cohort 2 (0.5 mg/kg) and Cohort 5 (1.2 mg/kg) are presented. In Cohort 2, Patients ON1104 and ON1106 had HNSCC and Patient ON1105 had breast cancer patient. In Cohort 5, Patients ON1113 and ON1114 had HNSCC and Patient ON1115 was a breast cancer patient.

Intraoperative imaging is defined as a combination of in vivo imaging and whole specimen back table imaging performed within an hour of surgery. Feasibility to image tumors with Compound 1 intraoperatively was clearly demonstrated in all 8 of the patients with HNSCC, who received Compound 1 between 0.1 and 1.2 mg/kg. Two of the 7 BC patient tumors were visualized with Compound 1. The remaining 5 BC tumors were deep seated and surrounded by normal tissue and were not visible intraoperatively by Compound 1 fluorescence imaging. This is not surprising due to limited tissue penetration with NIR imaging. Importantly, none of these 5 BC tumors had positive margins. These results clearly demonstrate feasibility for intraoperative imaging in HNSCC and BC with Compound 1.

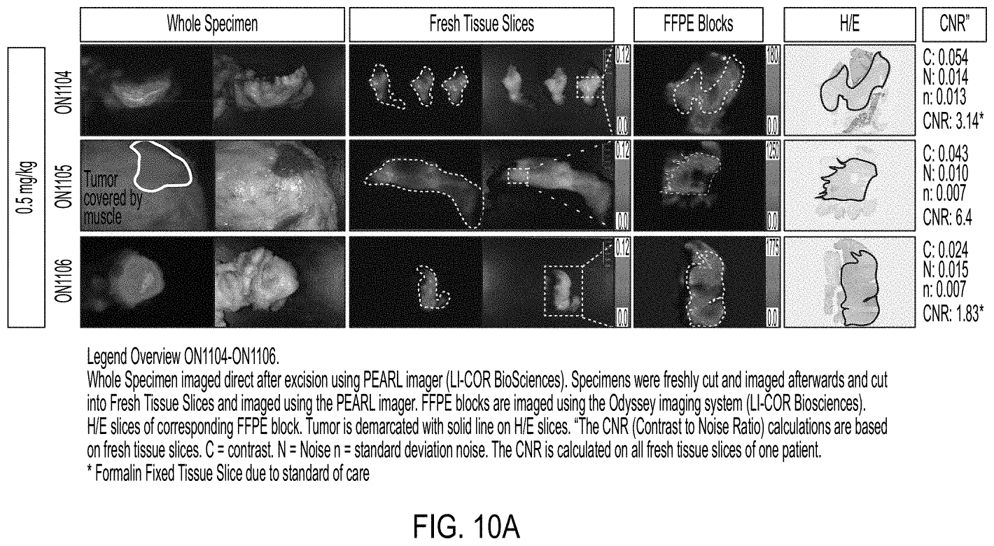

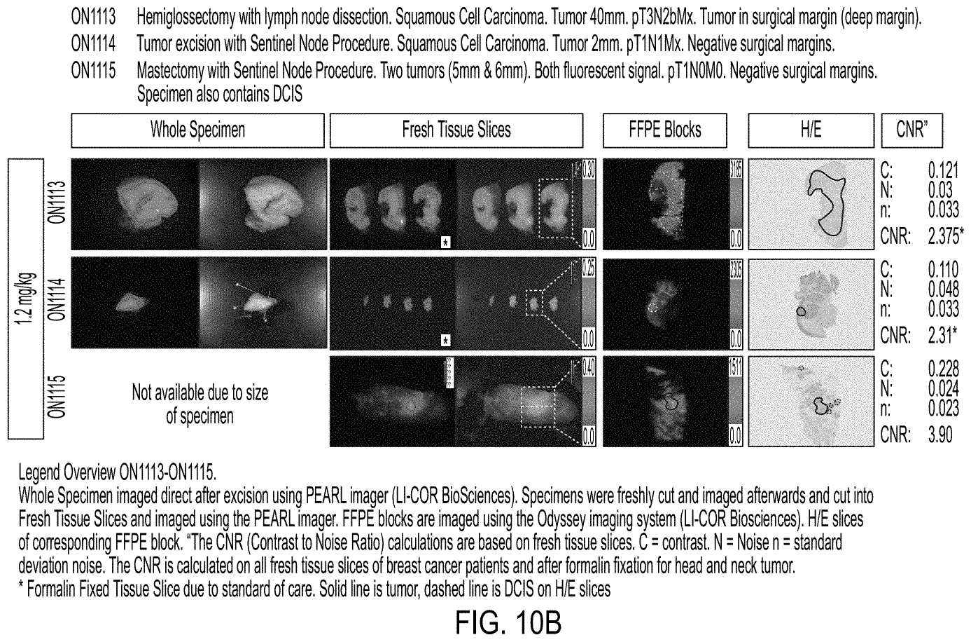

Postoperative tissue specimen imaging clearly shows Compound 1 imaging feasibility in all 15 patient tumor specimens. These images of Compound 1 show sharp boundaries between the bright fluorescent regions and the dark regions (FIGS. 10A, 15, and 16). The core of the tumors, which are necrotic, do not show fluorescence. For all patients, the fluorescent regions corresponded to the H/E images marked with the region of interest. High tumor to background fluorescence ratios (CNR, TBR) were seen from regions identified as tumor or normal based on histopathological correlation. Similar images were obtained for all 15 patients in Phase 1a at each dose level tested.

Mean Fluorescence Intensity Phase 1a—Primary Tumor

### Intraoperative Imaging

In Phase 1a, all (8 of 8) HNSCC patient tumors and 2 of 7 BC patient tumors (for whom macroscopic tumor was visible in vivo) showed Compound 1 fluorescence in vivo (dose range from 0.1 to 1.2 mg/kg). In all of these patients, MFI from the tumor tissue was above the MFI from the surrounding normal tissue.

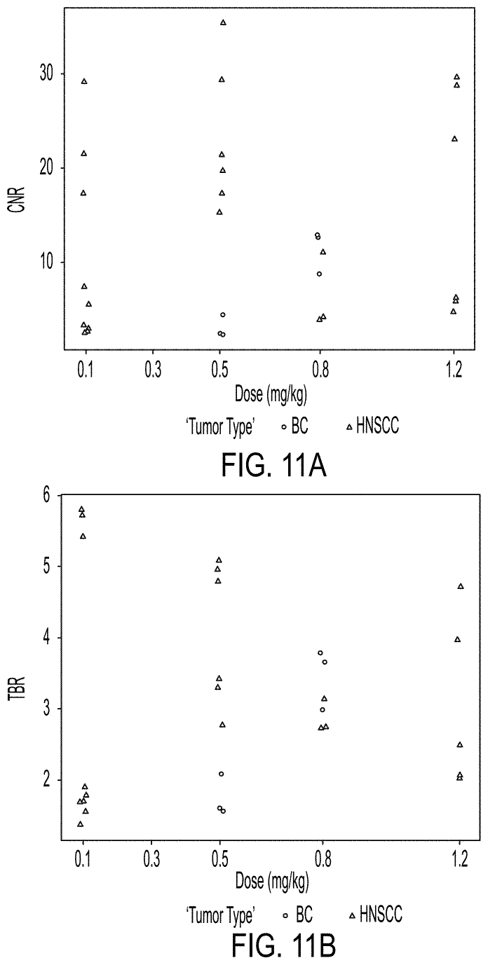

Intraoperative fluorescence intensity cannot be standardized and compared between patients or dose levels due to the unique presentation of each surgical environment. Multiple variables such as camera angle, camera-tissue distance, tumor location, and coverage by other tissues or fat affect the absolute value of the fluorescence signal. Hence, ratios of in vivo fluorescence from tumor and non-tumor tissues were calculated for each patient. As shown in FIG. 11A for CNR and FIG. 11B for TBR, the intraoperative TBR and CNR values were high for all the patients. This indicates the clear demarcation in fluorescence intensity between the tumor tissue and the normal tissue for individual surgeries, a key factor that could potentially aid the surgeon in real time visualization of tumors during surgical excision. These ratios were variable and did not show any systematic increase or decrease with dose.

### Postoperative Imaging

Compound 1 fluorescence images were captured from the postoperative specimens (BLS specimens) prepared at each step of the standard pathology for the purposes of correlating Compound 1 fluorescence with histopathological finding of the tumor and the normal tissue. LI COR PEARL, a laboratory camera with capabilities to standardize imaging and fluorescence quantification, was used to compare fluorescence intensity across multiple specimens.

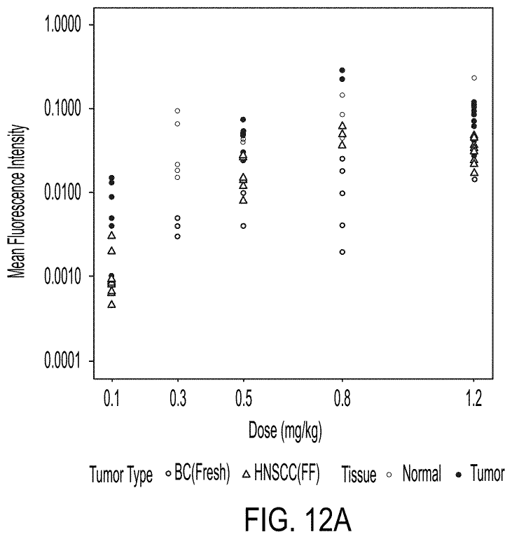

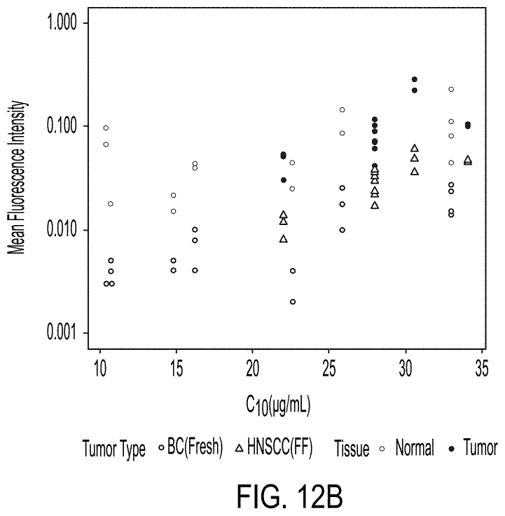

FIG. 12A shows the MFI from the histology confirmed tumor and normal tissue regions for multiple BLS selected by standard pathology for all 15 patients dosed at 5 dose levels (fresh samples from BC patients and formalin-fixed (FF) samples from NHSCC patients). Tumor MFI increased with dose. There was an increase in the normal tissue MFI with dose as well. When plotted against each patient's plasma concentration at 10 min. (FIG. 12B), MFI from histology confirmed tumor and normal tissue specimens showed clear demarcation (no overlap) for each patient (n=15). This is a key factor of importance for real time image guided surgery to aid surgeons to delineate tumor from background tissue. Similar to dose, MFI increased with increasing initial plasma concentration. These figures also show that there is no systematic trend in the fluorescence signals from formalin fixation-treated (FF) versus fresh tissues or between HNSCC versus BC tissues.

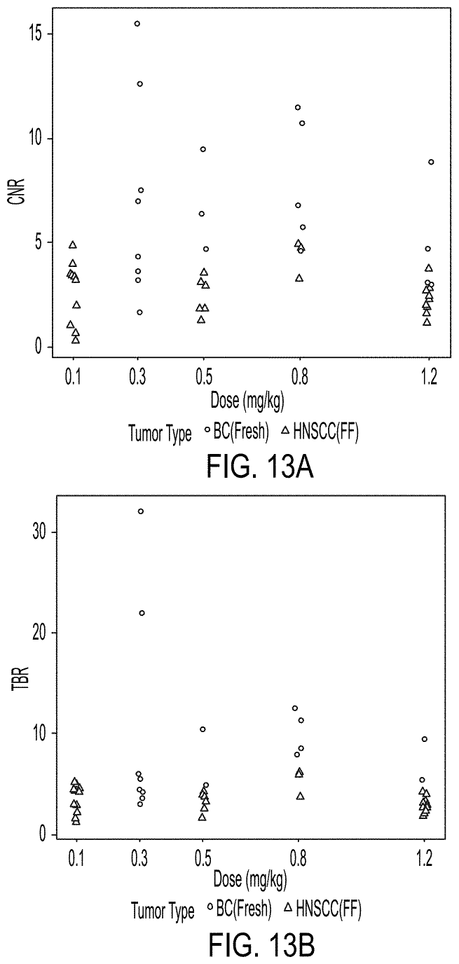

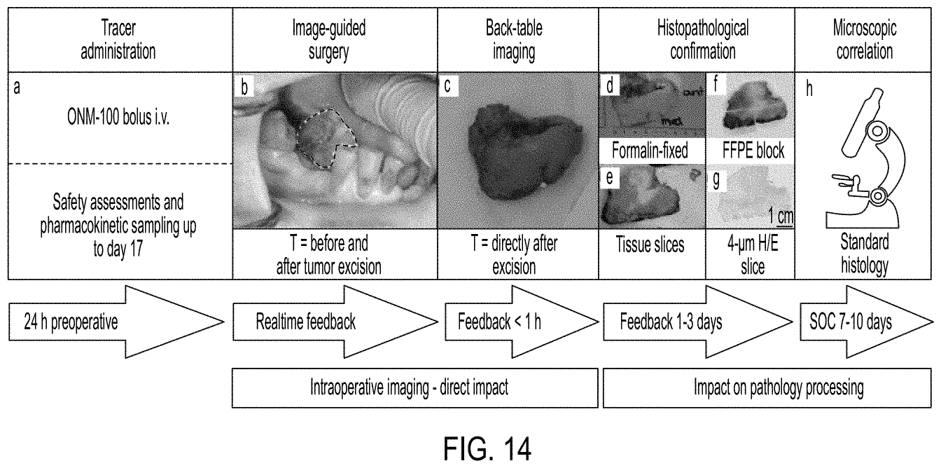

As with intraoperative imaging, TBR (FIG. 13A) and CNR (FIG. 13B) calculated using the postoperative fluorescence from the histology confirmed tumor and normal regions show high variability and remain relatively constant with dose.

### Phase 1A Summary

Intraoperative and postoperative fluorescence imaging was performed using open field and closed field NIR cameras after a single intravenous dose of Compound 1 administered 24±8 hours before surgery in 15 patients undergoing SOC BC or HNSCC cancer surgeries. Five (5) different dose levels were evaluated between the doses of 0.1 to 1.2 mg/kg. These data demonstrate feasibility to image tumors with Compound 1 in all HNSCC and BC patients. Compound 1 imaging was feasible with multiple NIR cameras that detect ICG. The MFI was well demarcated between tumor tissue and normal tissue for each patient. MFI for both tumor and normal tissue increased slightly in the dose range evaluated. The fluorescence ratios (CNR and TBR) were variable but high, further illustrating the sharp demarcation between the tumor and normal tissue fluorescence. CNR and TBR did not show any systematic increase or decrease with dose and was very similar for BC and HNSCC tumors.

The highest dose from the Phase 1a portion of the Phase 1 study (1.2 mg/kg) was selected for further evaluation of safety, PK, and imaging feasibility of Compound 1 in the Phase 1b portion of the study. In the Phase 1b portion of the study, 15 additional patients with 4 tumor types (BC, HNSCC, CRC, and EC) received Compound 1 (1.2 mg/kg) at a surgery/imaging time of 24±8 hours post dosing.

Selection of the 1.2 mg/kg dose level of Compound 1 for the Phase 1b portion of the study was based on the following results from the Phase 1a portion. In the Phase 1a study, the safety profile was comparable at all the dose levels studied and did not raise any specific safety concerns or trends at higher doses. Compound 1 plasma exposure increased proportionally with dose. Mean fluorescence intensity increased with Compound 1 plasma exposure; CNR and TBR values were variable, but remained high (in the range of 2-15) and did not decrease with dose for both in vivo tumor and postoperative specimen fluorescence. These data support using the highest feasible dose/exposure with higher fluorescence intensity for evaluating imaging feasibility with additional tumor types and endoscopic cameras and potentially other difficult scenarios such as tumors covered by normal tissues, tumor locations with anatomical challenges, ductal carcinoma in situ, multifocal tumors, and small lymph node metastases.

The highest dose from the Phase 1a portion of the Phase 1 study (1.2 mg/kg) was selected for further evaluation of safety, PK, and imaging feasibility of Compound 1 in the Phase 1b portion of the study. In the Phase 1b portion of the study, 15 additional patients with 4 tumor types (BC, HNSCC, CRC, and EC) received Compound 1 (1.2 mg/kg) at a surgery/imaging time of 24±8 hours post dosing.

### Fluorescence Images

Compound 1 fluoresced intraoperatively in 10/15 patient tumors that included 5 of 5 HNSCC, 3 of 4 BC, 2 of 3 CRC. One deeper seated rectal tumor and 1 deeper seated BC tumor could not be visualized intraoperatively, which is not surprising with NIR imaging due to limited penetration depth. Three (3) intraluminal EC tumors were not detected with extraluminal imaging (1 EC patient had pathological complete response). As in Phase 1a, Compound 1 fluorescence detected all positive margin BC and HNSCC patients in Phase 1b. None of the intraluminal or deep-seated tumors had a positive margin on final pathology. As in Phase 1a, postoperatively Compound 1 fluoresced in tissue slices from all the patients and tumor types (including 2 of the 3 EC patients with viable tumor). Postoperative images clearly show the sharp boundaries between the bright fluorescent regions and the blue/dark region.

Phase 1b confirms imaging feasibility in BC and HNSCC (as in Phase 1a) and demonstrates imaging feasibility in other solid tumors with the similar sharp boundaries between tumor and normal tissue.

Mean Fluorescence Intensity Phase 1a and Phase 1b—Primary Tumor

### Intraoperative Imaging

In the following analysis, data from all of the patients dosed at 1.2 mg/kg in Phase 1a and Phase 1b are combined. A total of 18 patients with HNSCC (n=7), BC (n=5), EC (n=3), and CRC (n=3) received Compound 1 at 1.2 mg/kg in Phase 1a and Phase 1b.

Intraoperative MFI, CNR and TBR were calculated for those patient tumors for whom intraoperative imaging was feasible (11 of 18 patient tumors, see Table 15). Intraoperative CNR and TBR values were high for all the patients, indicating the clear demarcation in fluorescence intensity between the tumor tissue and the normal tissue for individual surgeries. This is a key factor of importance that could potentially aid the surgeon in real time delineation of tumors from background during surgical excision. These CNR and TBR results also indicate that Phase 1b results are confirmatory of Phase 1a results.

### Summary of Phase 1b Results

The data from Phase 1b clearly shows that Compound 1 was well tolerated at a dosage of 1.2 mg/kg and allowed fluorescent tumor visualization both intraoperatively and postoperatively in BC, HNSCC, CRC, peritoneal metastasis, and possibly EC (as shown by postoperative imaging), supporting the tumor agnostic mechanism of action of Compound 1 for solid pan-tumor imaging.
Peritoneal metastasis was visualized in 2 patients using Compound 1 (NOVADAQ and Olympus and PEARL) as well as extraluminal CRC (NOVADAQ).
Ductal carcinoma in situ (DCIS) in patients with BC could be detected with Compound 1, both in vivo and back table, indicating towards intraoperative guidance and decision making.
Lobular carcinoma (and lobular carcinoma in situ) in patients with BC was detected by Compound 1.
Margin assessment on specimens directly after excision seems feasible using Compound 1 (both on whole specimen and BLS).

The value of intraoperative imaging EC using Compound 1 could not be evaluated due to the fact that no images could be collected intraoperatively due to the lack of sensitivity of the minimal invasive camera systems. However, EC tissue slices were visualized with Compound 1 imaging with LI-COR Pearl camera. Optimizing Compound 1 dose/schedule for imaging as well improvements in camera technologies may overcome this limitation.

Phase 1b portion of the Phase 1 study further confirmed Compound 1 imaging feasibility with multiple NIR cameras designed to detect ICG.

Intraoperative fluorescent imaging with Compound 1 is clinically feasible at a dosage of 1.2 mg/kg for both SurgVision Open Air and NOVADAQ SPY Elite fluorescence cameras.

Intraoperative visualization of tumors with Compound 1 using the Olympus Fluorescent Laparoscope and the DaVinci Robot with firefly camera was challenging, as the sensitivity of both cameras is lower compared to the SurgVision and the NOVADAQ SPY. A higher dose may be needed for optimal imaging performance.

### Example 11. Lymph Node Imaging

After lymph node dissection, lymph nodes were identified by the attending pathologist and harvested if present. After harvesting, the single lymph nodes were imaged before further processing using PEARL imaging. The images were processed using ImageJ (FiJi). The fluorescent images were reviewed by 2 separate researchers blinded for histology, whether fluorescence was present. The pathologist, blinded for fluorescence images, evaluated whether the lymph node was positive for tumor invasion or isolated tumor cells based on H/E staining.

By-patient results are presented in Table 12. Of the 403 available lymph nodes from patients undergoing lymphadenectomy across the 4 tumor types, 64 contained pathology confirmed tumors (35 from a single patient) of which Compound 1 fluoresced in 30 lymph nodes. Compound 1 accurately did not fluoresce in 293 of 339 pathology negative lymph nodes.

TABLE 12
Performance characteristics of Compound
1 in detecting lymph node metastases

| | True | True | False | False | Total |
|---|---|---|---|---|---|
| Patient | Positives | Negatives | Positives | Negatives | Samples |
| ID | n | n | n | n | n |
| ON1101 | 10 | 2 | 0 | 25 | 37 |
| ON1102 | 0 | 0 | 1 | 0 | 1 |
| ON1103 | 2 | 0 | 0 | 0 | 2 |
| ON1104 | 0 | 50 | 3 | 1 | 54 |
| ON1105 | 0 | 0 | 1 | 0 | 1 |
| ON1106 | 1 | 14 | 0 | 1 | 16 |
| ON1107 | 12 | 7 | 3 | 3 | 25 |
| ON1108 | 0 | 44 | 4 | 0 | 48 |
| ON1109 | 0 | 0 | 2 | 0 | 2 |
| ON1110 | 0 | 20 | 6 | 0 | 26 |
| ON1111 | 0 | 2 | 0 | 0 | 2 |
| ON1112 | 0 | 0 | 1 | 0 | 1 |
| ON1113 | 2 | 34 | 0 | 1 | 37 |
| ON1114 | 1 | 0 | 3 | 0 | 4 |
| ON1115 | 0 | 1 | 0 | 0 | 1 |
| ON1116 | 0 | 2 | 0 | 0 | 2 |
| ON1118 | 0 | 15 | 0 | 0 | 15 |
| ON1119 | 0 | 41 | 1 | 0 | 42 |
| ON1120 | 0 | 0 | 0 | 0 | 0 |
| ON1121 | 0 | 1 | 2 | 0 | 3 |
| ON1122 | 0 | 8 | 3 | 0 | 11 |
| ON1123 | 0 | 0 | 1 | 0 | 1 |
| ON1124 | 1 | 14 | 1 | 0 | 16 |
| ON1125 | 0 | 16 | 11 | 0 | 27 |
| ON1126 | 1 | 18 | 1 | 2 | 22 |
| ON1127 | 0 | 4 | 1 | 0 | 5 |
| ON1128 | 0 | 0 | 0 | 1 | 1 |
| ON1129 | 0 | 0 | 0 | 0 | 0 |
| ON1130 | 0 | 0 | 0 | 0 | 0 |
| ON1151 | 0 | 0 | 1 | 0 | 1 |
| TOTAL | 30 | 293 | 46 | 34 | 403 |

ID = identification

Overall performance characteristics are presented in Table 13.

Overall sensitivity of Compound 1=(true positive)/(true positive+false negative) =30/(30+34)=0.47.

Overall specificity of Compound 1=(true negative)/(false positive+true negative)=293/(46+293)=0.86.

TABLE 13
Overall sensitivity and specificity of Compound 1 in lymph nodes

| Result by Compound 1 | Result by H/E Staining | | |
|---|---|---|---|
| Fluorescence Imaging | Positive | Negative | TOTAL |
| Positive | 30 | 46 | 76 |
| Negative | 34 | 293 | 327 |
| TOTAL | 64 | 339 | 403 |

H/E = haemotoxylin and eosin

Accurate intraoperative detection of metastatic lymph nodes is a high unmet need and technologically challenging. It is hypothesized that at imaging times >24 hours, there is likely nonspecific fluorescence in the lymph nodes due to the primary tumor fluorescence draining into the lymph nodes. Low sensitivity may be due to the relatively small amount of Compound 1 in the metastatic lymph nodes due to the small size of the lymph nodes (i.e., less absolute fluorescence) compared to primary tumors. Thus, higher doses at earlier imaging times may provide improved diagnostic performance for Compound 1 fluorescence imaging of primary tumors and metastatic lymph nodes.

### Example 12. Compound 1 Fluorescence Imaging—Clinical Utility

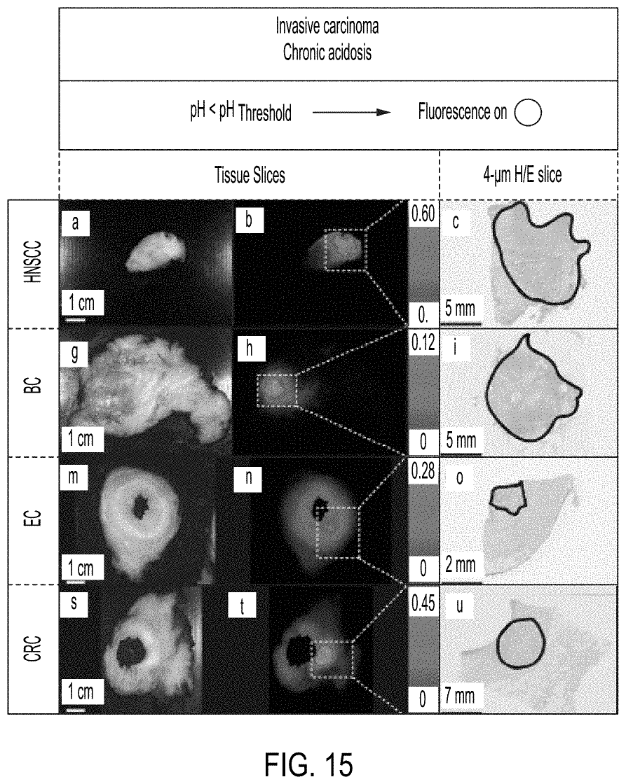

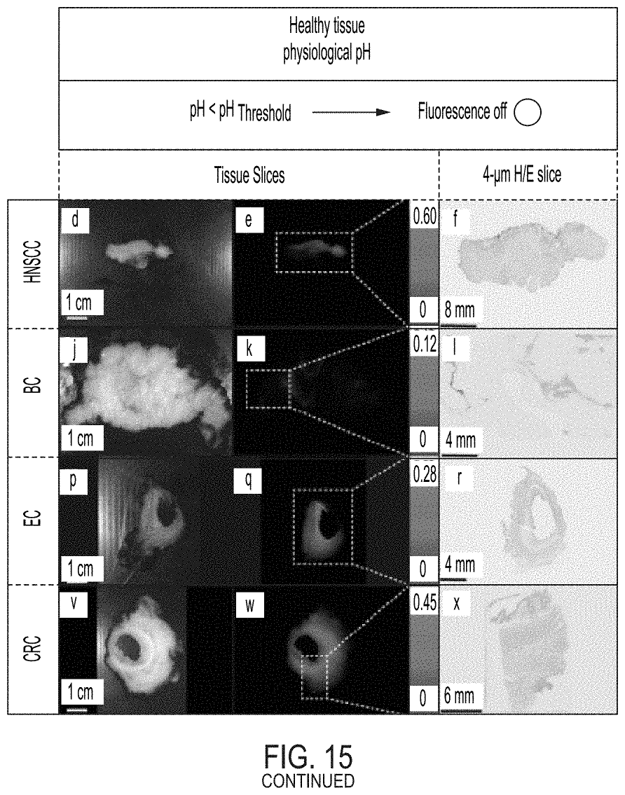

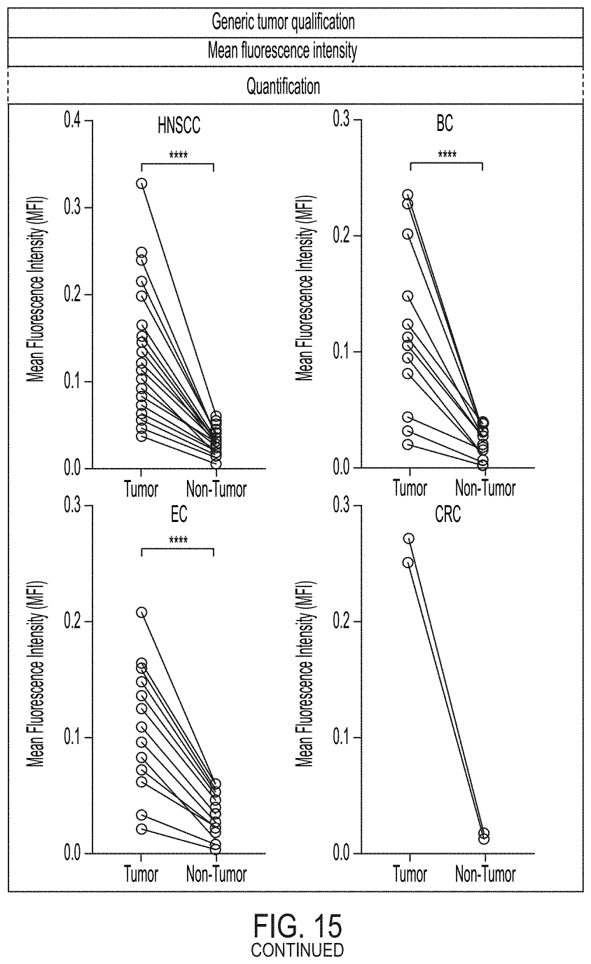

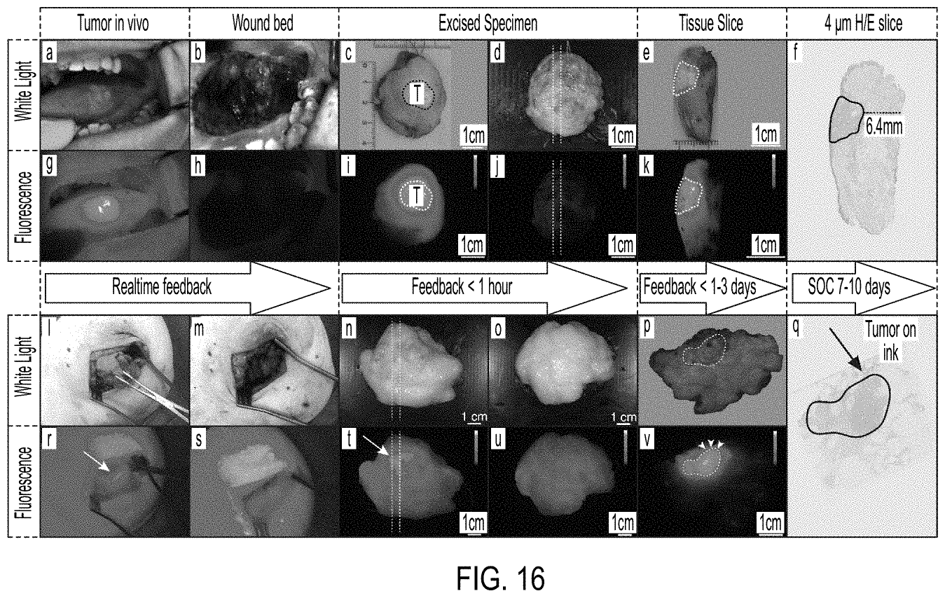

Fluorescence imaging with Compound 1 was feasible in all patients with viable tumors (29 out of 30 patients) and for all 4 tumor types evaluated (HNSCC, BC, CRC, or EC), FIGS. 15 and 16. Intraoperatively (combination of in vivo and back table imaging within 1 hour of surgery), all 13 HNSCC tumors, 5 of 11 superficially seated BC tumors, and 2 of 3 CRC tumors could be visualized by Compound 1 fluorescence. 6 of 11 deeper seated BC tumors, 2 of 3 intraluminal EC (1 of 3 EC was confirmed to have pathological complete response) and 1 of 3 CRC (distant rectal tumor) tumor could not be visualized. Absence of intraoperative fluorescence in some of these settings is likely due to the limits of NIR penetration depth when tumor is covered by normal tissue, lower sensitivity of current robotic and endoscopic cameras, optimal dose/schedule and physical challenges to access certain anatomical locations. Notably, none of these intraluminal or deep-seated tumors had a positive margin on final histopathology.

Postoperatively, all tumors irrespective of tumor type or dose were fluorescent upon the standard, fluorescent, postoperative workflow analysis, while none of the healthy tissue specimens were fluorescent.

FIG. 11A-11B, and quantitative fluorescence data, clearly show that the tumor fluorescence is well demarcated from the background fluorescence. This ability of Compound 1 to help visualize tumors with a sharp delineation from the normal tissue for the 4 tumor types evaluated across multiple patients establishes the tumor agnostic imaging feasibility for Compound 1 image-guided surgery in solid cancers.

### Fluorescence Detection of Tumor Positive Margins

In a total of 24 patients (HNSCC:13; BC:11), 9 patients (HNSCC:6; BC:3) had histologically confirmed tumor positive surgical margins that were undetected during SOC surgery. Fluorescence guided margin assessment was performed on a per-patient basis. Compound 1 imaging visualized all of these surgical margin patients yielding 100% sensitivity. All fluorescence-negative surgical margins correlated with final histopathological assessment (no false negatives). There were 5 of 15 false positives (specificity of 67%), in which fluorescence detected tissue was not confirmed to be tumor by histopathological assessment. 5 of 14 (36%) patients had fluorescent tissues that were negative for tumor (PPV: 64%).

By tumor type, sensitivity and specificity of Compound 1 for detecting positive margin patients were respectively 100% and 75% for BC and 100% and 57% for HNSCC. In 2 of 3 EC and 1 of 3 CRC patients for whom histological margin status was available and was negative, Compound 1 fluorescence was negative. These preliminary data suggest tumor agnostic diagnostic performance and demonstrate feasibility for accurate detection of tumor positive margins during surgery using Compound 1 imaging.

Table 14 summarizes the pathology versus fluorescence correlation for the margin status for individual patients for all 4 tumor types.

TABLE 14
Compound 1 fluorescence correlation with surgical margin status

| | | | Closest | | | |
|---|---|---|---|---|---|---|
| | | | Tumor | Surgical Margin | Surgical | Histology vs. |
| | | Patient | Positive | by Histology for | Margin by | Fluorescence |
| Patient | Dose | Group | Margin | Primary Tumor^{b} | Fluorescence^{c} | Correlation |
| ON1101 | 0.3 | BC | >5 mm | Free | Negative | TN |
| ON1102 | mg/kg | BC | >5 mm | Free | Negative | TN |
| ON1103 | | BC | cut through^{a.} | More than focal | Positive | TP |
| | | | | not free (Positive) | | |
| ON1104 | 0.5 | HNSCC | 3.5 mm | Close margin | Negative | TN |
| ON1105 | mg/kg | BC | <1 mm | free | Negative | TN |
| ON1106 | | HNSCC | cut through | Positive in medial | Positive | TP |
| ON1107 | 0.8 | BC | >5 mm | Free | Negative | TN |
| ON1108 | mg/kg | HNSCC | 3 mm | Close margin in | Positive | FP |
| | | | | basal and caudal | | |
| ON1109 | | BC | cut through | Focal not free (Positive) | Positive | TP |
| ON1110 | 0.1 | HNSCC | <1 mm | Positive in dorsal | Positive | TP |
| ON1111 | mg/kg | HNSCC | cut through | Positive in | Positive | TP |
| | | | | medial and basal | | |
| ON1112 | | HNSCC | 3 mm | Close margin | Negative | TN |
| | | | | in anterior | | |
| ON1113 | 1.2 | HNSCC | <1 mm | Positive in | Positive | TP |
| | mg/kg | | with | deep margin | | |
| | | | extra | | | |
| | | | satellite | | | |
| | | | lesions | | | |
| ON1114 | | HNSCC | >5 mm | Free | Positive | FP |
| ON1115 | | BC 2x | >5 mm | Free | Negative | TN |
| ON1116 | | BC | >5 mm | Free | Negative | TN |
| ON1118 | | HNSCC | <1 mm | Positive in medial | Positive | TP |
| ON1119 | | HNSCC | cut through | Positive in | Positive | TP |
| | | | | posterior, medial | | |
| ON1120 | | CRC | N/A | N/A | N/A | N/A |
| | | (PM) | | | | |
| ON1121 | | HNSCC | 3 mm | Close in basal | Positive | FP |
| ON1122 | | EC | N/A | N/A | N/A | N/A |
| ON1123 | | BC | >5 mm | Free | Positive | FP |
| ON1124 | | EC | >5 mm | Free | Negative | TN |
| ON1125 | | HNSCC | 2 mm | Close margin | Negative | TN |
| | | | | in cranial, | | |
| | | | | caudal, anterior | | |
| ON1126 | | EC | >5 mm | Free | Negative | TN |
| ON1127 | | HNSCC | >5 mm | Free | Negative | TN |
| ON1128 | | BC | cut through | Positive in ventral | Positive | TP |
| ON1129 | | CRC | >5 mm | Free | Negative | TN |
| ON1130 | | CRC | N/A | N/A | N/A | N/A |
| ON1151 | | BC | 3 mm | Close in cranial | Positive | FP |
| | | (DCIS) | | | | |

BC = breast cancer;
CRC = colorectal cancer;
DOS = ductal carcinoma in situ;
EC = esophageal cancer;
HNSCC = head and neck squamous cell carcinoma;
N/A = not applicable;
PM = peritoneal metastasis;
TP = true positive;
TN = true negative, FP = false positive
^{a.}“Cut through’’ refers to when closest tumor positive margin is 0 mm.
^{b}Surgical margin status is based on Dutch Guidelines
^{c}Postoperative whole specimen imaging was performed using intraoperative cameras at the back table and with LI-COR PEARL Trilogy within 1 hour of surgical excision. Margin assessment was done by combining fluorescence data from cavity fluorescence and specimen margin fluorescence

### False Positive Fluorescence Margins

In 3 HNSCC patients (ON1108, ON1114, ON1121) and 2 BC patients (ON1123, and ON1151), false positive fluorescence margins were detected that did not contain tumor by final histopathological examination. In HNSCC patients, false positive fluorescence corresponded to a nerve tissue in 1 patient, salivary gland in another and a fluorescent spot on the specimen margin of the third patient. In the 2 BC patients, the false positive fluorescence margins corresponded to major fascia of pectoralis muscle, and DCIS tissue that was histologically classified as negative margin.

Compound 1 fluorescence was clearly detected in skin intraoperatively, as well as in ex-vivo specimens, of mastectomy patients. In mastectomy patients, Compound 1 fluorescence was observed in the nipple.

### Example 13. Compound 1 Detection of Occult Disease

Compound 1 fluorescence detected 5 additional occult lesions (1 patient with HNSCC and 4 patients with BC) otherwise missed by SOC preoperative surgery or during surgery or postoperative pathology. In 1 patient with HNSCC (ON1113) who had both a fluorescent and histopathological positive surgical margin, a satellite metastasis that was otherwise undetected by standard-of-care surgery was detected in the wound bed by Compound 1 fluorescence image-guided surgery.

One BC patient (ON1151) with both wound bed and back table specimen margin fluorescence categorized as a false positive result (i.e., histopathological negative margin as defined by the Society of Surgical Oncology and the American Society for Radiation Oncology guidelines), fluorescence corresponded to DCIS, an entity with cancer cells within the wall of the ductuli, and by international guidelines might require additional surgery, underscoring the clinical utility of detecting this lesion.

In 3 other BC patients, fluorescence imaging during histopathological processing detected additional otherwise missed cancers. Of these, patients ON1101 and ON1128 had an additional satellite metastasis of BC in the tissue slices detected by Compound 1. In patient ON1115, Compound 1 detected a second primary tumor lesion (triple-negative BC), missed during the preoperative work-up and surgery.

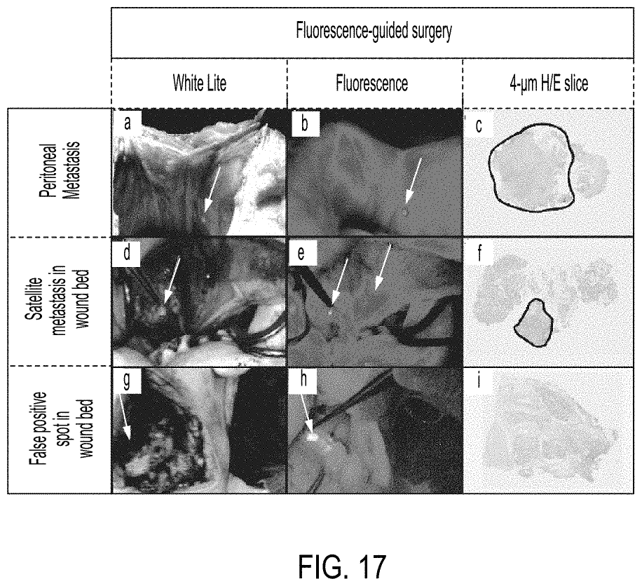

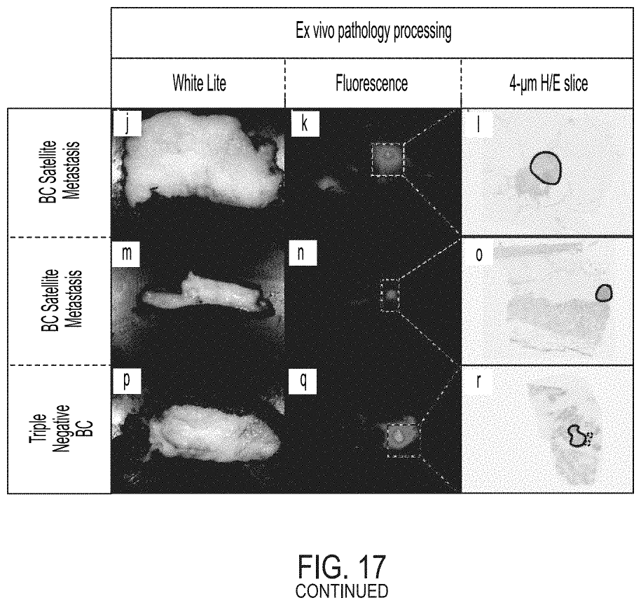

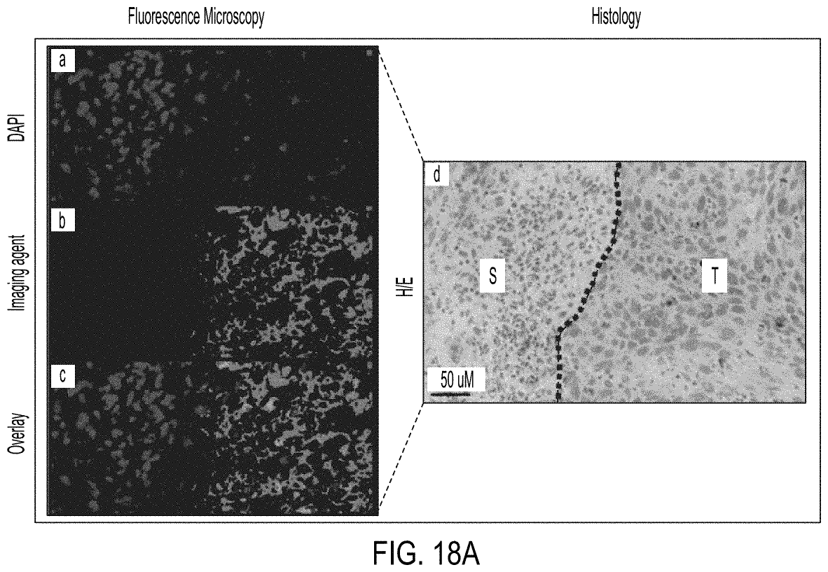

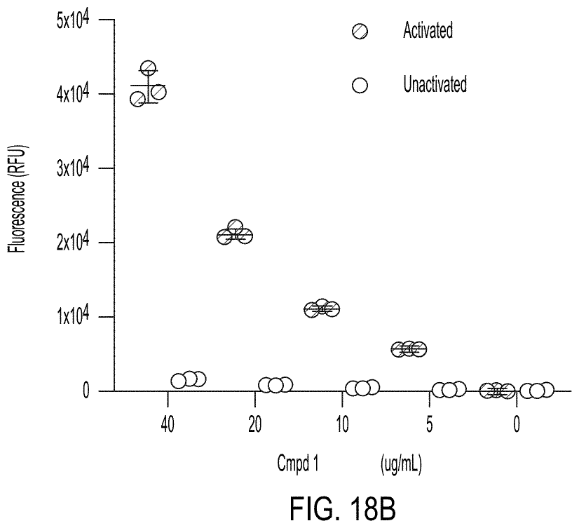

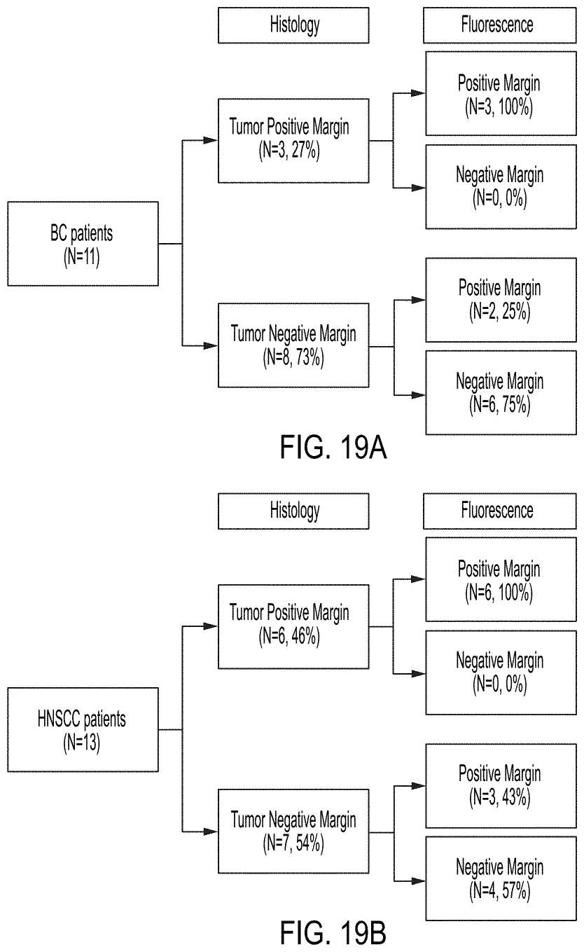

Of the 3 patients with CRC, the surgeon detected unexpected peritoneal metastases during surgery and per SOC procedures for 1 patient (ON1130). A second CRC patient presented with an already preoperative clinical suspicion of peritoneal metastases (ON1120). In both patients, the peritoneal metastases were fluorescent tumor-positive lesions (FIG. 17) and confirmed to be malignant by final histopathology.

The ability to detect tumor positive margins and occult disease across tumor types with similar high sensitivity and specificity underscores the significant potential for Compound 1 image-guided surgery to aid in clinical decision making for operative and postoperative patient management.

### Compound 1 Diagnostic Performance

In this Phase 1 study, the intraoperative and postoperative imaging data was used for a preliminary analysis of the diagnostic performance of Compound 1. Performance parameters such as MFI, CNR, and TBR were calculated in vivo and in tissue slices to characterize the ability of Compound 1 to delineate tumor tissue from background. Sensitivity and specificity for detecting tumor tissue from the adjacent normal tissue was evaluated using tissue specimen fluorescence and presented as a ROC curve. The sensitivity, specificity, and PPV of Compound 1 fluorescence in detecting pathology confirmed tumor positive margins were obtained at the patient level.

Table 15 summarizes in vivo and ex vivo CNR and TBR values for all tumor types and patients for whom in vivo imaging was feasible or for whom tissue slices were available to allow fluorescence quantification. These ratios were variable, but high, indicating that MFI of tumor tissue was always higher than that of background tissue, an important factor for fluorescence-guided surgery. The CNR and TBR values did not show any systematic variation with dose or tumor type.

### Intraoperative In Vivo CNR and TBR

In vivo CNR and TBR values at 1.2 mg/kg were high across all tumor types for all patients for whom in vivo imaging was feasible (11 of the 18 patients: HNSCC, 7 of 7; BC, 3 of 5; EC, 0 of 3; and CRC, 1 of 3). Using only the mucosal tumors (HNSCC) that were directly exposed to the surface where reliable evaluation was feasible, median CNR at 1.2 mg/kg was 5.6 with an interquartile range of 17.6 and median TBR of 2.6, with an interquartile range of 1.4. These high CNR and TBR ratios signify the sharp delineation of the tumor tissue from the background tissue for each patient's surgery, a key requirement for accurate image-guided surgery.

### Intraoperative Diagnostic Performance in Detecting Surgical Margin

Compound 1 showed 100% sensitivity with no false negatives in detecting tumor positive surgical margin patients. Specificity and PPV of Compound 1 for detecting surgical margin patients were 67% and 64%, respectively. By tumor type, sensitivity and specificity of Compound 1 for detecting surgical margin patients were 100% and 75% for BC and 100% and 57% for HNSCC, respectively. In 2 of 3 EC and 1 of 3 CRC patients for whom histological margin status was available and was negative, Compound 1 fluorescence was negative. These preliminary data suggest tumor agnostic diagnostic performance and demonstrate feasibility for accurate detection of tumor positive margins during surgery using Compound 1 imaging.

TABLE 15
CNR and TBR fluorescence ratio values for intraoperative in vivo
imaging with open field cameras and postoperative tissue slice
imaging with LI-COR Pearl closed-field camera by tumor type

| | | | | | | Ex Vivo | Ex Vivo | |
|---|---|---|---|---|---|---|---|---|
| | | | | | Number | Tissue | Tissue | Number |
| | Dose | Tumor | In Vivo | In Vivo | of in vivo | Slice | Slice | of tissue |
| Patient # | mg/kg | Type | CNR | TBR | images | CNR | TBR | slices |
| ON1104 | 0.5 | HNSCC | 17.3 | 3.3 | 3 | 3.1 | 3.9 | 3 |
| ON1106 | | | 29.3 | 5.0 | 3 | 1.8 | 2.2 | 4 |
| ON1108 | 0.8 | | 4.1 | 2.7 | 3 | 4.8 | 5.9 | 3 |
| ON1110 | 0.1 | | 2.9 | 1.7 | 3 | 3.5 | 4.5 | 3 |
| ON1111 | | | 21.5 | 5.7 | 3 | 3.2 | 4.5 | 5 |
| ON1112 | | | 3.3 | 1.7 | 3 | 0.9 | 1.9 | 4 |
| ON1113 | 1.2 | | 28.7 | 4.7 | 3 | 2.4 | 2.9 | 9 |
| ON1114 | | | 5.8 | 2.1 | 3 | 1.9 | 2.2 | 2 |
| ON1118 | | | 20.8 | 2.9 | 3 | 3.5 | 5.4 | 6 |
| ON1119 | | | 29.9 | 5.4 | 3 | 2.8 | 3.7 | 4 |
| ON1121 | | | 3.4 | 2.3 | 3 | 1.9 | 2.5 | 1 |
| ON1125 | | | 2.8 | 1.8 | 3 | 3.5 | 6.5 | 7 |
| ON1127 | | | 3.5 | 2.7 | 3 | 4.4 | 5.2 | 4 |
| ON1101 | 0.3 | BC | N/A | N/A | — | 7.0 | 4.5 | 3 |
| ON1102 | | | N/A | N/A | — | 14.1 | 27.0 | 2 |
| ON1103 | | | N/A | N/A | — | 3.2 | 4.2 | 3 |
| ON1105 | 0.5 | | 2.5 | 1.6 | 3 | 6.4 | 5.0 | 3 |
| ON1107 | 0.8 | | N/A | N/A | — | 5.7 | 8.0 | 3 |
| ON1109 | | | 12.7 | 3.7 | 3 | 9.2 | 11.9 | 2 |
| ON1115 | 1.2 | | N/A | N/A | — | 3.9 | 4.8 | 4 |
| ON1116 | | | N/A | N/A | — | N/A | N/A | — |
| ON1123 | | | 7.8 | 3.9 | 3 | 10.5 | 14.2 | 2 |
| ON1128 | | | 9.2 | 3.4 | 3 | 7.6 | 12.0 | 2 |
| ON1151 | | | 1.3 | 1.2 | 3 | 3.4 | 4.2 | 4 |
| ON1126 | 1.2 | EC | N/A | N/A | — | 2.9 | 3.4 | 4 |
| ON1122 | | | N/A | N/A | — | N/A | N/A | — |
| ON1124 | | | N/A | N/A | — | 2.1 | 3.2 | 7 |
| ON1129 | | CRC | N/A | N/A | — | 12.8 | 16.9 | 3 |
| ON1130 | | | 7.4 | 2.4 | 3 | N/A | N/A | — |
| ON1120 | | CRC-PM | N/A | N/A | — | N/A | N/A | — |

Note:
N/A: In vivo imaging was not feasible in 5 of 11 BC patients, 3 of 3 EC patients, and 2 of 3 CRC patients. Postoperative CNR/TBR calculation were not feasible in 4 of 30 patients, “n” refers to number of in vivo images used per patient and number of tissue slices used per patient for CNR and TBR calculations respectively for in vivo imaging and tissue slice imaging. Patients ON1116, ON1120, ON1122, ON1130 had no tissue slices: ON1116 (BC): not enough specimen due to small tumor surrounded by a lot of DCIS , ON1120 (CRC-PM): not enough specimen due to PM biopsy, ON1122 (EC): no tumor due to complete response, ON1130 (CRC): not enough specimen, no negative control.

### Postoperative MFI, CNR, TBR, and ROC Curve

Intraoperative fluorescence intensity cannot be standardized and compared between patients or dose levels due to the unique presentation of each surgical environment. Multiple variables such as camera angle, camera-tissue distance, tumor location, and coverage by other tissues or fat affect the absolute value of the fluorescence signal. To enable direct comparison of MFI across patients and doses, patient tissue slices were imaged with LI-COR Pearl, the standardizable close field camera, using the standard postoperative workflow for fluorescence. In all patients with histopathologically proven viable tumor tissue, tumor tissue showed a higher fluorescence signal intensity with a sharp morphological delineation on tissue slices compared to normal tissue, irrespective of dose and tumor type. MFI increased slightly with dose in the dose range studied, however, CNR and TBR was variable and remained high and did not show any systematic variation with dose or tumor type. An ROC curve analysis performed at the measurement level on these tissue slices showed an area under the curve of 0.9726, P<0.0001, showing excellent performance. These data support a highly sensitive and specific and tumor agnostic performance characteristics of Compound 1.

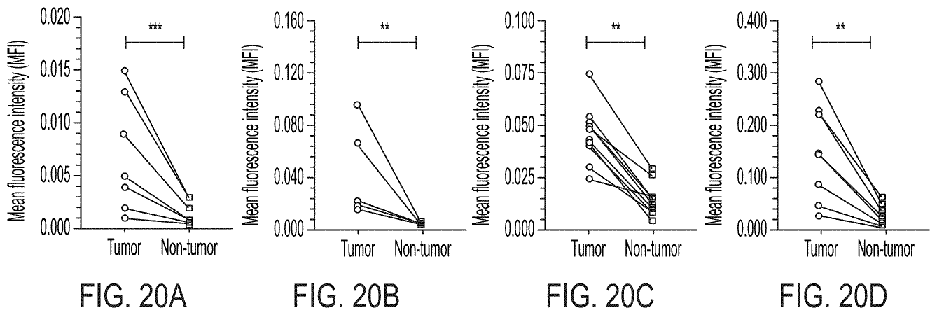

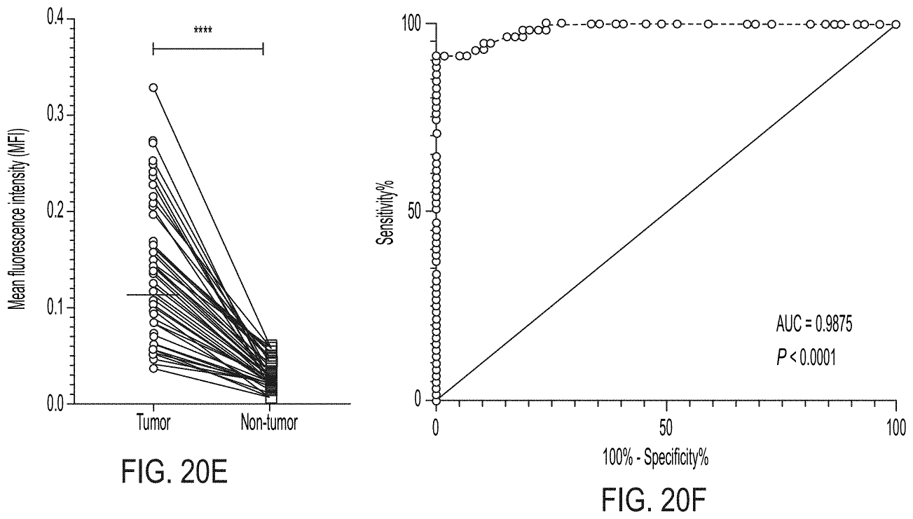

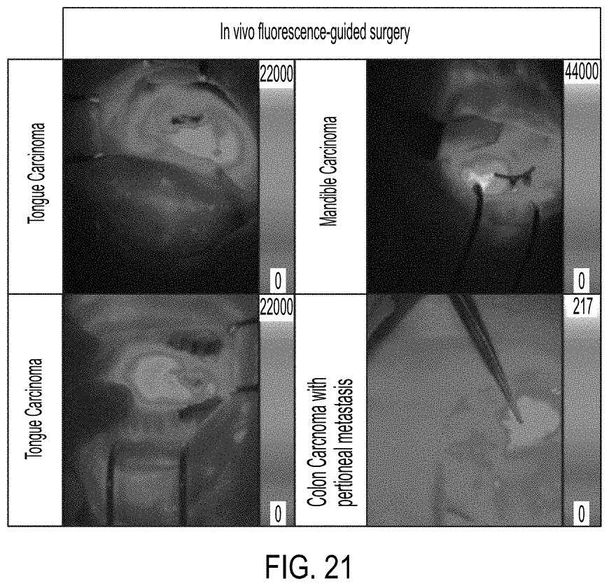

Ex vivo workflow analysis, to further validate the intraoperative finings, showed that the tumor tissue of all the subjects with histopathologically proven viable tumor tissue showed a higher fluorescence signal intensity with sharp morphological delineations in the tissue slices compared to normal tissue, irrespective of tumor type and dose cohort (FIG. 16, panel y). The tumor's mean fluorescence intensity (MFI) increased with dose (FIG. 20, panel a). In all the cohorts, the tumor MFIs were significantly higher than that of non-tumor tissue. The median tumor-to-background ratio (TBR) of all the tissue sliced (n=97 from 26 subjects) was 4.5 with an interquartile range (IQR) of 3.1. The optimal dose for tumor detection and sensitivity according to Phase 1b studies was 1.2 mg/kg (TBR 4.5, IQR 3.0) and MFI of the dose group's tumor tissue was significantly higher compared with normal tissue in each of the available tissue slices. A receiver-operator characteristic (ROC) curve analysis of these tissue slices showed an AUC of 0.9875 (FIG. 20, panel g).

### Example 14. Nanoscale Macromolecular Cooperativity Response to Tumor Acidosis for Image Guided Cancer Surgery

In this first-in-human fluorescence image-guided surgery study, compelling in vivo and ex vivo data indicates that low pH resulting from tumor acidosis can be exploited as a tumor agnostic biomarker for cancer in patients with a variety of solid tumors including HNSCC, BC, EC, and CRC. The pH-sensitive fluorescent imaging agent Compound 1 was specifically and durably activated by tumor acidosis, sharply delineating tumors from normal tissue and in several cases provided information on occult cancer not obtained by the SOC: intraoperative detection of all positive margins (9 out of 9), DCIS, and a satellite cancer in a patient with HNSCC, as well as ex vivo detection of 3 additional satellite lesions and second primaries in pathology specimens.

Successful clinical exploitation of tumor pH for imaging was possible due to the design of Compound 1, overcoming metabolic and phenotypic variability between different patients and tumors. It was feasible to detect all histological proven tumor positive surgical margins (9 out of 9) using Compound 1 fluorescence imaging. Most importantly, there was no overlap between tumor and background fluorescence for any given patient. The suppression of background activation and complete and irreversible unquenching at the threshold acidic pH due to the cooperative behavior of the pH responsive unimers has been described. This cooperativity, not predicted by studying individual unimers, is an emergent phenomenon from multiple separate polymers interacting as micelles and is responsible for the clinical effects we have observed.

### Conclusions

Accurate and unambiguous delineation of cancer location is required for clinical success, as surgeons typically already have extensive information on the locations of tumors. The ability of an optical imaging output to improve surgical outcomes is predicated on delivering information the surgeon does not have from pre-operative imaging and intraoperative inspection. The additional information from Compound 1 not provided by the SOC has the potential to significantly impact clinical care.

In this first-in-human Phase 1 study:
Compound 1 fluorescence imaging was feasible in all 4 tumor types evaluated (HNSCC, BC, CRC, or EC), demonstrating feasibility for tumor agnostic imaging with Compound 1 as expected by its mechanism of action.
Compound 1 fluorescence exhibited sharp demarcation between histology confirmed tumor versus normal tissue, with high CNR and TBR values, a critical factor for real-time image-guided surgery.
Compound 1 imaging detected all 9 tumor positive margin patients using in vivo wound bed imaging combined with the back-table imaging of the excised specimen within 1 hour of excision. In vivo wound-bed imaging detected 2 other occult tumors that were missed by routine surgery and were confirmed by standard pathology, demonstrating potential for significant value of Compound 1 image-guided surgery in clinical decision making and patient management.
Compound 1 fluorescence was detectable by multiple NIR cameras used in the study (NOVADAQ SPY Elite, SurgVision Explorer Air, and LI-COR Pearl Imaging system).

Therefore, Compound 1, an intravenously administered, pH-activatable, NIR fluorescent imaging agent, allows both in vivo and back-table fluorescence visualization with a clear delineation of solid tumors (HNSCC, BC, CRC, and EC) from normal tissue. The results demonstrate the ability of Compound 1 to detect, otherwise missed, all tumor positive surgical margins and occult disease in multiple patients and displays tumor agnostic fluorescent visualization of tumors in all investigated tumor types. These data highlight significant potential for Compound 1 in clinical decision making in treatment plans and patient management during and post-surgery.

### Example 15. Evaluation of Breast, HNSCC, Prostate, and Ovarian Tumors 3-6 Hours Post Dosing from Multiple NIR Camera Systems and Multiple Clinical Trial Sites from Initial Phase 2 Studies

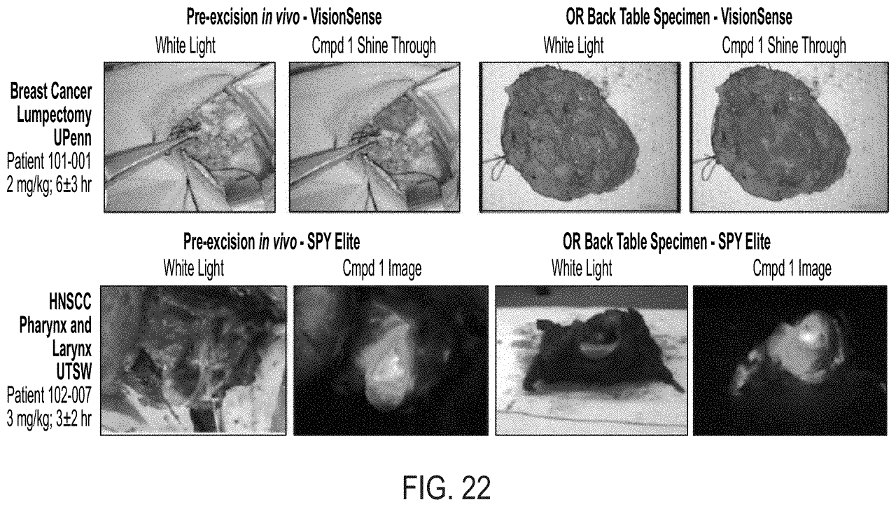

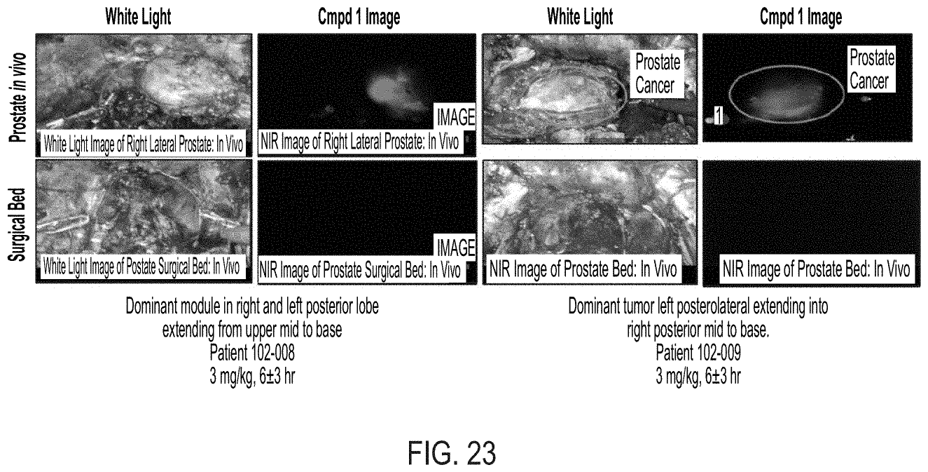

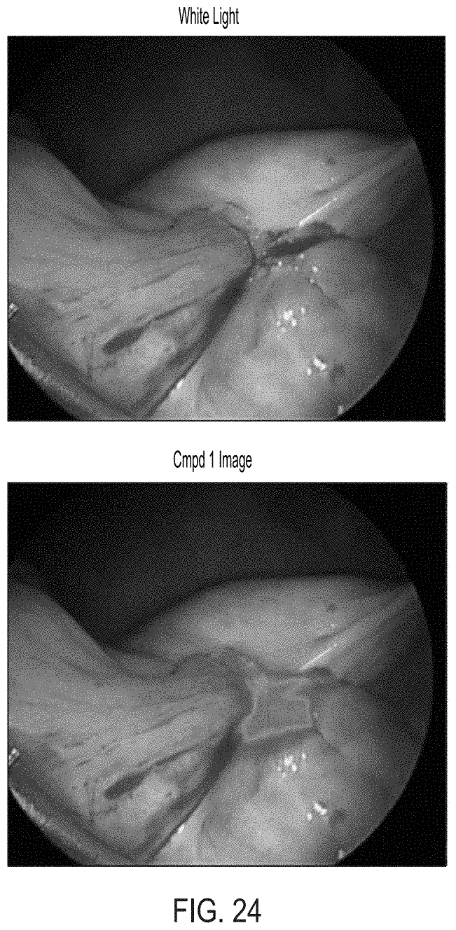

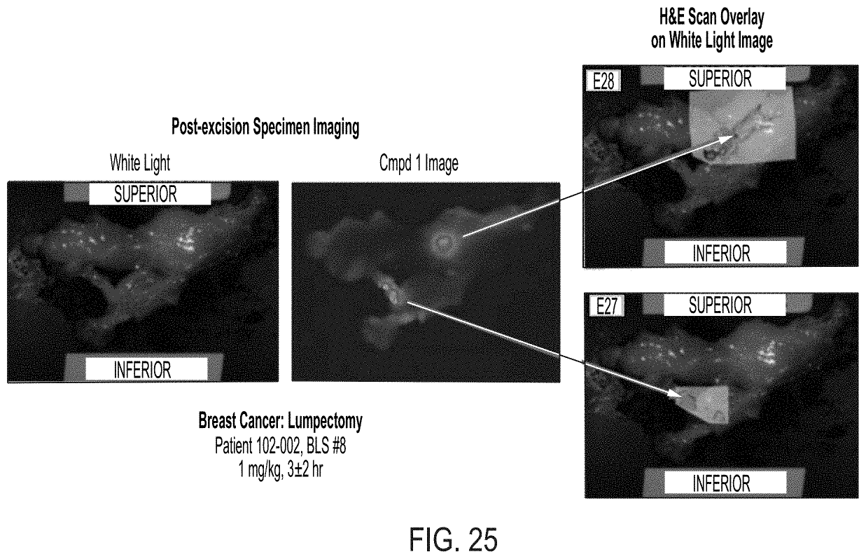

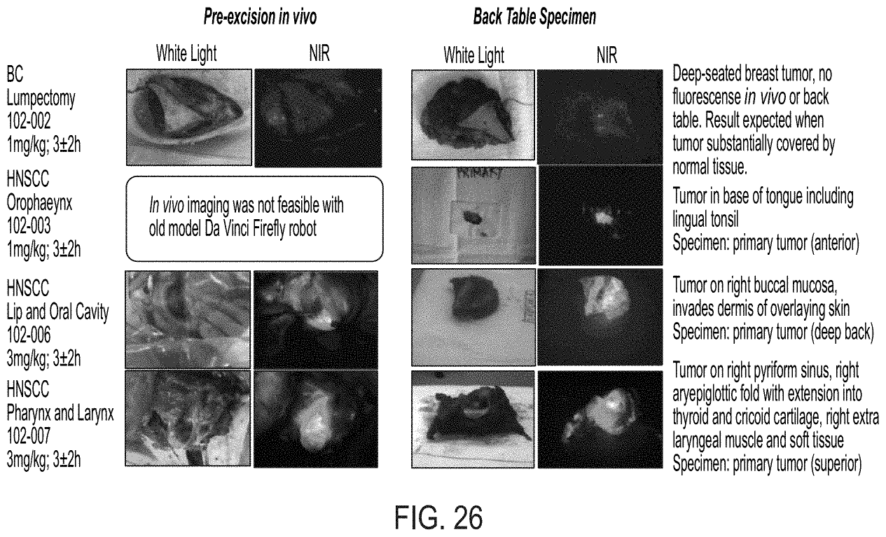

The ability to image tumors 3-6 hours after I.V. injection of Compound 1 was demonstrated for patients with breast cancer, HNSCC, prostate cancer, and ovarian cancer during a Phase 2 clinical study (FIGS. 22-26). The study also utilized data collected from different NIR cameras and from multiple sites. All patients received a single I.V. dose of Compound 1, followed by routine surgery approximately 3-6 hours after infusion of Compound 1. Pre-excision and post-excision intraoperative and backtable visualization a tumor from a breast cancer patient (101-001; UPenn; VisionSense NIR camera) dosed with Compound 1 (2 mg/kg) 6±3 hr prior to surgery and an HNSCC cancer patient (102-007; UTSW; NOVADAQ SPY Elite NIR camera) dosed with Compound 1 (3 mg/kg) 6±3 hr prior to surgery are shown in FIG. 22. In each case, white light imaging of the pre- or post-excised tumor/specimen is juxtaposed with an overlay of the fluorescence observed and the white light image, indicating the presence of the tumor. Intraoperative/in vivo imaging of prostate cancer from two patients (102-008 and 102-009; UTSW; Da Vinci Firefly NIR camera with updated software/hardware) dosed with Compound 1 (3 mg/kg) 6±3 hr prior to excision of the tumor and the imaging of wound bed after excision of the tumor are shown in FIG. 23. In each case, white light imaging of the pre-excised tumor/specimen and the surgical wound bed are juxtaposed with images of the fluorescence observed. The data show fluorescence from the tumor prior to resection and the absence of fluorescent in the surgical wound bed post-resection. A tumor from a patient (101-005) with ovarian cancer dosed with Compound 1 (3 mg/kg, 6±3 hr) was imaged in vivo pre-excision as shown in FIG. 24. A white light image juxtaposed with an overlay of the fluorescence observed and the white light image, indicating the presence of the tumor. The data from FIGS. 22-26 demonstrate the ability for Compound 1 to image tumors 3-6 hr post-dosing and using multiple types of NIR cameras as well as different clinical sites.

### Example 16. Evaluation of Tumor Selective Imaging Agent in Dogs with Solid Neoplasia

Materials and methods: After evaluation and recruitment for the study, dog-patients underwent (A) pre-operation analysis to identify possible types of lesion, and (B) Compound 1 tracer was administrated at 0.5 to 2.0 mg/kg, 18-78 hours prior to surgery. During the surgery (C) intra-operative imaging was performed using a Hamamatsu PDE or custom NIR camera before and after tumor removal (or after limb amputations). Resected tissues were (D) imaged with a LI-COR Pearl Imaging station and tumor-to-normal-tissue ratios were calculated accordingly. The resected tissues were then (E) preserved for histopathology validation. Safety was assessed separately in terms of adverse effects through physical examinations, laboratory tests and the recording of adverse events from infusion through discharge from hospital.

Results: A summary of the data from spayed or neutered dog patients that were recruited for the study is shown below (Table 16). Results from a total of seven dogs of different breeds, aged 4-12 years, body weights ranging from 20.9-59.5 kg, and with a range of tumors ware presented, including instances where more than one tumor was present. Doses studied thus far ranged from 0.5-2.0 mg/kg. In almost all instances, some pre-operative testing such as radiography, bone biopsies, or fine needle aspiration and cytology was performed, and this is captured in the footnotes in the table. As per the procedure described above, Compound 1 was administrated to the animals (“Dose”) and after 24 or 72 hr (“Time”) surgery commenced to remove the tumors. The resected tissues were sent to a veterinary pathologist for confirmation of the lesion which is noted along with the anatomical location in the table. Both acute and chronic adverse effects were monitored from the time of injection through discharge of the animals from the hospital and follow-up appointments (to remove sutures) and noted.

TABLE 16
Dog-patient information, Compound 1 dose regimen and histopathology study

| | | | | Wt. | Dose | Time | | Tumor Type by |
|---|---|---|---|---|---|---|---|---|
| ID | Age | Breed | Sex | (kg) | (mg/kg) | (hr) | Location | Histopathology |
| 1 | 8 | Labrador | SF | 30.2 | 0.5 | 24 | Left lateral | Soft tissue |
| | | Mix | | | | | maxillary lip | sarcoma |
| | | | | | | | caudoventral | |
| | | | | | | | to nose | |
| 2 | 4 | Great Dane | SF | 59.5 | 1 | 24 | Left distal tibia | Osteosarcoma |
| 3 | 10 | Pit Bull | SF | 29.1 | 1 | 24 | Left pinna of ear | Soft tissue |
| | | | | | | | | sarcoma |
| 4 | 5.5 | Brittany | SF | 20.9 | 1 | 24 | Left popliteal | Soft tissue |
| | | Spaniel | | | | | lymph node | sarcoma |
| 5 | 9 | Shar-Pei | SF | 23.0 | 2 | 24 | Left caudal hip | Fibro adnexal |
| | | Mix | | | | | | hamartoma |
| 6 | 12 | Golden | SF | 36.8 | 0.5 | 72 | Left lateral thigh | Mast cell tumor |
| | | Retriever | | | | | | |
| 7 | 6 | Mastiff | SF | 41.9 | 1 | 72 | Lateral thorax | Mast cell tumor |

SF = Spayed Female

The results from the study described in Table 16 demonstrated that (i) no adverse effects were observed for any of the dogs at any stage from injection of Compound 1 through their rehabilitation, post-surgery, (ii) fluorescent signals were observed where expected for diseased tissues based upon a combination of data from pre-operative biopsies and histopathology which was observed across a broad range of tumors, and (iii) in one instance, occult disease was identified during surgery for removal of a primary tumor.

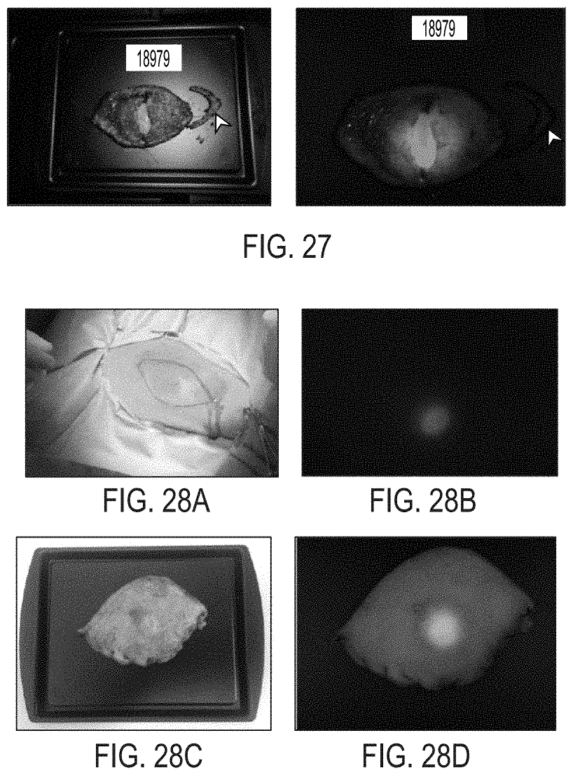

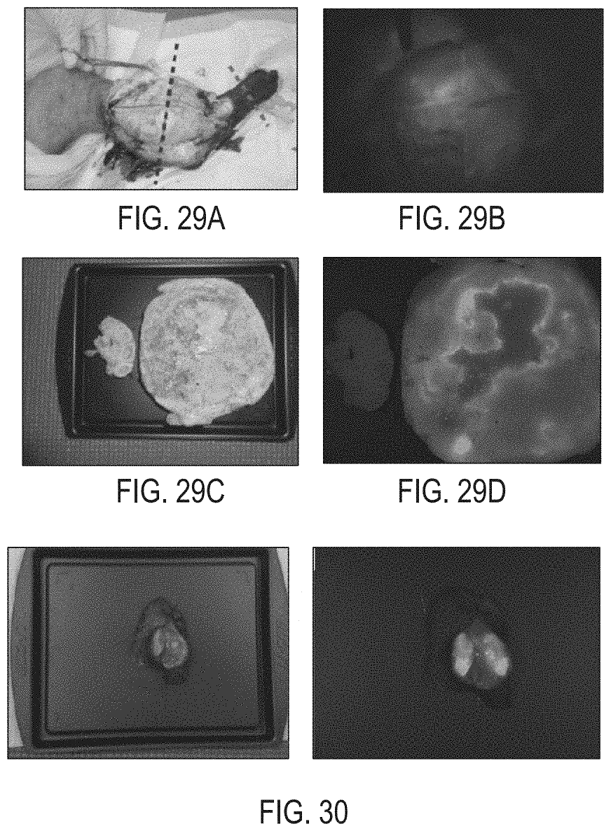

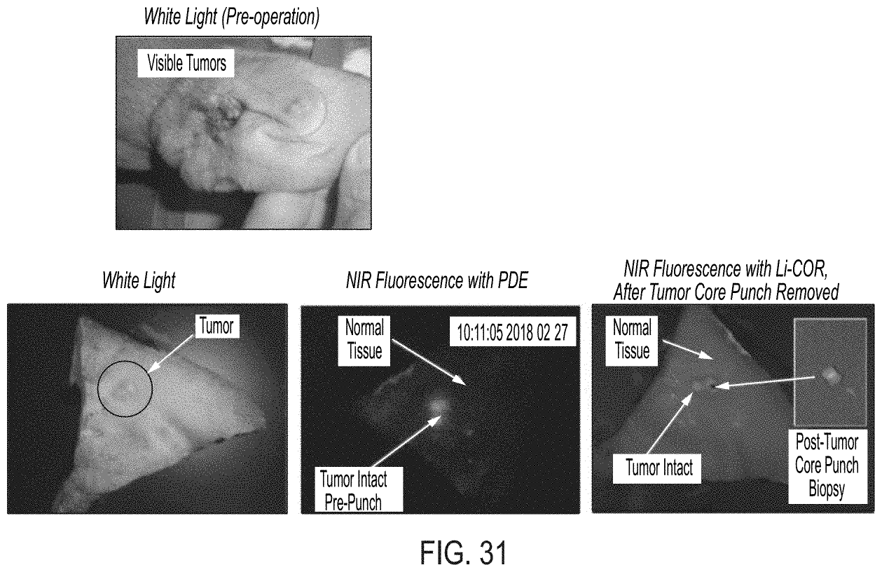

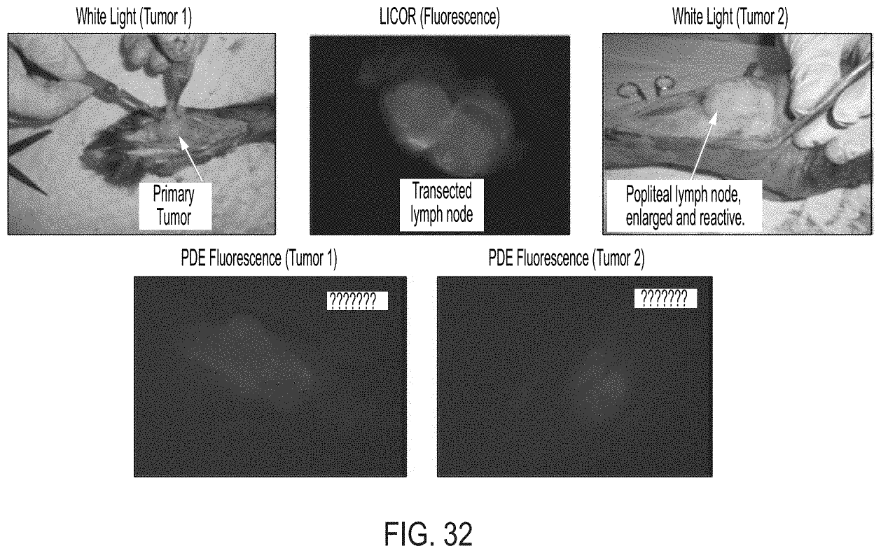

Results are shown for dog-patients in FIGS. 27-32 with white light as well as NIR fluorescent images using a LI-COR Pearl imaging station. FIG. 27 shows mast cell tumor resection. The white light image on the left side shows the resected tissue, and the tumor tissue also was revealed by performing a vertical excision. The suspected cancerous tissues are clearly evident in the NIR fluorescence image on the right side of the figure and are differentiated from a resected distal tissue (arrowhead) on the right side of each figure. FIG. 28 shows an osteosarcoma resected by limb amputation from dog-patient and imaged under white light. Ex vivo imaging was performed using a Hamamatsu PDE and a LI-COR Pearl. FIG. 32 shows a detection of occult disease in the distal soft tissue sarcoma in lymph nodes of a dog patient. During surgical removal of a primary soft tissue sarcoma located in the left metatarsal region from dog-patient, a lymph node was observed to be fluorescent and this was resected and imaged inter-operatively by white light and then in vivo using a Hamamatsu PDE NIR camera and ex vivo using the LI-COR Pearl NIR Imaging station.

Conclusion: A total of 7 dogs with osteosarcomas, soft tissue sarcomas, mast cell tumors, follicular cysts and other diseased tissues have been evaluated in a dog-patient study. The results obtained thus far have demonstrated: (i) no adverse effects for all dogs following injection of Compound 1 through hospital discharge, (ii) correlation of the Compound 1 derived location of cancerous tissues with data from physical examinations, from pre-operative biopsies, and post-excision histopathology for all malignant tumors tested; and (iii) identification of occult disease (a metastatic popliteal lymph node) in for one of the dog-patients in the study. Additionally, fluorescence imaging was possible with 3 cameras, all of which detect ICG, suggesting that imaging can be performed with any camera that is capable of detecting ICG fluorescence. These results support the safety of Compound 1 and its efficacy across a wide range of tumors that differ substantially in their oncogenic genotypes, and with dose regimens are clinically relevant to human trials.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A block copolymer having the structure of Formula (II), or a pharmaceutically acceptable salt, solvate, hydrate, or isotopic variant thereof: 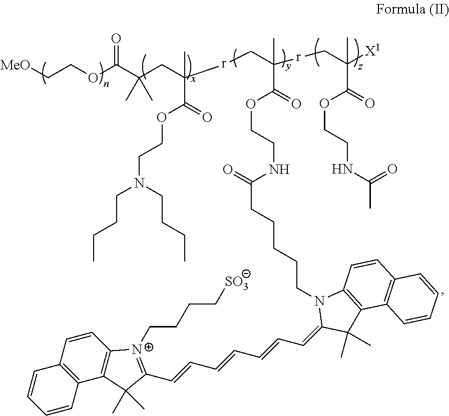
wherein:
X¹ is a halogen, —OH, or —C(O)OH;
n is 90-140;
x is 50-200;
y is 0-3; and
z is 1-3.

2. (canceled)

3. The block copolymer of claim 1, wherein X¹ is —Br.

4. The block copolymer of claim 1, wherein n is 100-120 and x is 60-150.

5-10. (canceled)

11. A composition or micelle comprising of one or more block copolymers of claim 1.

12. (canceled)

13. The pH responsive composition comprising the micelle of claim 11, wherein the micelle has a pH transition point and an emission spectrum, and the pH transition point is between 4.8-5.5 and the emission spectrum is between 700-900 nm.

14-15. (canceled)

16. The pH responsive composition of claim 13, wherein the composition has a pH transition range (ΔpH_{10-90%}) of less than 1 pH unit and the composition has a fluorescence activation ratio of greater than 25.

17-20. (canceled)

21. An imaging agent comprising one or more block copolymers of claim 1 wherein the imaging agent comprises poly(ethyleneoxide)-b-poly(dibutylaminoethyl methacrylate-r-aminoethylmethylacrylate hydrochloride) copolymer indocyanine green and acetic acid conjugate.

22. (canceled)

23. A pharmaceutical composition comprising a micelle, wherein the micelle comprises 1) one or more block copolymers having the structure of Formula (II), or a pharmaceutically acceptable salt, solvate, or hydrate: 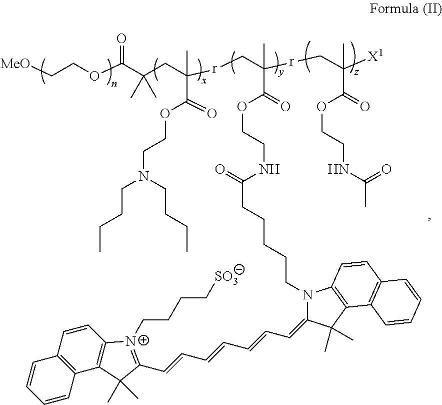
wherein:
X¹ is a halogen, —OH, or —C(O)OH;
n is 90-140;
x is 50-200;
y is 0-3;
z is 1-3; and
2) a stabilizing agent.

24. The pharmaceutical composition of claim 23, wherein the stabilizing agent is a cryoprotectant, a sugar, a sugar derivative, a detergent, or a salt.

25-28. (canceled)

29. The pharmaceutical composition of claim 23, wherein the stabilizing agent is trehalose.

30. The pharmaceutical composition of claim 23, comprising from about 0.5% to about 25% w/v of the stabilizing agent.

31. (canceled)

32. The pharmaceutical composition of claim 23, further comprising a liquid carrier, wherein the liquid carrier is sterile water, normal saline, half normal saline, 5% dextrose in water (D5W), ringers lactate solution, or a combination thereof.

33-39. (canceled)

40. A pharmaceutical composition, comprising 1) at least about 3 mg/mL of a block copolymer having the structure of Formula (II), or a pharmaceutically acceptable salt, solvate, or hydrate thereof: 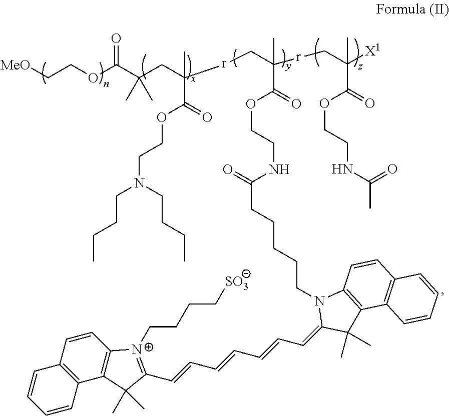
wherein:
X¹ is —Br;
n is 90-140;
x is 60-150;
y is 0-3;
z is 1-3; and
2) about 10% trehalose w/v in water.

41-42. (canceled)

43. A method of imaging the pH of an intracellular or extracellular environment, the method comprising:
a) contacting the intracellular or extracellular environment with a block copolymer of claim 1; and
b) detecting one or more optical signals from the intracellular or extracellular environment, wherein a detected optical signal indicates that the micelle comprising one or more block copolymers of Formula (II) has reached its pH transition point and disassociated.

44-54. (canceled)

55. The method of claim 43, comprising intravenously administering to the patient in need the pharmaceutical composition prior to a surgery.

56-57. (canceled)

58. A method of resecting a tumor in a patient in need thereof, the method comprising:
a) detecting one or more fluorescent optical signals from the tumor or a sample thereof from the patient administered with an effective dose of a block copolymer of claim 1, wherein a detected optical signal(s) indicates the presence of the tumor; and
b) resecting the tumor via a surgery.

59-65. (canceled)

66. The method of claim 58, wherein the cancer is breast cancer, head and neck squamous cell carcinoma (NHSCC), lung cancer, ovarian cancer, prostate cancer, bladder cancer, urethral cancer, esophageal cancer, brain cancer, pancreatic cancer, skin cancer, melanoma, sarcoma, pleural metastasis, kidney cancer, lymph node cancer, cervical cancer, or colorectal cancer.

67. (canceled)

68. The method of claim 58, wherein the pharmaceutical composition is administered as an injection or an infusion.

69. (canceled)

70. The method of claim 58, wherein the pharmaceutical composition is administered at least 1 hour prior to a surgery.

71. (canceled)

72. A method of treating cancer, the method comprising:
a) detecting one or more fluorescent optical signals in a cancer patient in need thereof administered with an effective dose of a block copolymer of claim 1, wherein a detected optical signal indicates the presence of a cancerous tumor; and
b) removing the cancerous tumor, thereby treating the cancer.

73-85. (canceled)
